(19) United States
(12) Patent Application Publication
(10) Pub. No.: US 2026/0297684 A1
(43) Pub. Date: Oct. 1, 2026

(54) METHOD FOR DETERMINING RESISTANCE TO IMMUNE CHECKPOINT INHIBITORS

(71) Applicant: New York University, New York, NY (US)

(72) Inventors: Tomas KIRCHHOFF, New York, NY (US); Robert FERGUSON, New York, NY (US); Leah Jo MORALES, New York, NY (US); Kelsey Renee MONSON, New York, NY (US); Jeffrey WEBER, New York, NY (US)

(21) Appl. No.: 19/490,171

(22) PCT Filed: Jun. 7, 2024
(86) PCT No.: PCT/US2024/032981

§ 371 Date: Dec. 4, 2025

Related U.S. Application Data
(60) Provisional application No. 63/506,673, filed on Jun. 7, 2023.
(60) Provisional application No. 63/508,734, filed on Jun. 16, 2023.

(51) Int. Cl. C12Q 1/6886; C07K 16/28

(52) U.S. Cl. CPC C12Q 1/6886; C07K 16/2818; C12Q 2600/106; C12Q 2600/156; C12Q 2600/172

(57) ABSTRACT

Provided are methods for determining responsiveness to immune checkpoint inhibition. The methods involve determining a mitochondrial haplogroup for an individual who has been diagnosed with cancer. Based on the determination of the mitochondrial haplogroup, an immune checkpoint inhibition therapy can be selected for the individual with improved likelihood of efficacy compared to selection of an immune checkpoint inhibition therapy selected without the mitochondrial haplogroup analysis. The mitochondrial haplogroup analysis can be performed prior to initiating an immune checkpoint therapy.

### CROSS REFERENCE TO RELATED APPLICATIONS

This application is based on and claims the benefit of U.S. Provisional Patent Application No. 63/506,673, filed on Jun. 7, 2023, and of U.S. Provisional Patent Application No. 63/508,734, filed on Jun. 16, 2023, the entire disclosures of which are incorporated herein by reference.

### STATEMENT REGARDING FEDERALLY SPONSORED RESEARCH

This invention was made with government support under R01 CA227505 and F99 CA274650 awarded by the National Institutes of Health. The government has certain rights in the invention.

### RELATED INFORMATION

While the prognosis for early-stage melanoma is favorable, with a 10-year survival rate of >99%, late-stage disease was long considered incurable. Immune checkpoint inhibitors (ICIs) have revolutionized melanoma treatment of advanced disease. Single-agent anti-PD1 or the combination of anti-CTLA4 and anti-PDl^{1,2} are now the primary frontline therapies for advanced melanoma, often producing curative outcomes. Nevertheless, ~40% of patients do not respond across treatment arms^{3,4} and ~80% of ICI-treated patients experience immune-related adverse events (irAEs), frequently resulting in treatment discontinuation that dramatically reduces therapy benefits.⁵⁻⁷ The availability of reliable biomarkers predictive of ICI efficacy and/or toxicity at the baseline (pre-treatment) would have significant clinical benefit for improving current ICI regimens and patient survival.

Existing biomarkers of ICI efficacy (response, survival), such as tumor mutation burden (TMB)⁸⁻¹⁰ or levels of infiltrating lymphocytes (TILs),¹¹ are primarily derived from the tumor and tumor microenvironment (TME) and have demonstrated clinical utility in predicting ICI efficacy.^{12,13} Nevertheless, these alone or in combination do not sufficiently explain the heterogeneity of the outcomes observed in ICI-treated patients. Furthermore, the current tumor-based predictors introduce several challenges for clinical practice. These include concerns regarding the feasibility of routine tumor-based assays, and the biological constraints of tumor heterogeneity, in particular for metastatic lesions eliciting diverse immune responses in distal metastatic sites or the shifting tumor/immunity dynamics during ICI treatments. A recent study also showed variation in TMB between different lesions collected posthumously from the same patient,¹⁴ highlighting previous challenges inherent in reliable and reproducible tumor/immune repertoire assessment.

Over the last several years mitochondrial function has become increasingly implicated in T-cell proliferation and memory formation.^{15,16} Mitochondria are providers of energy in the cell via adenosine triphosphate (ATP) production and mitochondrial assembly requires proteins that arise from both the nuclear and mitochondrial genomes. Unlike the nuclear genome, the mitochondrial genome is inherited solely from the maternal parent and all mitochondrial diversity can be categorized into haplogroups which are defined by specific sets of single nucleotide polymorphisms (SNPs). Haplogroups are associated with different ethnic backgrounds representing major branch points in the mitochondrial phylogenetic tree as humans dispersed globally from Africa. Experiments using cybrids (cell lines with identical nuclear genomes but different mitochondrial genomes) have shown that different mitochondrial haplogroups are associated with changes in a cell's ability to resist chemotherapy drugs¹⁷ and reactive oxygen species (ROS).^{18,19}

Mitochondrial function has also been linked to ICI outcomes with modulation of T-cell efficacy being one proposed mechanism. Recent work has identified a subset of CD8+ cells present in both peripheral blood and tumor-infiltrating T-cells with high levels of oxidative phosphorylation (OXPHOS) as well as both cytotoxicity and exhaustion markers (CD8+ TOXPHOS cells). High levels of CD8+ TOXPHOS cells in the ICI-treated patient cohort were correlated with resistance to treatment.²⁰ Furthermore, they developed a predictive model based on the single-cell transcriptomic profile of CD8+ TOXPHOS cells which was able to predict patient outcomes with ~88% accuracy in their scRNAseq and validation cohorts.²⁰

In mouse models, reactive oxygen species (ROS) signaling has also been demonstrated to improve the tumor-killing effect seen when disrupting the PD-1/PD-L1 axis.²¹ Injection of a ROS precursor (Luperox) into mice with MC38-derived tumors did not increase antitumor activity alone. When administered in combination with anti-PD-L1 however, the antitumor effect was greater than what was observed with single-line anti-PD-L1 treatment, and this improved antitumor effect was lost when ROS was quenched.²¹ They further demonstrated that activation of AMPK and mTOR pathways, which are both downstream of ROS signaling, also synergized with anti-PD-L1 to increase antitumor activity with the activation of both simultaneously providing the best response.²¹ The AMPK and mTOR signaling pathways increase the expression of PGC-1α which activates mitochondria through nuclear respiratory factors (NRFs) and peroxisome proliferator-activated receptors (PPARs). Two drugs known to activate PGC-1a/NRF2 (oltipraz) and PGC-1a/PPARs (bezafibrate) were also tested for synergistic effects with anti-PD-L1 in mouse models and both showed improved tumor control when tested in combination with anti-PD-L1 but not when used as single-line therapies. Further studies demonstrated that activation of mitochondria using bezafibrate can overcome the immunosuppressive factors secreted by some cancers that inhibit tumor control by anti-PD-L1,²² increasing both proliferation of naïve T-cells and the numbers of effector/memory T-cells.²³ While these studies provide evidence that mitochondria are an important regulator of ICI response, there remains an ongoing and unmet need for improved approaches to predicting ICI responses, and for adjusting patient treatment based on such predictions. The present disclosure is pertinent to this need.

### BRIEF SUMMARY

ICIs have revolutionized metastatic melanoma treatment; however, a significant proportion of patients do not respond to treatment. The present disclosure describes minimally invasive pre-treatment biomarkers to stratify melanoma patients so that they can be treated with the correct treatment regimen. The present disclosure demonstrates that baseline host immunity and mitochondrial haplogroups represent novel biomarkers of ICI resistance. Using samples and clinical data from both clinical trials and a large standard-of-care (SOC) cohort, we assessed the association of peripheral CD8+ T cell expression and underlying genomic and mitochondrial genetic repertoire with outcomes in multiple ICI treatment regimens. Combining RNA-seq, ATAC-seq, and High-coverage (30x) Whole Genome Sequencing (WGS) of baseline samples from 115 anti-PD-1 (nivolumab “NIVO”)-treated metastatic melanoma (MM) patients from the CheckMate 067 BMS Phase III clinical trial we determined transcriptomic and genetic underpinnings, comparing patients with NIVO clinical benefit (CB) and no clinical benefit (NCB). We additionally assessed differences in pre-treatment single-cell transcription profiles of cytotoxic T-cells of these patients by performing single-cell (sc) RNA-seq on CD8+ T-cells from baseline PBMCs. We then validated these findings in additional NIVO-treated MM patients from the CheckMate 067 trial using 30× WGS, ultra-low-coverage (0.5×) (1c) WGS, and Whole Exome Sequencing (WES). We further validated these findings in in NIVO-treated MM patients in the IO-GEM cohort, an international consortium of genomic data collected in the SOC setting. Mitochondrial haplogroups in IO-GEM were determined from both Global Screening Array (GSA) and 0.5× lcWGS. Additional validation of the findings was performed in anti-CTLA-4 ipilimumab (IPI)-treated CheckMate 067 and IO-GEM patients. We assessed the association of transcriptomes and haplogroups with combination therapies, first in IPI/NIVO-treated CheckMate-067 patients, and then IPI/NIVO IO-GEM patients. Based on the described analysis, the disclosure provides novel biomarkers detected by RNA-seq analyses of CD8+ T cells (HMAX) pointing to MTRNR2L8 as the most significantly associated with NIVO resistance, in addition to other informative markers as further described herein. Combined with genomic analyses the disclosure reveals that this association is driven by the genetic variation that determines specific mitochondrial haplogroup repertoire. The disclosure also provides an analysis showing that HMAX-patients are enriched by most common European haplogroup (H, ~45%), displaying a NIVO response distribution (~48% CB, 52% NCB). The disclosure demonstrates that HMAX association detected in CD8+ RNA-seq analysis (in HMAX+ patients) links with T haplogroup, as the most significant haplogroup that is ICI resistant. Response rates differ significantly when assessed across the other major European haplogroups. CheckMate-067 patients with the T haplogroup fare especially poorly when treated with NIVO, with a 79% NCB rate (NCB OR, T vs. Others=3.99 (1.65-11.2), p-value=2.26E-03), a finding that has been validated in IO-GEM (NCB OR, T vs. Others=3.00 (1.10-9.59), p-value=0.04. From scRNA-seq, the disclosure provides a novel CD8+ T cell-type-specific phenotype among T haplogroup patients, composed of predominantly naïve or undifferentiated T-cells. In contrast, T haplogroup patients treated with IPI tended to have better outcomes compared to the others. Outcomes for T haplogroup patients treated with combined IPI/NIVO are also significantly worse when compared to other haplogroups (69% NCB for HG-T compared to 37% NCB in other haplogroups).

Thus, the disclosure demonstrates for the first time that response to ICI differs significantly across mitochondrial haplogroups, and different ICI treatments produce different outcomes within mitochondrial haplogroups. The disclosure also reveals a unique pre-treatment cellular phenotype among cytotoxic T-cells in T haplogroup patients showing a greater proportion of naïve cells. Given the provided evidence that metabolic differences across haplogroups affect T-cell differentiation, the disclosure provides identification of a subset of patients for whom ICIs targeting cellular differentiation (e.g., anti-CTLA-4) will likely produce better treatment outcomes. However, the disclosure also provides identification of patients that would reach substantially better clinical benefit from ICI that reinvigorate exhausted cells (anti-PD-1) if treated by pembrolizumab. The disclosure therefore provides improved pre-treatment identification of patients across ICI therapies in melanoma and potentially other cancers, thereby providing for tailoring existing ICI treatments to the appropriate patients and personalized combination ICI therapies. Identifying immune checkpoint inhibition responses as associated with other haplotypes are also provided, such as for K, J and U haplogroups. Markers for L haplogroups and others are also provided and can be analyzed for use in predicting immune checkpoint inhibition responses. Accordingly, the disclosure identifies variants associated with a variety of mitochondrial haplogroups as an independent method of determining ICI outcomes at baseline. In examples, the disclosure accordingly provides a method comprising determining whether or not an individual is likely to respond to treatment with one or a combination of particular ICI's based on a determination of a mitochondrial haplogroup for the individual, and wherein the haplogroup of the individual is determined prior to receiving immune checkpoint inhibition. In examples, the haplogroup is a T haplogroup, a K haplogroup, a U haplogroup, or a J haplogroup. In an example, the haplogroup is the T haplogroup or the K haplogroup or the U haplogroup, and wherein the T haplogroup or the K haplogroup or the U haplogroup indicates the individual is likely to be resistant to an anti-PD-1 ICI as a monotherapy. In this example, a method of the disclosure may further comprise administering to the individual an ICI that is not anti-PD-1 therapy as a monotherapy. In an example, the haplogroup is the J haplogroup, thereby indicating the individual will likely be sensitive to an anti-PD1 ICI. In this example, the method may further comprise administering the anti-PD-1 ICI to the individual. Data presented in this disclosure demonstrate that individuals with haplogroup T are more sensitive to pembrolizumab and resistant to nivolumab.

In examples, determining the haplogroup comprises determining one or more of the markers as defined by the haplogroup tables herein, including but not necessarily limited to Tables 1-4, and the Haplogroup Tables. In an example, the disclosure provides for quantitative next-generation sequencing analysis of RNA expressed from a locus that includes MTRNR2L8 or ATAC-seq chromatin accessibility at this locus (e.g., a quantitative DNA-based method), to thereby indicate the individual has the T haplogroup. In this example, the method may further comprise administering to the individual an ICI that is not anti-PD-1 therapy as a monotherapy.

### BRIEF DESCRIPTION OF THE FIGURES

FIG. 1. Population flow diagram illustrating the full patient population, sample collection, multi-omic material extraction, sequencing/genotyping, and haplogroup determination for the analytic cohorts.

FIG. 2A. Violin plot of the meta-analysis within the single-agent Discovery and Validation Cohorts showing the distribution of HMAX log 2 (gene expression) in baseline (pre-treatment) peripheral blood CD8+ T-cells of patients treated by single-agent Nivolumab ICI by response status (n=116; Complete Response (CR), n=21; Partial Response (PR), n=27; Stable Disease (SD), n=9; Progressive Disease (PD), n=59). HMAX was identified by RNA-seq differential expression as the most significantly differentially expressed gene in this cohort (FDR adj. p=2.79E-13, log 2FC=4.03).

FIG. 2B. Violin plot of the meta-analysis within the combination Discovery and Validation Cohorts showing the distribution of HMAX log 2 (gene expression) in baseline (pre-treatment) peripheral blood CD8+ T-cells of patients treated by combination Ipilimumab/Nivolumab ICI by response status (n=115; Complete Response (CR), n=26; Partial Response (PR), n=42; Stable Disease (SD), n=15; Progressive Disease (PD), n=32). HMAX was identified by RNA-seq differential expression as the most significantly differentially expressed gene in this cohort (FDR adj. p=3.12E-28, log 2FC=4.95).

FIG. 3A. Volcano plot of the meta-analysis within the single-agent Discovery and Validation Cohorts showing regions of differentially accessible chromatin identified by ATAC-seq analysis in baseline (pre-treatment) peripheral blood CD8+ T-cells of patients treated by single-agent Nivolumab ICI by clinical benefit (CB) status (n=116; CB (Complete Response (CR), n=21; Partial Response (PR), n=27) vs. No Clinical Benefit (NCB) (Stable Disease (SD), n=9; Progressive Disease (PD), n=59); Purple points are regions of open chromatin with a p-value for significant differential accessibility >0.005; gray points are regions with an abs (log 2FC)<1; pink points are regions with a p-value for significant differential accessibility≤0.05 and an abs (log 2FC) ≥1; the green highlighted point is the region of significantly differentially accessible chromatin near the HMAX locus (chr11: 10,507,914-10,509,996, build hg38) (FDR adj. p=7.8686E-08, log 2FC=−1.48).

FIG. 3B. Violin plot of the meta-analysis within the single-agent Discovery and Validation Cohorts showing the distribution of log 2 (chromatin accessibility) at the HMAX locus (chr11: 10,508,542-10,509,994, build hg38) from the ATAC-seq analysis in baseline (pre-treatment) peripheral blood CD8+ T-cells of patients treated by single-agent Nivolumab ICI by response status (n=116; Complete Response (CR), n=21; Partial Response (PR), n=27; Stable Disease (SD), n=9; Progressive Disease (PD), n=59). The HMAX chromatin locus was identified by ATAC-seq differential chromatin accessibility as the most significantly differentially accessible region in this cohort (FDR adj. p=7.86E-08, log 2FC=−1.48).

FIG. 4A. Volcano plot of the combination Meta-Analysis Cohort showing the regions of differentially accessible chromatin identified by ATAC-seq analysis in baseline (pre-treatment) peripheral blood CD8+ T-cells of patients treated by combination Ipilimumab/Nivolumab ICI by Clinical Benefit (CB) status (n=110; CB (n=65, Complete Response (CR), n=23; Partial Response (PR), n=42) vs. No Clinical Benefit (NCB) (n=45, Stable Disease (SD), n=16; Progressive Disease (PD), n=29). Purple points are regions of open chromatin with a p-value for significant differential accessibility >0.005; gray points are regions with an abs (log 2FC)<1 and a p-value >0.005;); pink points are regions with a p-value for significant differential accessibility≤0.005 and an abs (log 2FC) ≥1; the green highlighted point is the region of significantly differentially accessible chromatin near the HMAX locus (chr11: 10,508,160-10,509,996, build hg38) (FDR adj. p=1.17E-03, log 2FC=−1.28).

FIG. 4B. Violin plot of the combination Meta-Analysis Cohort showing the distribution of log₂ (chromatin accessibility) at the HMAX locus (chr11: 10,508,160-10,509,996, build hg38) from the ATAC-seq analysis in baseline (pre-treatment) peripheral blood CD8+ T-cells of patients treated by combination Ipilimumab/Nivolumab ICI by response status (n=110; Complete Response (CR), n=23; Partial Response (PR), n=42; Stable Disease (SD), n=16; Progressive Disease (PD), n=29). The HMAX chromatin locus was identified by ATAC-seq differential accessibility among the top 10 most significantly differentially accessible regions in this cohort (FDR adj. p=1.17E-03, log 2FC=−1.28).

FIG. 5. Phylogenetic tree depicting mtDNA lineages and CheckMate-067 NIVO treatment response. Subjects with NIVO Clinical Benefit (CB; Complete Response (CR) or Partial Response (PR)) are shown in navy. Subjects with NIVO No Clinical Benefit (NCB; Stable Disease (SD) or Progressive Disease (PD)) are shown in blue. Frequency counts of CB and NCB are plotted for the major European haplogroups (H, U, T, J, K, V, I, X, W). Inset: proportion of CB and NCB within each major haplogroup (H, U, T, J, K).

FIGS. 6A-D. CheckMate-067 and IO-GEM NIVO treatment efficacy frequencies by European haplogroup (H, U, T, J, K, and Other, including W, R, V, X, I). (A) CheckMate-067 NIVO discovery cohort, (B) CheckMate-067 NIVO validation cohort, (C) CheckMate-067 pooled analysis, and (D) IO-GEM validation cohort.

FIG. 7. Forest plots showing the odds ratios (ORs), log odds, and p-values for NIVO treatment outcomes among T, T and K, or J haplogroup and Other European Haplogroups. Black boxes represent the log odds point estimate and the lines extending from them represent the 95% confidence intervals. Point estimates to the left of the dotted line indicate increased odds of NIVO clinical benefit, while point estimates to the right indicate increased odds of no NIVO clinical benefit. Confidence intervals that do not cross the dotted line indicate a statistically significant point estimate (p<0.05).

FIG. 8. Forest plot showing the odds ratios (ORs), log odds, and p-values when comparing NIVO treatment outcomes by haplogroups, comparing T versus Other European Haplogroups. Black boxes represent the point estimate for the log odds and the lines extending from them represent the 95% confidence intervals. Point estimates to the left of the dotted line indicate increased odds of NIVO clinical benefit, while point estimates to the right indicate increased odds of no NIVO clinical benefit. Confidence intervals that do not cross the dotted line indicate a statistically significant point estimate (p<0.05).

FIGS. 9A-D. Single-cell (sc) RNA-seq analysis for pre-treatment circulating CD8+ T cells from CheckMate-067 NIVO patients (n=21. FIG. 9A displays Uniform Manifold Approximation and Projection (UMAP) dimensionality reduction plot. FIG. 9B displays a bubble plot showing per-cluster cell-type classification based on T cell marker gene expression. FIG. 9C displays a box plot showing single-cell CD8+ T-cell type proportions stratified by NIVO treatment response (Clinical Benefit, CB; No Clinical Benefit, NCB) and haplogroup (NIVO-resistant haplogroups, HGR; other haplogroups, HGO). FIG. 9D displays a scatterplot showing per-cluster mean expression of T cell exhaustion markers grouped by cell type.

FIG. 10A. Top 8 significantly differentially expressed genes per cluster from single-cell (sc) RNA-seq of peripheral blood CD8+ T cells isolated from n=21 NIVO-treated patients from CheckMate-067.

FIG. 10B. Per-cluster cell-type classification based on differential expression of marker genes from scRNA-seq of peripheral blood CD8+ T cells isolated from n=21 NIVO-treated patients from CheckMate-067.

FIG. 10C. Per-cluster gene expression of T cell exhaustion markers from scRNA-seq of peripheral blood CD8+ T cells isolated from n=21 NIVO-treated patients from CheckMate-067.

FIGS. 11A-F. Proportion of CB and NCB ICI treatment responses in NIVO, COMBO (IPI/NIVO), and IPI cohorts comparing HG-T and other (H, U, J, K, W, R, V, X, and I) MT-HGs in: (A) CM-067 NIVO cohort; (B) CM-067 COMBO cohort; (C) CM-067 IPI cohort; (D) IO-GEM NIVO cohort; (E) IO-GEM COMBO cohort (F) IO-GEM IPI cohort. Discovery (CheckMate-067, n=526) and Validation (IO-GEM, n=749). CB: clinical benefit (Complete Response CR; Partial Response PR; durable Stable Disease dSD). NCB: no clinical benefit (progressive Stable Disease pSD; Progressive Disease PD).

FIGS. 12A-B. ICI treatment efficacy in NIVO, IPI, or IPI/NIVO cohorts comparing HG-T and other (H, U, J, K, W, R, V, X, and I) MT-HGs. CB: clinical benefit (Complete Response CR; Partial Response PR; durable Stable Disease dSD). NCB: no clinical benefit (progressive Stable Disease pSD; Progression of Disease POD). MT-HG: mitochondrial haplogroups. FIG. 12A displays a meta-analysis proportions of CB and NCB comparing HG-T and other MT-HGs for: CheckMate-067 and IO-GEM IPI cohort (CTLA-4); CheckMate-067 and IO-GEM NIVO cohort (PD-1); CheckMate-067 and IO-GEM COMBO cohort (Combo). FIG. 12B displays forest plots showing the odds ratios (ORs), log odds, and p-values for ICI treatment outcomes among T haplogroup and Other European Haplogroups. Black boxes represent the log odds point estimate and the lines extending from them represent the 95% confidence intervals. Point estimates to the left of the dotted line indicate increased odds of ICI clinical benefit compared to the reference (Other European Haplogroup patients treated with anti-CTLA-4, anti-PD-1, or Combo), while point estimates to the right indicate increased odds of no ICI clinical benefit compared to the reference. Confidence intervals that do not cross the dotted line indicate a statistically significant point estimate (p<0.05).

FIG. 13A. ICI treatment efficacy in anti-CTLA-4 (IPI), anti-PD-1 (NIVO and PEMBRO combined), or combined IPI/NIVO (COMBO) cohorts. CB: clinical benefit (Complete Response CR; Partial Response PR; durable Stable Disease dSD). NCB: no clinical benefit (progressive Stable Disease PSD; Progression of Disease POD). MT-HG: mitochondrial haplogroups.

FIG. 13B. ICI treatment efficacy in anti-CTLA-4 (IPI), anti-PD-1 (NIVO and PEMBRO separated), or combined IPI/NIVO (COMBO) cohorts. CB: clinical benefit (Complete Response CR; Partial Response PR; durable Stable Disease dSD). NCB: no clinical benefit (progressive Stable Disease pSD; Progression of Disease POD). MT-HG: mitochondrial haplogroups.

FIG. 13C. ICI treatment efficacy in anti-CTLA-4 (IPI), anti-PD-1 (NIVO and PEMBRO combined), or combined IPI/NIVO (COMBO) cohorts comparing HG-T and other (H, U, J, K, W, R, V, X, and I) MT-HGs. CB: clinical benefit (Complete Response CR; Partial Response PR; durable Stable Disease dSD). NCB: no clinical benefit (progressive Stable Disease pSD; Progression of Disease POD). MT-HG: mitochondrial haplogroups.

FIG. 13D. ICI treatment efficacy in anti-CTLA-4 (IPI), anti-PD-1 (NIVO and PEMBRO separated), or combined IPI/NIVO (COMBO) cohorts comparing HG-T and other (H, U, J, K, W, R, V, X, and I) MT-HGs. CB: clinical benefit (Complete Response CR; Partial Response PR; durable Stable Disease dSD). NCB: no clinical benefit (progressive Stable Disease pSD; Progression of Disease POD). MT-HG: mitochondrial haplogroups.

FIG. 14. Volcano plot showing significant upregulation of ROS detoxification genes in baseline CD8+ T cells of HG-T patients from CheckMate-067 compared to HG-H (n=121)

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All nucleotide sequences described herein include the RNA and DNA equivalents of such sequences, i.e., an RNA sequence includes its cDNA. All nucleotide sequences include their complementary sequences. All protein sequences described herein include all isoforms of such proteins, e.g., proteins made from splice variants, and proteins that may vary from individual to individual in certain amino acids. All mRNAs described herein include all splice variants. The amino acid or polynucleotide sequence as the case may be associated any databased entry of this disclosure is incorporated herein by reference as presented in the database on the effective filing date of this application or patent.

In general, and as illustrated by the examples below and the figures, the present disclosure provides a method for predicting treatment response to immune checkpoint inhibitors (ICIs). The methods involve determining a mitochondrial haplogroup for an individual who has been diagnosed with cancer. Based on the determination of the mitochondrial haplogroup, an immune checkpoint inhibition therapy can be selected for the individual with improved likelihood of efficacy compared to selection of an immune checkpoint inhibition therapy selected without the mitochondrial haplogroup analysis. The mitochondrial haplogroup analysis can be performed prior to initiating an immune checkpoint therapy. Accordingly, the described methods are applicable to any individuals who are diagnosed with cancer. In examples, the method comprises a) analyzing a biological sample obtained from the individual prior to a treatment with one or more ICIs for expression of one or more biomarkers described herein, including but not necessarily limited to any one or combination of single nucleotide polymorphisms (SNPs) or other nucleotide changes, such as insertions or deletions, to establish a baseline level of the one or more markers, and b) comparing the signature of the one or more biomarkers to a control value, and/or designating the individual to have be of a particular mitochondrial haplogroup. While the disclosure relates in some examples to haplogroups that are identified by polynucleotide sequencing, the disclosure also includes analysis of proteins that may have certain features that indicate the individual is a member of a particular haplogroup. Thus, both nucleic acid and protein detection are included in the method. In examples, RNA sequencing is used. In examples, any suitable RNA-Seq approach may be used. In examples, mitochondrial DNA or RNA may be sequenced to determine a haplogroup. In examples, whole genome or whole exome sequencing may be used. In examples, quantitative next-generation sequencing analysis of RNA expressed from a locus that includes MTRNR2L8, or ATAC-seq chromatin accessibility at this locus, indicates the individual has the T haplogroup.

In examples, a biological sample from an individual is analyzed. In examples, a liquid biological sample is used. In examples, peripheral blood lymphocytes are obtained from an individual and are tested to determine a haplogroup. In examples, in addition to determining a haplogroup, the disclosure also comprises analyzing CD8+ T cells, and includes but is not necessarily limited to determining biomarker expression and/or a haplogroup in the CD8+ T cells. In examples, the control to which the patient sample is compared comprises a sample from one or more individuals who respond to one or more checkpoint inhibitors as a treatment for cancer, and as such, a control may be a value for a known haplogroup. In examples, the control comprises a sample from one or more individuals who respond to a combination of checkpoint inhibitors as a treatment for cancer. In examples, the control may be a standardized curve(s), a cutoff or threshold value, and the like. Where described haplogroup is determined, the disclosure further comprises administering one or more ICIs to the individual. In examples, a second, third, fourth, sample, etc. can be obtained from an individual who is undergoing treatment and tested to monitor the effect of the treatment, and keep steady, change, adjust, discontinue, or not reinitiate treatment with a checkpoint inhibitor.

In one example, the sample is obtained from an individual who has any type of cancer and has not undergone any previous ICI treatment, and thus may be considered a baseline sample. In one example, the individual has not received any ICI treatment within a period of at least six months. In one example, the cancer is melanoma, which may be metastatic melanoma, but the described methods can be used for other types of cancer, representative and non-limiting examples of which include lung cancer, breast cancer, ovarian cancer, pancreatic cancer, colorectal cancer, bladder cancer, brain cancer, cervical cancer, esophageal cancer, stomach cancer, head and neck cancer, kidney cancer, liver cancer, testicular cancer, prostate cancer, non-melanoma skin cancer, any sarcoma, uterine cancer, and liquid cancers of the blood or lymph, and any other type of cancer where use of any ICI treatment would be indicated.

The disclosure relates to predicting responses and recommending treatments, and providing treatments, for any of ICI that targets PD-1, non-limiting examples of which include Pembrolizumab, Nivolumab, and Cemiplimab, and any ICI that targets PD-L1, non-limiting examples of which include Atezolizumab, Avelumab, and Durvalumab, and any ICI that targets CTLA-4, non-limiting examples of which include Ipilimumab and Tremelimumab, and LAG-3 inhibitors, a non-limiting example of which includes Relatlimab, as well as combinations of ICI inhibitors, one non-limiting example of which is Relatlimab that is used in combination with nivolumab and is referred to as Opdualag, and any other ICI, such as anti-TIGIT and anti-TIM-3 agents.

The present disclosure is different from prior approaches in that the described biomarkers have not been previously shown to be predictive for responses to an immune checkpoint inhibitor or a combination of such inhibitors before treatment with an immune checkpoint inhibitor. For example, U.S. patent publication 20200347456 discloses biomarkers that include, for example, MTRNR2L8. However, this publication is focused on tumors and tumor infiltrating cells that are analyzed after treatment. Further, it is the post-treatment values that are used to select individuals for CDK4/6 treatment in combination with immune checkpoint inhibition. Accordingly, any resistance biomarker signature described in this publication is used for identification of cancer cells that have developed resistance to therapeutic agents as a consequence of treatment, e.g., oncogenic immune checkpoint resistances states. This publication also describes such resistance as an intrinsic property of tumor cells that arises only after immune checkpoint inhibition, and focuses on tumor microenvironment and its effect on tumor cells. This publication does not describe predicting responses by germline inherited genetics. This publication also does not describe predicting responses to combined immune checkpoint inhibition, nor does it describe any biomarkers, as a part of an immune checkpoint resistance signature, or having any connection to T cell status.

In marked contrast to the 20200347456 publication, and as described above, the present disclosure relates at least in part to characterization of haplogroups and their connection to ICI responses. This allows for establishment of a pre-treatment baseline value. This approach can be used to predict immune checkpoint resistance before an individual has ever received a checkpoint inhibitor, or for individuals who have stopped taking an immune checkpoint inhibitor and are under consideration for additional rounds of immune checkpoint inhibition. In connection with this, the data provided and discussed below demonstrate that resistance to immune checkpoint inhibition is a property of the host immune system at the baseline, a germline inherited genetic factor. Thus, the present disclosure does not require determining any biomarker from cancer cells or a tumor. Instead, the present disclosure is agnostic towards tumor status, and accordingly provides compositions and methods for predicting a priori those patients who should not be treated with a particular immune checkpoint inhibitor, and by identifying those patients, determining which individuals are candidates for immune checkpoint inhibition going forward. Thus, in examples, a sample tested using the compositions of this disclosure may be free of any sample of a tumor or cancer cells.

In examples, the disclosure provides for determination of a haplogroup and determining that the individual is a candidate to receive a particular ICI or combination of ICIs, or the individual should not receive or should discontinue a particular ICI or combination of ICIs. In one example, the method comprises determining that the individual has a haplogroup that is any of a K haplogroup, a T haplogroup, or a U haplogroup, and based upon this determination, determining that the individual will not likely sufficiently respond to a specific anti-PD-1 or anti-PD-L1 antibody, wherein either case the ICI is used as a monotherapy. Data presented in this disclosure demonstrate that individuals with haplogroup T are more sensitive to pembrolizumab and resistant to nivolumab. In examples, determining that an individual has the T haplogroup therefore indicates the individual should receive an ICI that is not nivolumab. In an example, an individual who has the T haplogroup is treated with pembrolizumab, or any other anti-PD-1 agent that is not nivolumab, and wherein the other anti-PD-1 agent that is not nivolumab elicits an improved anti-cancer response, relative to an actual or predicted response to nivolumab. The same applies to individuals with the K haplogroup and U haplogroup who show a similar trend to the T haplogroup. As such, individuals who are of the K, T or U haplogroup can be treated with a different ICI antibody or a combination thereof.

In examples, individuals who are of the K, T or U haplogroup may be treated with an ICI that is not anti-PD-1 or anti-PD-L1, or may include anti-PD-1 or anti-PD-L1 in combination with a different ICI, such as a combination of anti-PD-1 or anti-PD-L1 with anti-CTLA4, or any other different ICI, examples of which are discussed herein.

In an example, the disclosure provides for determination of an individual as having a J haplogroup and determining that the individual is a candidate for a particular ICI therapy, such as an anti-PD-1 or anti-PD-L1 therapy. The disclosure also includes assessment of L and other haplogroups that may be informative of ICI responses, and tailoring ICI treatments accordingly. The disclosure includes determining haplogroups using any one or combination of markers that are described herein by way of the haplogroup tables presented below.

In more detail, and without intending to be limited by any particular theory, this disclosure provides an analysis of the presence of novel surrogates of transcriptomic and genetic diversity of peripheral immune cells, as putative more personalized biomarkers of ICI efficacy. In particular, we determined whether mitochondrial haplogroups represent novel biomarkers predictive of ICI outcome. Using peripheral blood samples from a large metastatic melanoma clinical trial (CheckMate-067) with extensive clinical information harmonized across study sites, we assessed if the transcriptomic and genetic diversity of peripheral CD8+ effector cells associates with the response to ICI therapies (anti-PD1 single-line Nivolumab or “NIVO” and COMBO; treated by Ipilimumab or “IPI” and NIVO) and whether such changes link with patients' mitochondrial genetic repertoire. Subsequently, we validated the results in the standard of care setting using patients collected by the Germline Immuno-Oncology Melanoma Consortium (IO-GEM).

The following description is intended to illustrate but not limit the disclosure.

### Materials and Methods

### Study Population

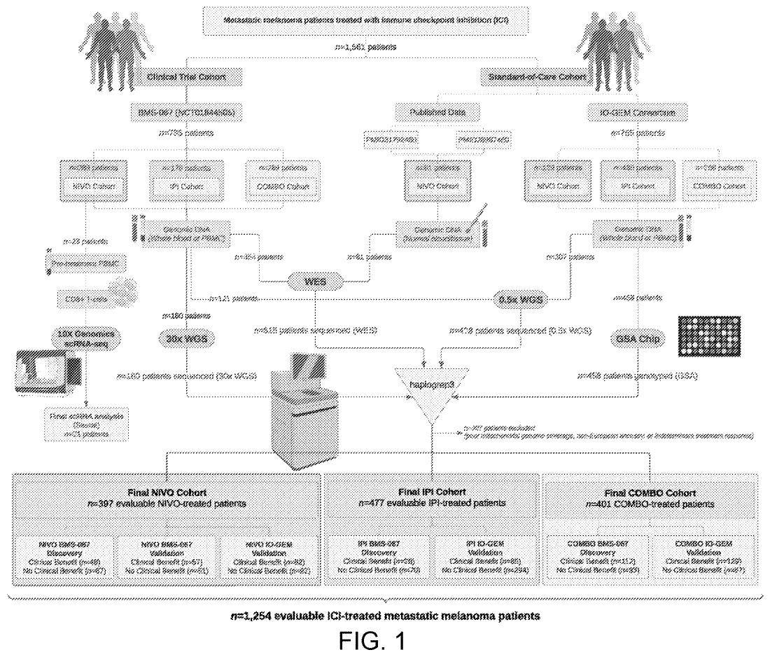

In this study, we used patient samples from the CheckMate-067 clinical trial (CM-067, ClinicalTrials.gov identifier NCT01844505), a 3-arm Phase III study that enrolled therapy-naïve advanced melanoma patients treated between 2013-2015 with single-agent ipilimumab (IPI), nivolumab (NIVO), or the combination (COMBO, IPI/NIVO) (FIG. 1).^{1,2} In the present CM-067 analysis, we sequenced pretreatment specimens of PBMCs or purified peripheral CD8+ T cells from n=155 NIVO and n=126 COMBO patients and utilized previously generated data from whole blood-based whole exome sequencing (WES) of patients treated by NIVO, COMBO, and IPI (n=454). In total, n=526 CM-067 patient samples were used in this analysis. The best overall response (BOR) to treatment in CM-067 was defined as complete response (CR), partial response (PR), stable disease (SD), or progression of disease (POD), based on Response Evaluation Criteria in Solid Tumors (RECIST) v.1.1.^{1,24} Clinical benefit (CB) was defined as those with CR, PR, or durable (d) SD (stable disease without progression for at least 6 months).²⁵ Patients with no clinical benefit (NCB) were defined as PoD or SD who progressed within 6 months (PSD). Overall survival (OS) and progression-free survival (PFS) were defined as described previously.¹

For validation of the findings from CM-067, we employed the Immuno-Oncology Germline Genetics in Melanoma (IO-GEM) Consortium, a standard-of-care (SOC) cohort of ICI-treated melanoma patients treated from centers in the US, Europe, and Australia. Samples for this analysis were collected the following institutions: New York University Langone Health (NYULH), Memorial Sloan Kettering Cancer Center (MSK), University of California Los Angeles's Jonsson Comprehensive Cancer Center (UCLA-JCCC), Massachusetts General Hospital (MGH), Dana Farber Cancer Institute (DFCI), University of Chicago Comprehensive Cancer Center (UCCCC), University of Colorado Cancer Center (CUCC), Roswell Park Cancer Institute (RPCI), and the National Tumor Institute Fondazione G. Pascale in Naples, Italy (INT-IRCCS). All patients had metastatic melanoma (MM) treated with NIVO, COMBO, or IPI (FIG. 1). Each patient provided blood samples (whole blood or PBMC) for DNA extraction. The sample and data collection protocols were approved by each institution's IRB and patient informed consent was obtained prior to collection. We also obtained data from publicly available sequencing of ICI-treated MM patients.^{26,27} In total, the SOC validation cohort consisted of n-675 patients. Response status was determined by the centers and was similarly dichotomized into CB (CR, PR, and dSD) and NCB (PSD and POD). Patients with SD for whom we could not determine the duration of their stable disease were removed from the analysis (n=5).

For prognostic analyses of survival stratified by haplogroups, we used a cohort of immunotherapy-naïve melanoma patients who were prospectively enrolled in the Interdisciplinary Melanoma Cooperative Group (IMCG) at NYULH from 2002-2018 (n=1,024 subjects).²⁸ Clinicopathological, follow-up, and biospecimen collection protocols for the IMCG have been previously described.²⁸⁻³⁰ For confirmatory analysis of the expected MT-HG population distribution, we obtained MT-HGs from n=420 participants from the European populations in the 1000 Genomes Project (1kGP).³¹ Genomic data for this analysis were collected from 1kGP Phase 1 (n=244 subjects) and Phase 3 (n=176 subjects).

### Sample Isolation

Pre-treatment PBMCs were used to isolate CD8+ T-cell populations from 240 patients. The CD8+ cells were negatively selected from the overall PBMC population using an Easy Sep Human CD8+ Enrichment Kit (STEMCELL Technologies, Vancouver, BC, Canada) per manufacturer's instructions. The RNA/DNA were extracted using the AllPrep DNA/RNA kit for simultaneous purification of genomic DNA and total RNA from human cells (Qiagen, Germantown, MD). For ATAC-seq, two ATAC-seq libraries per patient were generated from each patient to ensure data integrity. Each library preparation used ~50,000 viable CD8+ cells and was prepared as previously described.

### RNA and ATAC Sequencing and Data Pre-Processing

The rRNA-seq depletion has been used for preparation of RNA libraries, sequenced on the Nova-seq using 150-base paired-end sequencing to a depth of ~30 million reads per sample.

The SMARTer Stranded Total RNA-Seq Kit v2 Pico Input Mammalian (TaKaRa Bio USA, Mountain View, CA) was used to prepare rRNA-depleted RNA-seq libraries. Each library was then quantified by qPCR and libraries were pooled for sequencing using 150-base paired-end sequencing to a depth of ~30 million reads per sample.

Each library ATAC-seq library was quantified by qPCR and the libraries were pooled for sequencing on the Nova-seq using 150-base paired-end sequencing to a depth of ~30 million reads per sample.

### 30× WGS Sample Preparation and Data Processing

We isolated genomic DNA from pre-treatment PBMCs from the CheckMate-067 clinical trial using the Qiagen Blood and Tissue Kit (Qiagen, Germantown, MD). Libraries for whole genome sequencing (WGS) were prepared per manufacturers' instructions. Samples were prepared using the Illumina TruSeq DNA PCR-Free kit (for samples with DNA content >500 ng) or the New England BioLabs NEBNext® Ultra™ II DNA Library Prep Kit for Illumina (for samples with <500 ng DNA). Samples were sequenced to a target of 30x genomic coverage on an Illumina HiSeq X or NovaSeq 6000 per manufacturer's instructions, using 2×150 bp paired-end reads.

Sequencing FASTQ files were processed using the Genome Analysis Toolkit (GATK) workflow.³² Briefly, raw FASTQs were aligned to the Genome Reference Consortium Human Build 38 reference genome (GRCh38) using bwa mem (version 0.7.17).³³ Aligned Binary Alignment Maps (.bams) were merged using sambamba merge (version 0.6.8),³⁴ sorted with samtools sort (version 1.9),³⁵ and duplicates marked using sambamba markdup. We then performed two rounds of base quality score recalibration (BQSR) using GATK BaseRecalibrator (version 4.1.2.0). To identify the variants in the mitochondrial genome, Variant Call Format files (.vcfs) were generated by passing the aligned .bams to GATK's Mutect2 (version 4.2.1.0) using mitochondria-mode which is designed for sensitive variant detection at high sequencing depth. Finally, mitochondrial haplogroups were classified by passing the .vcfs containing mitochondrial variants to haplogrep3 (version 3.2.1).³⁶ To define the haplogroups, we used the mitochondrial phylogenetic tree from Phylotree (version 17.1).³⁷ aligned to the revised Cambridge Reference Sequence (rCRS) by specifying-tree phylotree-rcrs@17.1 in the haplogrep classify statement.

### Single-Cell RNA-Sequencing and Data Analysis

CD8+ T-cells were negatively selected from pre-treatment PBMCs from 23 NIVO-treated patients using the EasySep Human CD8+ Enrichment Kit (STEMCELL Technologies, Vancouver, BC, Canada) per manufacturer's instructions. Single-cell suspensions for each patient were individually tagged with unique hashtag antibodies (TotalSeq-B Anti-Human antibodies, Biolegend) and each sample was washed 3 times with 1×PBS+2% BSA+0.01% Tween before pooling. Four patient samples were pooled as one sample in suspension (n=~ 12,500 cells per hashtag, n=~ 50,000 total cells per pool) for a total of six pooled samples. Single cells were partitioned into Gel Beads-in-Emulsion (GEMs) using the Chromium Next GEM Chip G and tagged with a common 10× barcode and a unique molecular identifier (UMI) (10× Genomics, Chromium Next GEM Single Cell 3′ GEM Library & Gel Bead Kit). Reverse transcription, amplification, and library preparation of 3′ gene expression libraries were performed per published protocols (10× Genomics).³⁸ Libraries were sequenced using the NovaSeq 6000 platform at a minimum of 20,000 read pairs per cell at the Perlmutter Cancer Center Genome Technology Center Shared Resource (GTC).

Single-cell (sc) RNA-seq QC and downstream analysis of CD8+ T cells were performed with the Seurat v3 R toolkit.³⁹⁻⁴² Demultiplexing of samples based on hash tag oligos (HTOs) and annotation of doublets removed from the analysis was performed using “HTODemux” function with default parameters. Cells with gene counts >200 and <12,500 were kept for downstream analysis while genes expressed in <3 cells were filtered out. Cells expressing >25% mitochondrial genes and <0.05% ribosomal genes were discarded. 92,531 CD8+ T cells from 21 patients successfully passed QC and were used for the present analysis. Batch correction was then performed in Seurat using the integration method to mitigate artefactual differences between sequencing pools. Normalization of the data was performed using “NormalizeData” with the “LogNormalize” method and the most variable genes were found using the “Find VariableGene” function with “nfeatures” set to 2000. PCA was run on 50 dimensions and after a visual inspection of the elbow plot, the first 15 PCs were chosen to construct a Shared Nearest-neighbor (SNN) graph with the “FindNeighbors” function. “FindClusters” was applied to identify clusters with the resolution set to 0.5. The Uniform Manifold Approximation and Projection (UMAP) algorithm was used to visualize the clustering in 2D space.

### Infinium Global Screening Array Sample Preparation and Data Processing

Mitochondrial genotypes were determined for a subset of the IO-GEM SOC cohort using the Infinium Global Screening Array Multi-Disease (GSA-MD) platform, version 2.0 and version 3.0, which includes a drop-in panel of an additional ~50,000 multi-disease-associated genomic markers. Genomic DNA from these patients was isolated from whole blood or PBMCs using the Qiagen DNeasy 96 Blood and Tissue Kit (Qiagen, Germantown, MD). Using Qubit and gel electrophoresis, we quantified DNA concentration and assessed DNA integrity. Only high-quality samples with no evidence of degradation and a minimum concentration of 10 ng/μL were genotyped. Prior to genotyping, samples were identity-tracked using a panel of 25 previously curated variants43 using the Sequenom MassArray System (Agena Bioscience Inc., CA), per manufacturer's instructions. We observed high concordance (>99%) between the identity panel and the GSA-MD, minimizing the likelihood of possible sample mismatches.

Using Illumina's GenomeStudio (version 2.0), raw intensity data (.idat) files from the GSA-MD were converted to genotype calls with a GenCall score threshold of 0.15. We exported these genotype calls from GenomeStudio to PLINK^{44,45} format for downstream analysis using the PLINK Input Report Plug-in (version 2.1.4). Genotype quality assurance (QC) was performed in PLINK (version 1.9/2.0) by first excluding SNPs not present in ≥20% of the samples (assumed to be poorly genotyped SNPs) using the —geno command in PLINK. We then excluded any patients missing ≥20% of the remaining SNPs using the PLINK—mind command (assumed to be patients with poor overall genotype quality).

As haplogroups are defined by the specific nucleotide at each position, and not only whether a subject has a reference or an alternate allele, haplogrep3 requires the SNPs in the input .vcf to be reported according to the rCRS reference sequence. GSA probes can target sequences on either the forward or the reverse DNA strand; SNPs called from probes on the forward strand are reported according to the rCRS, while those on the reverse strand are reported as the complementary base. We therefore identified all the mitochondrial SNPs with GSA probes on the reverse strand and translated them to the complementary base using the—flip command in PLINK. Using the—exclude PLINK command, we additionally dropped several mitochondrial SNPs that were retained after the—geno filter but which are not well covered on the GSA. We then generated .bed files aligned to the GRCh37 reference genome using the —ref-from-fa PLINK command. While analysis can be run without aligning to a reference genome, PLINK's default behavior is to assign reference and alternate alleles according to the population frequency in the samples being analyzed, which may differ from the rCRS in many cases. Finally, we converted the .bed file to a .vcf using the —recode vcf PLINK command, and passed this to haplogrep3, including the—chip argument for sequencing array data.

### Ultra-Low-Coverage (0.5x) WGS Sample Preparation and Data Processing

Previously, we have shown that ultra-low-coverage WGS (lcWGS; defined as approximately 0.5x average genomic coverage) is comparable to both 30× WGS and GSA.⁴⁶ An additional 121 CheckMate-067 samples and 307 IO-GEM samples were sequenced using lcWGS. Genomic DNA was isolated as described above (see 30× WGS Sample Preparation). Sequencing libraries were prepared using either the NEBNext® Ultra™ II DNA Library Prep Kit for Illumina or the Illumina DNA Prep kit, including up to 8 cycles of PCR. Libraries were quantified using real-time PCR and sequenced on either an Illumina HiSeq X or NovaSeq6000 per manufacturer's instructions using 2×150 bp paired-end reads.

lcWGS FASTQs were processed into .bams according to the GATK workflow as described above (see 30× WGS Data Processing). Using Mutect2's mitochondrial mode, we generated .vcfs containing mitochondrial variants, which were passed to haplogrep3 as described above. To ensure concordance between the three sequencing modalities employed in this analysis, we compared the haplogrep3 results from each platform for a subset of patients. We observed excellent concordance between 30× and 0.5x WGS. 30× WGS and GSA also had very good concordance, however, one SNP defining a subgroup within Haplogroup V is not covered on the GSA, and therefore we are unable to detect this specific subpopulation. V Haplogroup patients are present in ~3% of the European population and have approximately 28 subpopulations, resulting in a potential misclassification rate of ~0.1% in the GSA data.

### Whole Exome Sequencing

Accurate mitochondrial genotypes can also be determined using reads from Whole Exome Sequencing (WES), even using probes designed exclusively for nuclear DNA.^{47,48} This is due to the increased content of mitochondrial DNA per cell (each cell contains hundreds of mitochondria, with each mitochondrion containing up to 10 copies of mtDNA),⁴⁹ which produces a high sequencing read-depth even among off-target reads arising from nuclear probes targeting nuclear mitochondrial sequences (NUMTs).

For additional CM-067 patients not included in lpWGS or WGS sequencing (n=454), we obtained off-target mitochondrial sequences⁴⁸ from pre-treatment matched-normal whole blood WES as described previously.⁵⁰ To obtain the MT-HG information, we processed the raw WES FASTQ files using the same pipeline as the WGS samples described above (see 30× WGS Data Processing). Because not all haplogroup-defining SNPs are covered by the targeted exome probes, we performed QC following haplogrep3 to remove samples with low-confidence MT-HG calls.

To determine mitochondrial haplogroups from our control population (using the 1000 Genomes Project), we obtained existing haplogroup data from Phase 1 participants⁴⁷ derived from off-target WES reads. We further obtained WES data from Phase 3 participants and derived haplogroup data as outlined below:

We additionally obtained low-coverage (2-4x) WGS from a subset of these patients and obtained haplogroup information as outlined above for our own lcWGS analysis. We confirmed 100% concordance between the haplogroups obtained from WES and lcWGS for these patients.

### Statistical Analysis

Differences in treatment outcomes by haplogroup were assessed using non-parametric chi-square tests, likelihood ratio tests (LRTs), and logistic regression models. LRT p-values are reported as they are robust to variations in sample size. We compared patients with CB versus NCB and analyzed NIVO-, IPI-, and COMBO-treated cohorts separately. In the IO-GEM cohort, we also conducted a sensitivity analysis assessing the association between MT-HG and patients with any NIVO treatment (NIVO as first- or second-line ICI therapy) and those with first-line NIVO ICI treatment only (ICI treatment naïve).

MT-HG phylogenetic trees were constructed using the R package “ape.”⁵¹ Forest plots for logistic regression odds ratios were plotted using the R package “gt.”⁵² Survival analysis was conducted using the R package “survival,”⁵³ with Kaplan-Meier curves calculated using the survfit function, and Cox Proportional Hazards Models generated using coxph. Pooled statistical models were adjusted for clinical parameters including age, sex, and disease stage when available. For a subset of patients for whom age was unknown (n=99), age was imputed based on the median age (61 years) for the overall cohort (n=1,254). Cox proportional hazard assumptions were confirmed using the Schoenfeld residuals test. A two-tailed p-value of <0.05 was considered statistically significant. All analyses were conducted in R version 4.2.2.

### Results

### RNA-Seq Gene Expression Analysis and ATAC-Seq Open Chromatin Analysis: CheckMate-067 (NIVO)-Treated Patient Cohort

RNA-seq was performed on the baseline peripheral CD8+ samples isolated from 70 single-agent NIVO patients constituting the discovery cohort. Differential expression analysis comparing CB (CR, n=14; PR, n=15) to NCB (SD, n=6; PD, n-35) identified 7 genes surpassing the FDR threshold for statistical significance (Table 1), with the most significant association observed for the transcript of HMAX, located in MTRNR2L8, a nuclear homolog of the mitochondrial peptide known as humanin (MTRNR2) (FDR adj. p-value 8.87E-09). The MTRNR2L8 sequenced can be accessed via Gene ID: 100463486. In the described data, HMAX was significantly associated with patients with NCB when compared to CB patients in NIVO-treated cohort (Table 1α). We optimized the quantitative resolution of the HMAX signal by refining the length of the RNA-seq reads. Without this optimization, the magnitude of the difference between CB and NCB at the HMAX locus is diminished.

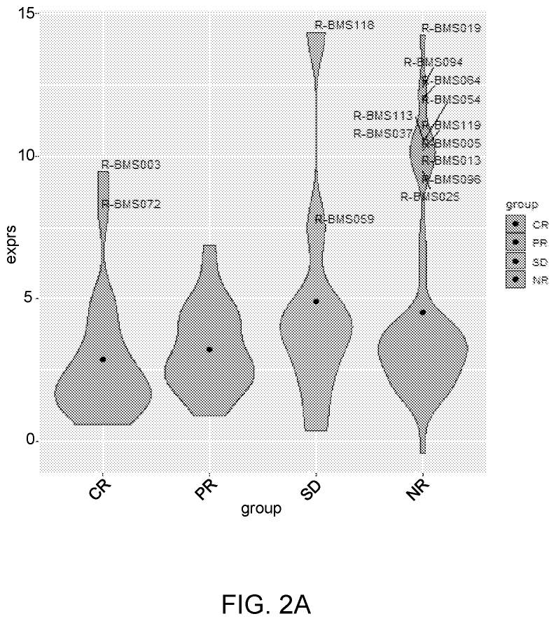

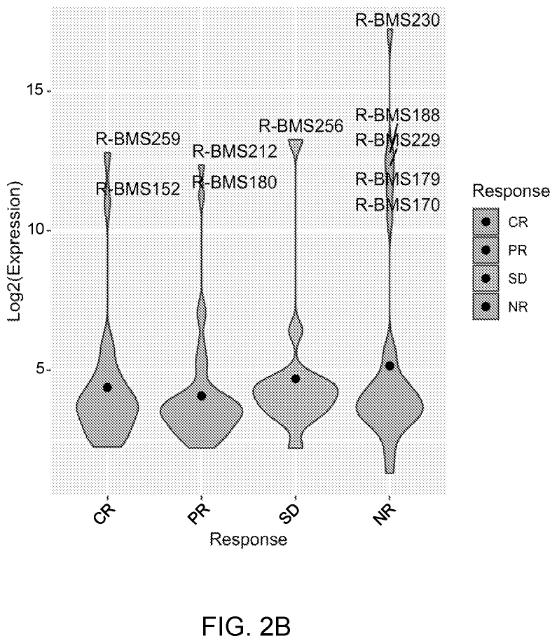

The presence of HMAX signal was also most significantly associated with NCB response in the validation group of additional 46 patients treated with single-agent NIVO (Table 1b) and the most significant association was observed in the pooled analysis of ALL NIVO-treated patients (n=116) (FDR adj. p=2.79E-13, log 2FC=4.03) (FIG. 2a, Table 1c).

We also performed independent RNA-seq analyses on baseline CD8+ samples from patients treated by COMBO (IPI/NIVO), and HMAX was the most significantly associated marker predictive of NCB in the discovery (FDR adj. p-value 2.00E-14, log 2FC=4.97), validation (FDR adj. p-value=1.40E-06, log 2FC=3.69) and pooled metanalyses (FDR adj. p=3.12E-28, log 2FC=4.95) (FIG. 2b, Table 2).

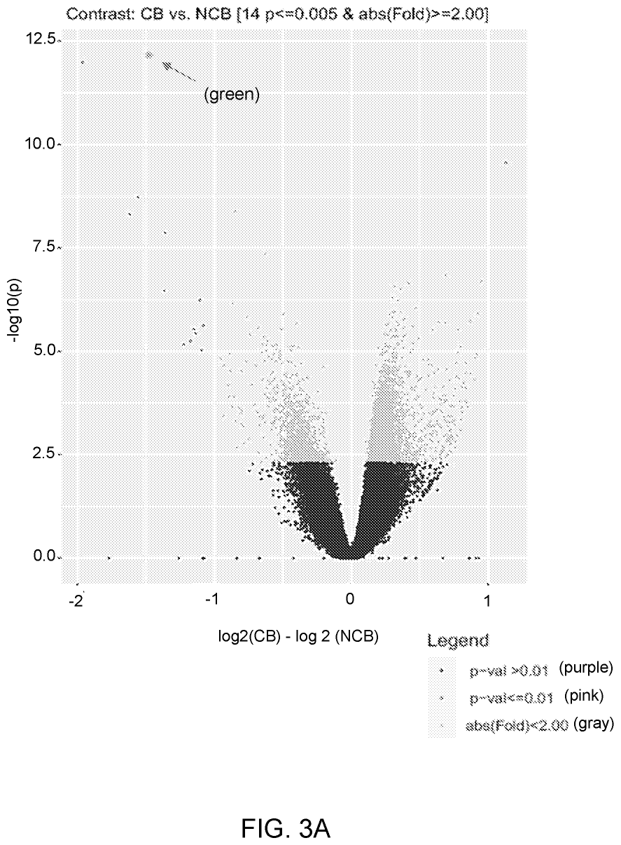

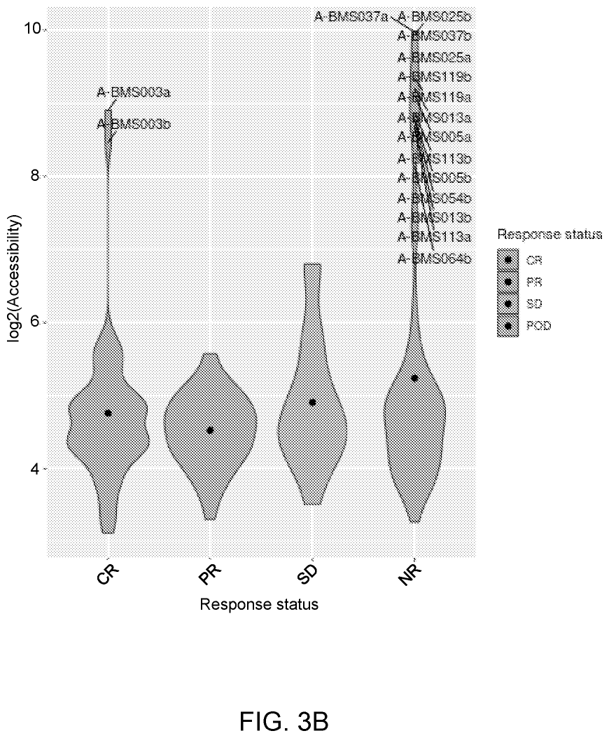

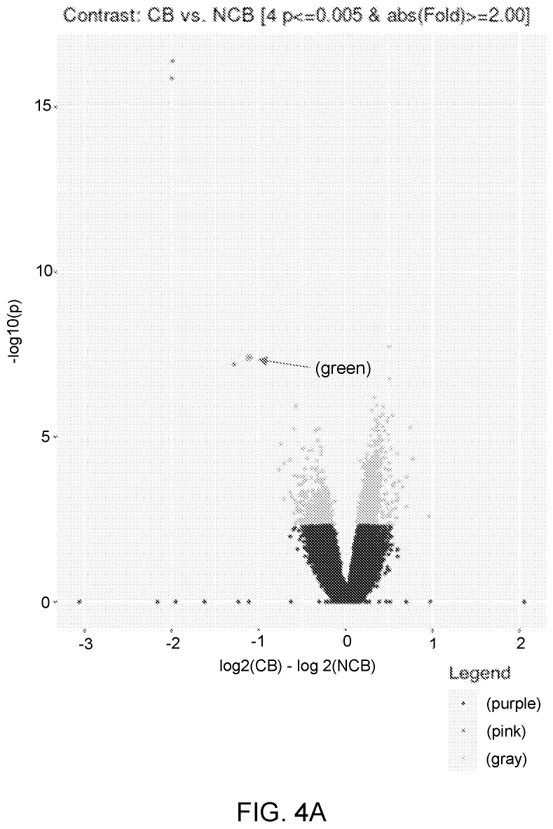

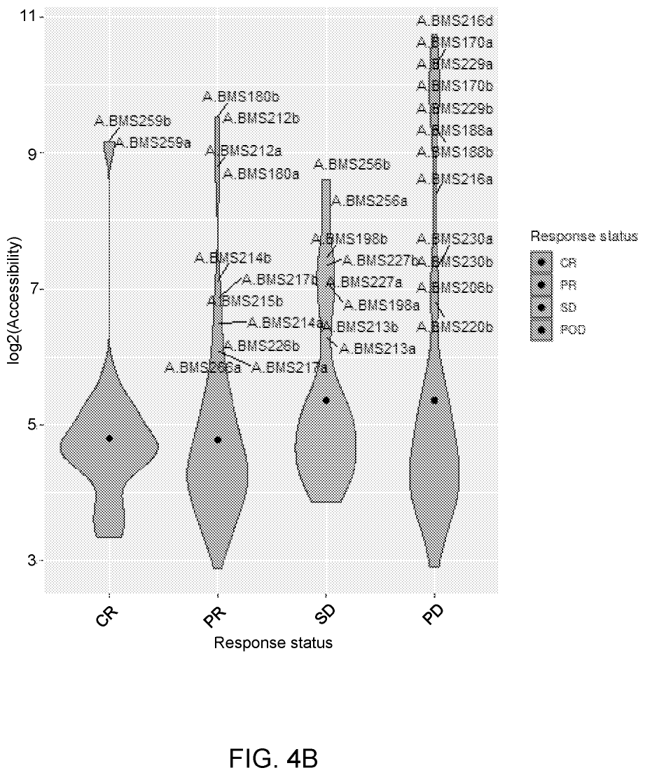

HMAX association with NCB resistant patients in NIVO and COMBO has been confirmed by independent interrogation of open chromatin accessibility using ATAC-seq analysis. In Both NIVO (FIGS. 3a-b, Table 3) and COMBO arm (FIGS. 4a-b, Table 4), HMAX showed significant association.

After detailed analyses of both RNA-seq and ATAC-seq data, the disclosure reveals that the sequencing reads corresponding to HMAX signal in both NIVO and COMBO patients are derived from mitochondrial genome of tested patients. While overall, CD8+-specific RNA-seq and ATAC-seq analyses have identified associations with HMAX and NCB, these differences are qualitative, rather than quantitative: the presence/absence of HMAX marker defines specific genetic variation in mitochondrial haplogroups. This finding led us to analyze mitochondrial genetic variability that would explain HMAX associations seen in RNA-seq and ATAC-seq analyses. Without intending to be constrained by any particular interpretation, it is considered that such a link with mitochondrial diversity has never been assessed before in the context of ICI or the host anti-tumor immune response.

### Genomic Analysis Discovery: CheckMate-067 NIVO

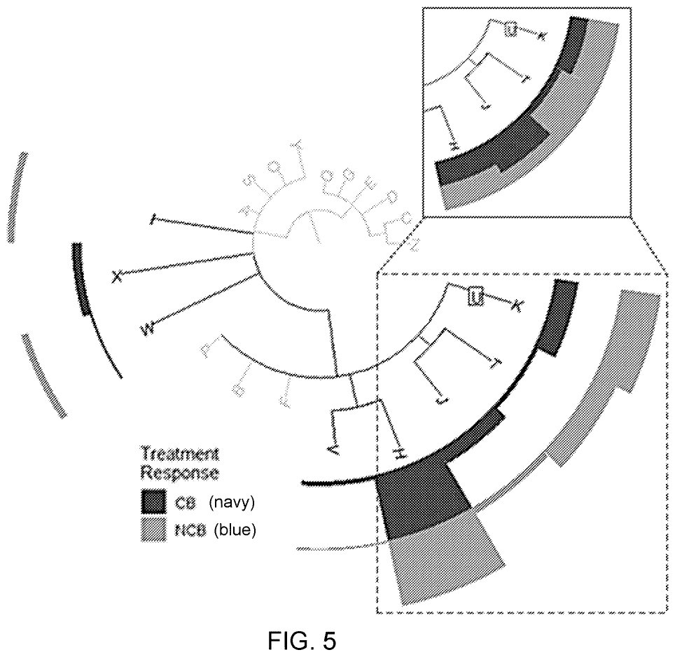

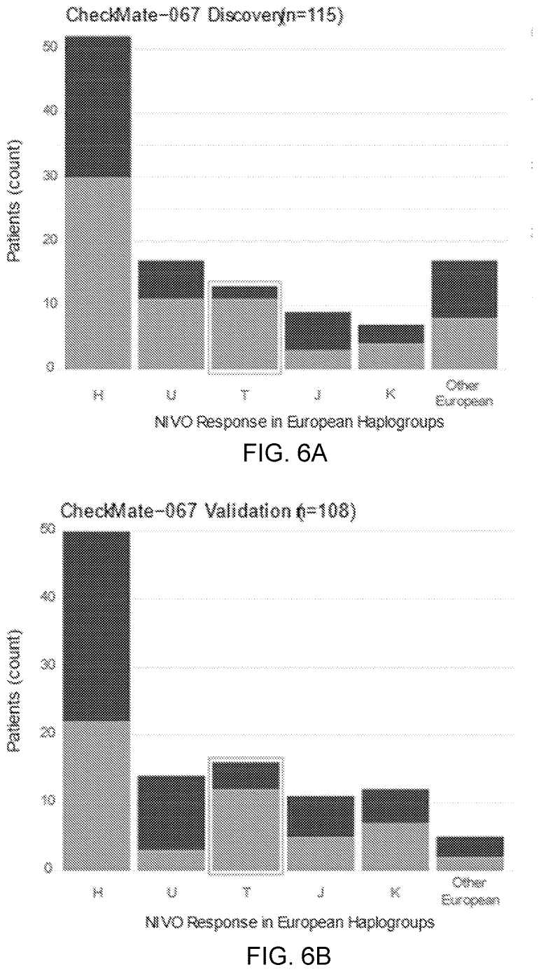

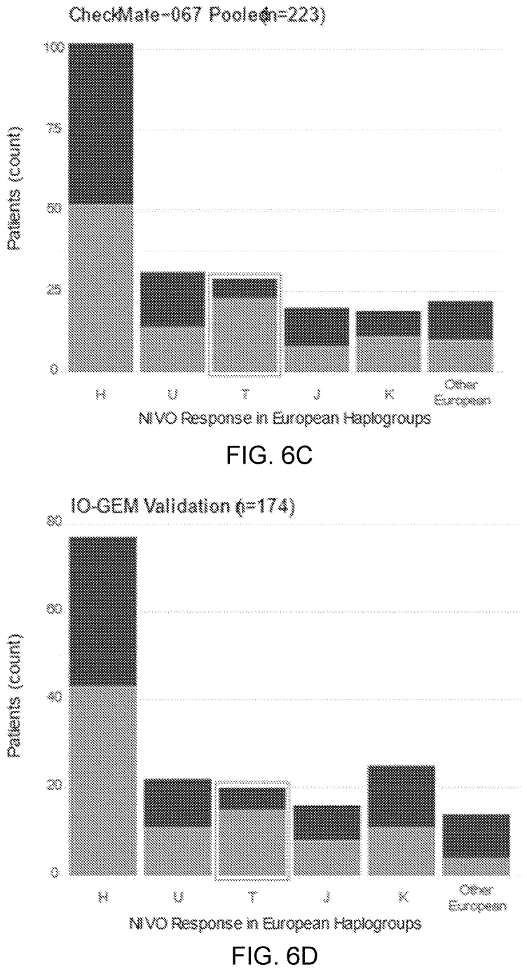

Given the HMAX association with MTRNR2L8, the disclosure provides a systematic analysis of the mitochondrial genetic diversity in the described studied population for their impact on ICI efficacy. As detailed in Material and Method section, we first used 30X WGS data from pre-treatment PBMCs from the CheckMate-067 clinical trial in NIVO cohort (Table 5). Applying the genetic analyses, as described, we observed that the distribution of haplogroups in the CheckMate-067 NIVO cohort is representative of the expected European population distribution (Table 6). Treatment response in H, the most common haplogroup in Europe (48%) and in CheckMate-067 NIVO (45%) shows 43% of patients experiencing clinical benefit (FIG. 5). However, when assessed individually, all other haplogroups have significantly different response rates than what is expected (FIG. 5). Some haplogroups are associated with poorer outcomes, such as T and K, while others, like J, appear to predict positive outcomes (FIG. 6a).

Of the major European haplogroups, we saw significant differences in NIVO efficacy for patients with the haplogroup T (HG-T) in the CM-067-D NIVO cohort (~10% of both the European and CM-067-D NIVO cohort) (FIG. 6a). Eighty-five percent of HG-T patients showed significantly worse NIVO efficacy compared to patients with all other haplogroups (p-value=0.04), with significantly higher odds of NCB (odds ratio (OR) for NCB, T vs. Other MT-HGs=4.52, (95% confidence interval (CI): 1.14-30.2)). To validate these associations, we performed further analyses in an additional n=108 NIVO-treated patients from the CM-067 trial (CM-067-V) (Table 5). We again found a strong association between NIVO NCB and HG-T (p-value=0.02), with a 75% NCB rate in HG-T patients (OR for NCB, T vs. Other MT-HGs=4.08 (95% CI: 1.31-15.5)) (FIG. 6b).

Combining all CM-067 NIVO patients (n=223) resulted in a highly significant association of NIVO resistance for HG-T patients (p-value=2.26E-03); compared to 48% CB in CM-067 NIVO, 79% of HG-T patients experienced NCB (OR for NCB, T vs. Other MT-HGs=3.99 (95% CI: 1.65-11.2)) (FIG. 6c). Adjusting the logistic regression model for age and disease stage further strengthened the association of HG-T with NCB (adjusted OR for NCB, T vs Other MT-HGs=4.19 (95% CI: 1.71-11.9)).

Because each haplogroup has a collection of SNPs that define it as described in tables of this disclosure, we sought to determine if there were any patients with private mutations in these canonical SNPs to see if they fit a similar response pattern. One patient in this cohort has the canonical SNPs associated with the K haplogroup but also has the mitochondrial SNP G1888A. This SNP is among those which define the T haplogroup, and this patient also does not respond to treatment, however, they do not have the other T SNPs.

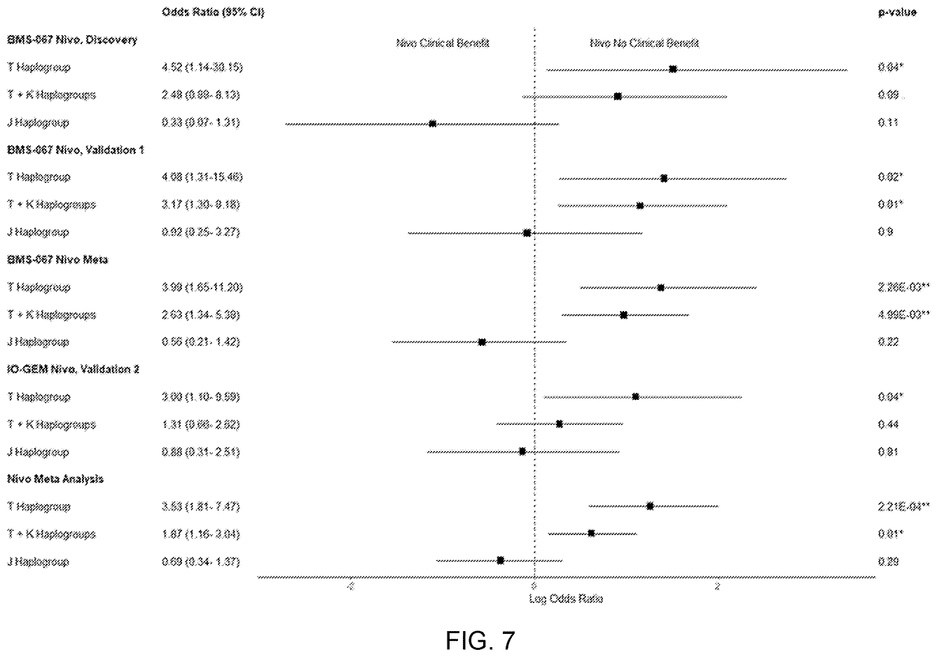

Other less common MT-HGs detected in both CM-067 and IO-GEM also showed suggestive associations with NIVO efficacy (FIG. 7). For example, patients with HG-K, similarly to HG-T, showed association trends with NIVO NCB consistently across all analytical stages, with the most significant associations observed in a meta-analysis when combined with HG-T. Conversely, HG-J patients showed suggestive associations with NIVO CB, but again not reaching statistical significance in every analytical stage. Hence, a larger more focused analysis will be needed to provide more definitive effect size estimates for these less common MT-HGs.

### Validation: IO-GEM NIVO

To further validate these findings outside of a clinical trial, in the standard-of-care (SOC) setting, we used samples obtained from the IO-GEM, a collaborative international consortium of immunotherapy-treated melanoma patients (n=129 NIVO samples). We also included data derived from previously published datasets (n=61).^{26,27} Clinical characteristics and MT-HG distribution of the final IO-GEM NIVO cohort (n=174) are consistent with those seen in the CM-067 analytical cohort (Tables 5-6). Overall efficacy in the IO-GEM NIVO cohort is similar to the CM-067 analysis, with 47% of subjects experiencing CB (FIG. 7d).

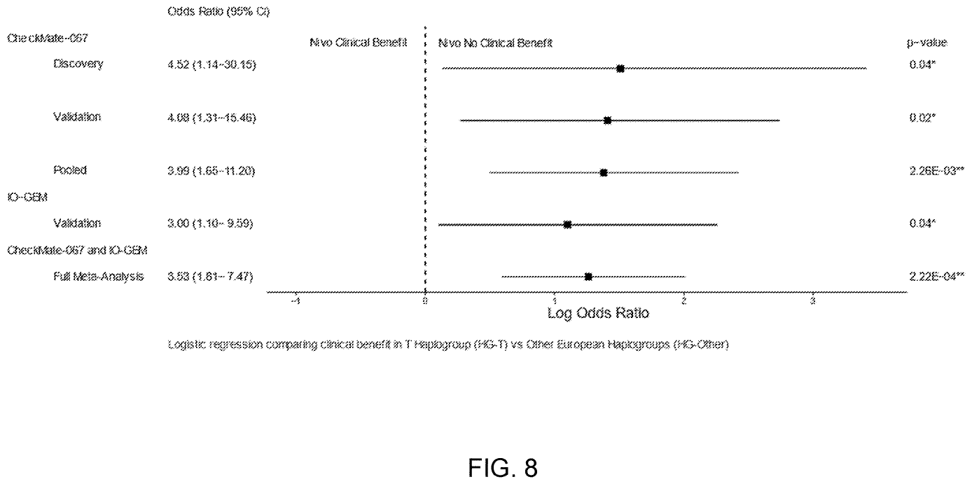

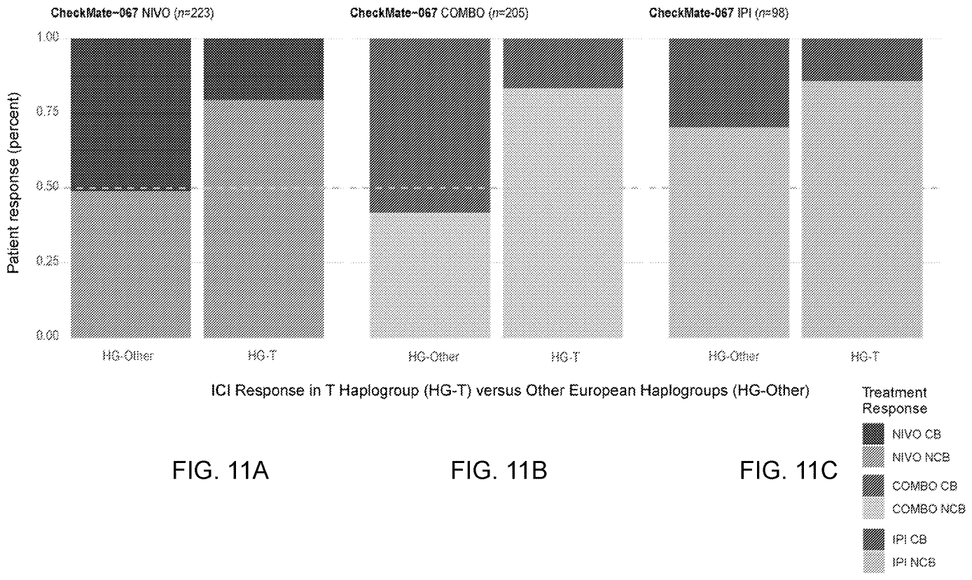

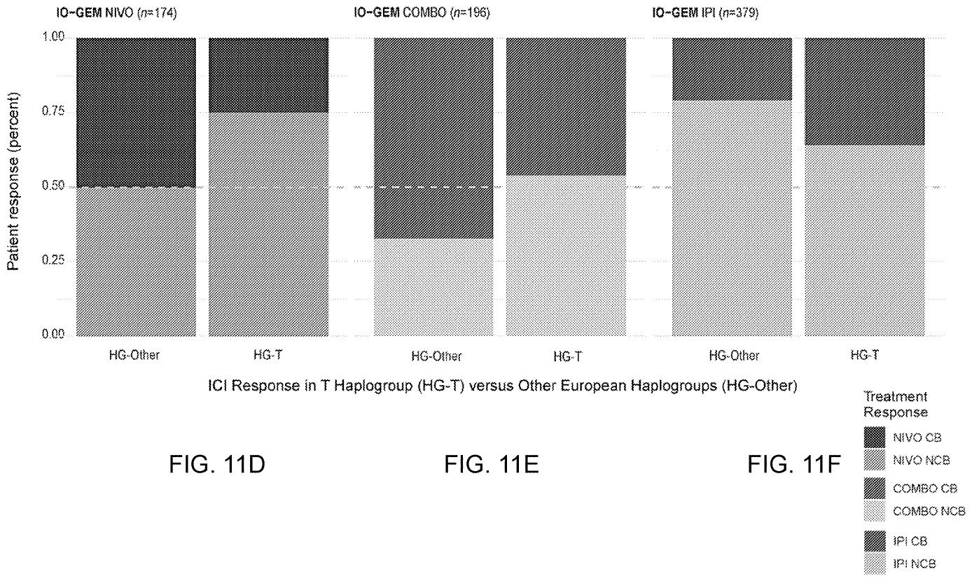

The IO-GEM analyses confirmed the observed associations from CM-067, showing that HG-T is significantly associated with NIVO resistance (75% NCB rate, p-value=0.04; OR for NCB, T vs Other MT-HGs=3.00 (95% CI: 1.10-9.59)). This association is comparable with the effect size and the significance observed in the discovery analysis in CM-067 (FIGS. 11a and 11d). A meta-analysis combining all NIVO-treated patients (CM-067 and IO-GEM, n=397) demonstrated a highly significant overall association with NIVO resistance (p-value=2.22E-04; OR for NCB, T vs Other MT-HGs=3.53 (95% CI: 1.81-7.47)) (FIG. 8), and the association remained significant after adjusting for age and sex (adjusted OR for NCB, T vs Other MT-HGs=3.50 (95% CI: 1.77-7.45)).

scRNA-seq

Given the growing evidence that mitochondrial metabolism impacts T cell differentiation,^{15,16,21,23,54} the disclosure provides an analysis of whether peripheral effector CD8+ T-cells, as the targets of anti-PD-1 therapy, show distinct phenotypic features in patients with NIVO-resistant MT-HGs (HG-T, HG-K) versus those of other haplogroups. We performed single-cell RNA-seq analysis (scRNA-seq) on CD8+ cells isolated from CM-067 NIVO-treated patients (n=21), which included 8 patients with CB (n=7 CR, n=1 PR) and 13 with NCB (n=13 PoD). To delineate the phenotypic differences associated with MT-HGs, we enriched the patient selection as follows: for NCB patients, 7 were selected with NIVO-resistant MT-HGs (NCB-HGR) (n=6 HG-T, n=1 with HG-K), and 6 with other MT-HGs (NCB-HG^{O}); for CB patients, 1 with HG-T and 7 with other MT-HGs.

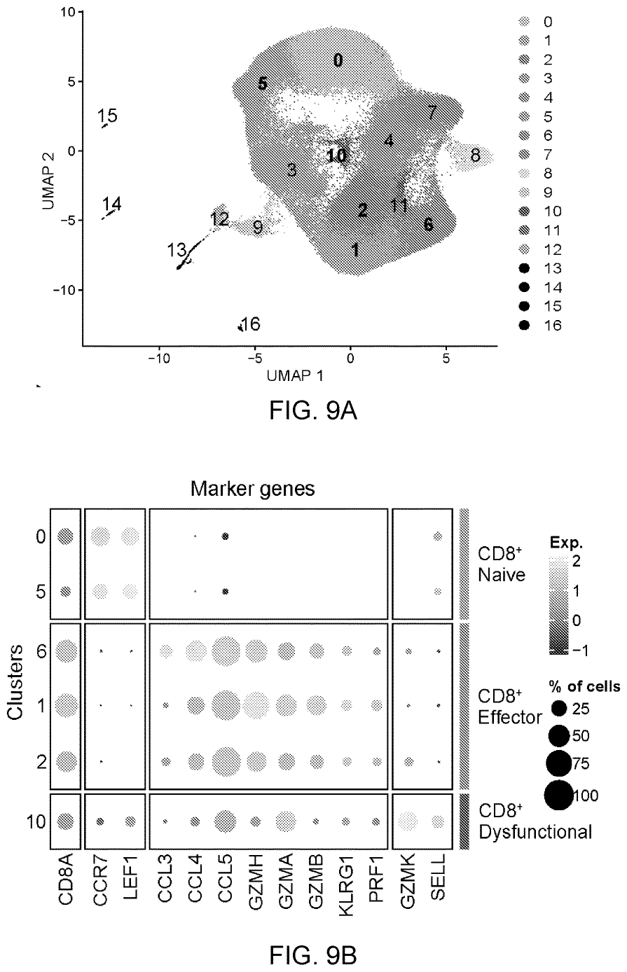

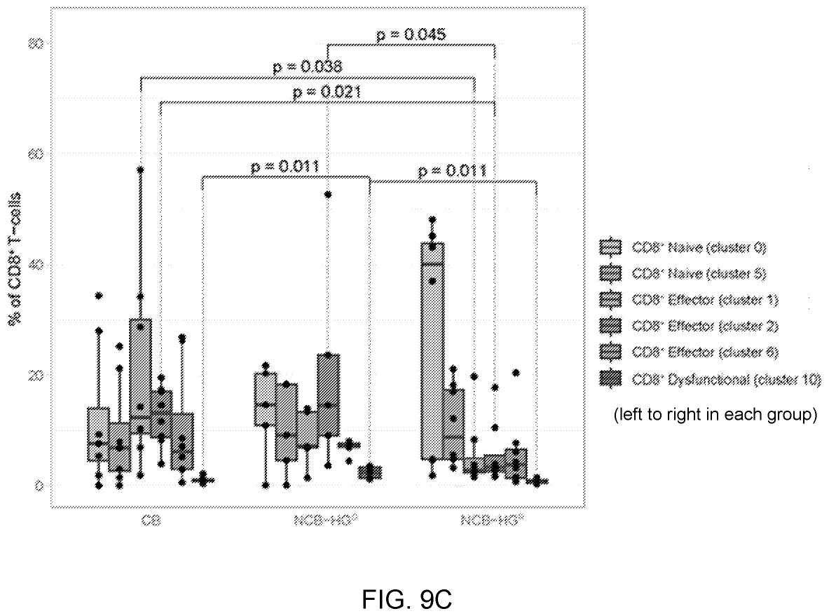

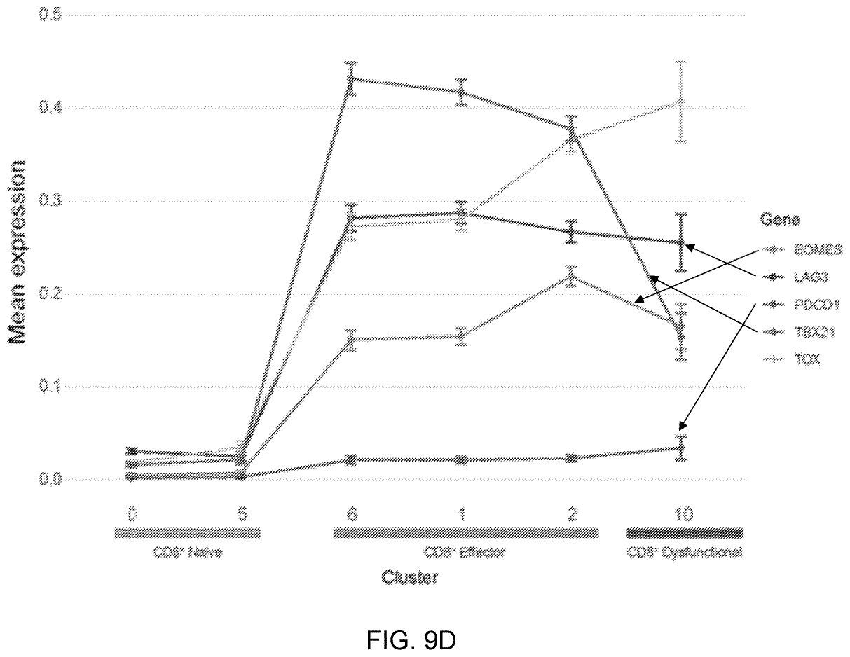

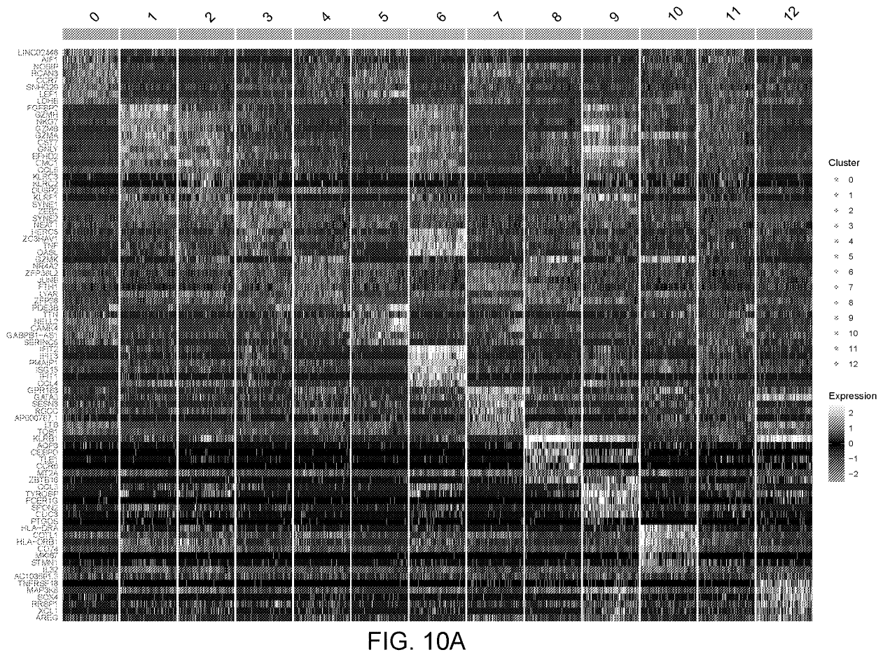

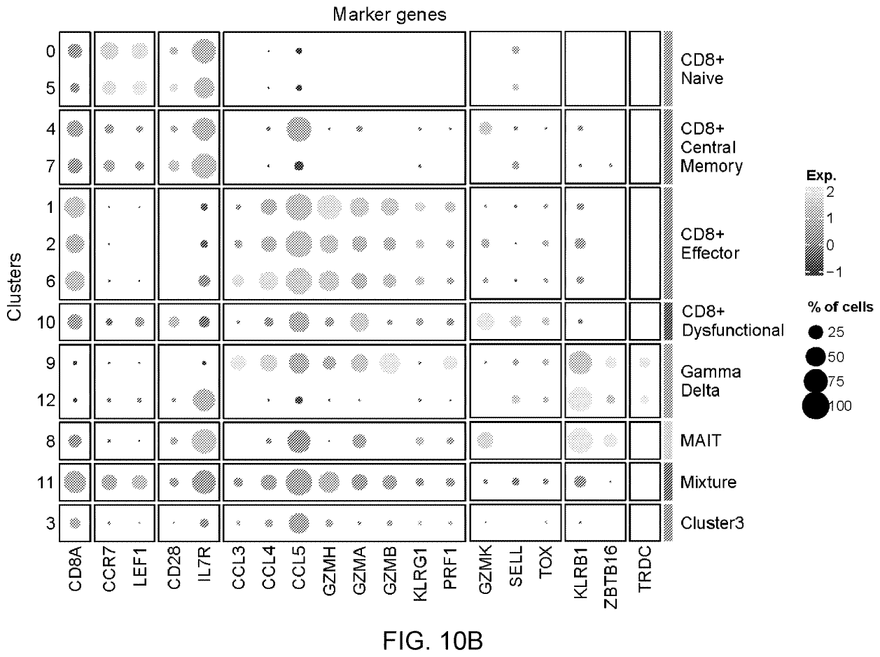

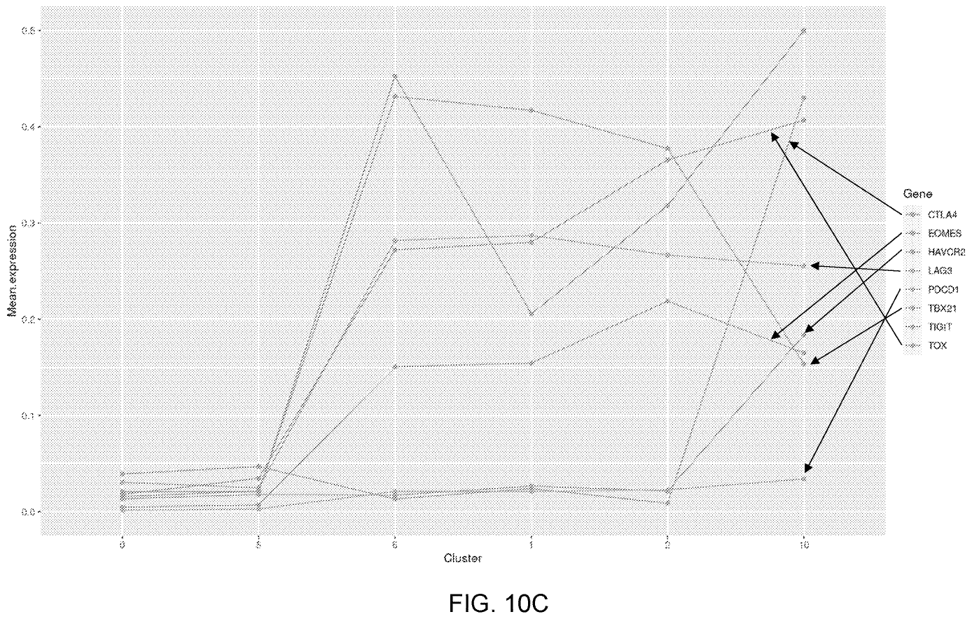

Using scRNA-seq pipelines,³⁹⁻⁴² we mapped the transcriptional profiles of the CD8+ cells using the Uniform Manifold Approximation and Projection (UMAP) algorithm and constructed a shared nearest neighbor (SNN) graph to identify 16 unique clusters (FIG. 9a). Based on differential gene expression profiles of T cell marker genes derived from established reference sets,^{42,55-57} we annotated these 16 clusters to specific cell types (FIGS. 10a-b). We defined major phenotypic states used for downstream analysis, ordering them by T cell differentiation state (FIG. 9b): naïve cells (cluster 0 and 5), effector cell cluster 6 (earliest stage of effector cell differentiation), cluster 1 (early exhaustion), and cluster 2 (late exhaustion), followed by dysfunctional T cells (cluster 10), defined as those with the highest mean expression levels of exhaustion markers (FIG. 10c).

When stratifying the CD8+ T cell phenotypes by NIVO response status and MT-HG, NCB-HG^{R} patients tended to have predominantly naïve cells and significantly fewer early exhausted cells (cluster 1, p-value=0.02) and late exhausted cells (cluster 2, p-value=0.04), compared to CB patients. Compared to NCB-HG°, NCB-HGR patients not only had fewer late exhaustion cells (cluster 2, p-value=0.05) but also significantly fewer dysfunctional cells (cluster 10, p-value=0.01) (FIG. 9c).

It has been shown that PD-1 inhibition can reinvigorate exhausted T cells and promote their effector function, however, only a subset of exhausted cells, characterized by high TBX21 (TBX21HI) and low EOMES (EOMESLOW), are capable of reinvigoration.^{58,59} Based on this model, we further refined exhausted phenotypes identified in our data and defined TBX21^{HI} exhausted cells, the primary target of reinvigoration by NIVO, and those with high EOMES (EOMESH) and/or high TOX (TOXHI), terminally exhausted cells that cannot be reinvigorated.^{57,60-64} Cluster 1, with TBX21^{HI} and EOMESLOW expression (FIG. 9d), likely represents the early exhaustion phenotype that can be reinvigorated and is predominantly present in CB patients. NCB-HG° patients show elevated levels of both cluster 2 (EOMES^{HI}, TOX^{HI}, and TBX21^{LOW}) and cluster 10 (EOMES^{LOW}, TBX21^{LOW}, and TOX^{HI}) (FIG. 9d), both likely the terminally exhausted cells with no capacity for anti-PD-1 reinvigoration. In contrast, NCB-HGR patients show a significantly lower proportion of all three states of exhausted cells, exhibiting a distinct baseline peripheral cell phenotype characterized by an overall lower fraction of differentiated CD8+ T cells in patients with these haplogroups.

Prediction of Clinical Efficacy by MT-HG Status with Other Immune Checkpoint Regimens

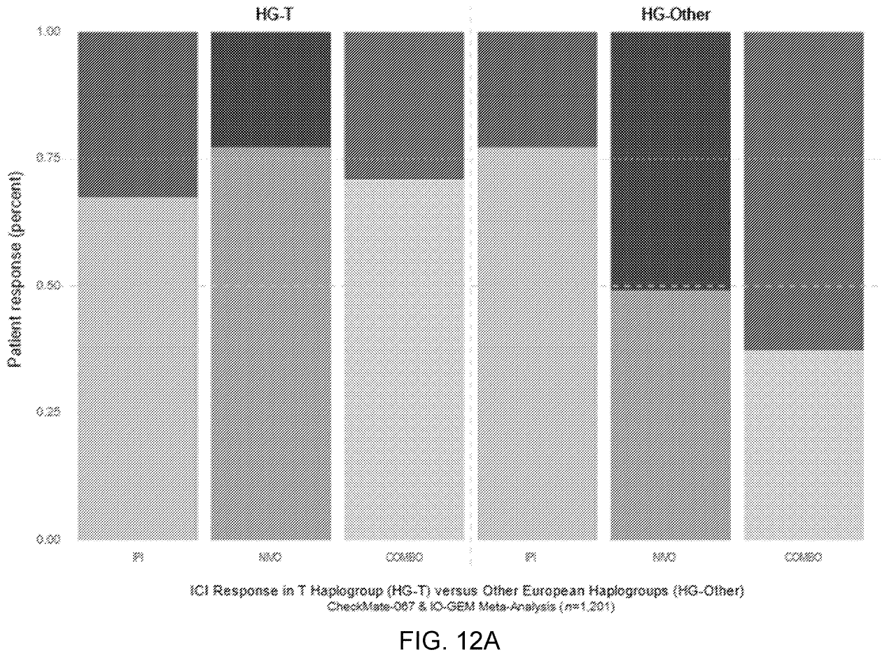

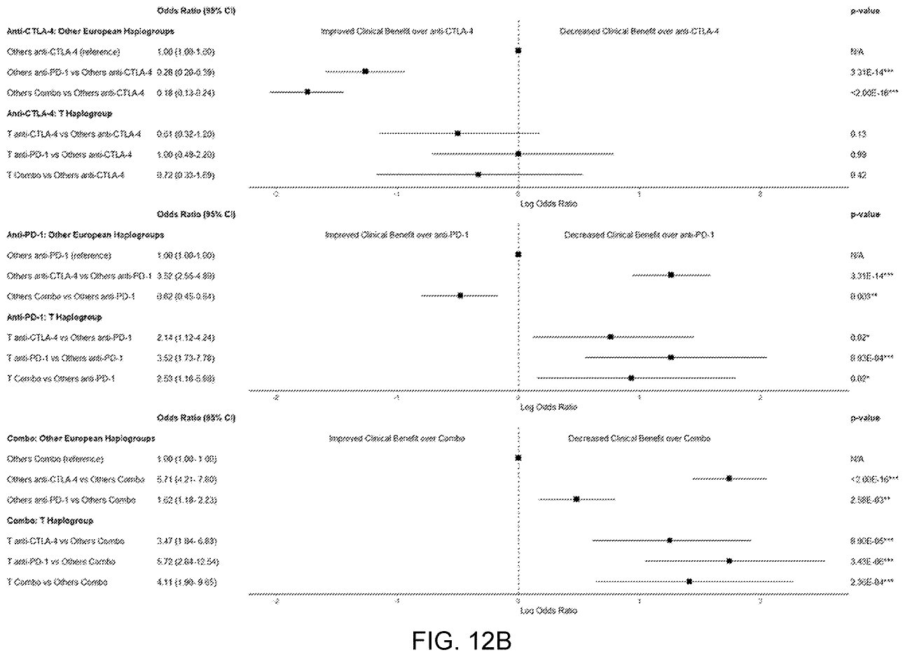

To evaluate whether MT-HGs associate with outcomes of other frontline ICI therapies in MM, we tested MT-HG associations with the clinical efficacy of combination IPI and NIVO (anti-CTLA-4/anti-PD-1; COMBO), which is currently an approved standard of care first-line ICI regimen for patients with MM. In CM-067, COMBO-treated HG-T patients showed significantly worse efficacy compared to all other MT-HGs (p-value=7.05E-04), with an 83% NCB rate (OR for NCB, T vs Other MT-HGs=6.99 (95% CI: 2.21-30.9)) (FIG. 11b). The same trend was shown in the IO-GEM COMBO cohort, with HG-T patients showing the poorest outcomes after COMBO (54% NCB vs 33% NCB in other MT-HGs) (p-value=0.12; OR for NCB, T vs Other MT-HGs=2.39 (95% CI: 0.76-7.73)) (FIG. 11e). Combining COMBO-treated patients from both CM-067 and IO-GEM showed a highly significant association (p-value=4.43E-04), with a 69% NCB rate in HG-T patients compared to 37% NCB rate in the other MT-HGs (OR for NCB, T vs other MT-HGs=3.73 (95% CI: 1.76-8.45)) (FIG. 12a). Adjusting the COMBO meta-analysis for age and sex demonstrated the same trend (adjusted OR=3.67 (95% CI: 1.71-8.41)).

We also tested whether MT-HGs are associated with single-agent IPI (anti-CTLA-4). The meta-analysis combining both CM-067 and IO-GEM (n=477) found no statistically significant association of IPI efficacy with any of the MT-HGs (p-value=0.13; adjusted OR=0.59 (95% CI: 0.31-1.18)) (FIG. 12a). Overall, our data show that patients with HG-T have comparably poorer outcomes in both NIVO and COMBO, the ICI treatment regimens with the highest efficacy in the general MM population (FIG. 12b).

### Association Between MT-HG and ICI Progression-Free (PFS) and Overall Survival (OS)

As survival is the most definitive indicator of ICI therapy success, we tested whether MT-HGs are associated with PFS and OS after ICI, using well-harmonized data from CM-067. We found significantly decreased PFS among the HG-T NIVO-treated (age, sex, stage adjusted PFS HR=1.91 (95% CI: 1.20-3.06), p-value=0.007) as well as COMBO-treated patients (adjusted PFS HR=1.98 (95% CI: 1.05-3.73), p-value=0.04). 80% of all HG-T patients progressed within the first four months of treatment, and their overall survival (OS) was also significantly poorer than patients who experienced CB (NIVO adjusted p-value <0.001; COMBO adjusted p-value<0.001), and comparably poor to all other patients with NCB. These data show that HG-T patients are less likely to achieve a durable clinical response from NIVO-based ICI (both single line and combination) compared to the other haplogroups.

### Evaluation of MT-HGs as an ICI-Predictive Outcome Biomarker Independent of Disease Prognosis

Given the observed associations of HG-T with NIVO and COMBO resistance, validated in multiple patient populations in this disclosure, we sought to evaluate whether MT-HG status is predictive of ICI outcomes or a reflection of overall disease prognosis. We assessed the association of MT-HGs and OS in a prognostic cohort of 1,024 ICI-naïve melanoma patients (not treated by ICI) enrolled at NYULMH,²⁸ with extensive follow-up. In this analysis, we found no difference in survival in early-stage (I-II) patients (n=915, p-value for OS by haplogroup=0.41) nor later-stage (III) patients in both univariable and multivariable analyses (n=109, p-value for OS by haplogroup-0.78). These data indicate that MT-HG status does not determine disease prognosis, but rather is an independent predictor of ICI treatment efficacy.

### Evaluating Response to Different Anti-PD-1 Monoclonal Antibodies by MT-HG

The present identification of a genomic biomarker of anti-PD-1 resistance has particular clinical significance given that single-agent anti-PD-1 treatments typically provide substantially improved clinical benefits and reduced toxicities compared to Ipilimumab (IPI), an ICI targeting the CTLA-4 receptor.⁶⁵ As anti-PD-1-based ICIs have now become frontline therapies in MM and other tumor types, identifying patients that are resistant to these treatments not only has important clinical implications for patient stratification to alternative regimens but may also aid in better biological understanding of anti-PD-1-based ICI resistance.

Two monoclonal antibodies (mAbs) targeting the PD-1 receptor are currently used in SOC: Nivolumab (NIVO) and Pembrolizumab (PEMBRO). Studies directly comparing the efficacy of the two drugs have found no statistical differences in melanoma^{66,67} or other cancers for which they are used, such as non-small cell lung cancer.⁶⁸ While this has led to a general consensus that there is no practical distinction between the two drugs, with both showing comparable efficacy in advanced malignancies, differences do exist in the structure of the NIVO and PEMBRO mAbs. These differences largely affect the mechanism by which they bind PD-1, with NIVO interacting primarily with the N-terminal loop^{69,70} and PEMRBO predominantly interacting with the C′D loop of PD-1.^{69,71} However, whether these structural differences impact response to treatment in individual patients, rather than in aggregated cohorts, has never been tested.

While we initially assessed the ICI-haplogroup association only in SOC patients treated with NIVO-based ICI, we also sought to identify if different MT-HGs showed comparable response to PEMBRO and/or whether the association with ICI efficacy and MT-HGs is anti-PD-1 mAb-specific. Using additional genomic data obtained from MM patients treated with SOC PEMBRO, we compared the association between mitochondrial haplogroups and efficacy for PEMBRO and NIVO-based ICI treatments.

We collected clinical and genomic data from 1,497 metastatic melanoma patients treated with first-line ICI to assess differences in anti-PD-1 ICI treatment efficacy by mitochondrial haplogroup (MT-HG). In particular, we sought to assess differences in outcome for haplogroup T (HG-T), which we have previously shown to be associated with significantly poorer outcomes to NIVO-based therapies, as discussed above. Overall, the cohorts of patients treated by the PD-1 inhibitors Nivo and Pembro are demographically comparable, with similar distributions in age and sex (Table 7) and similar MT-HG distribution was observed in the reference population from the 1000 Genomes Project (Table 8).

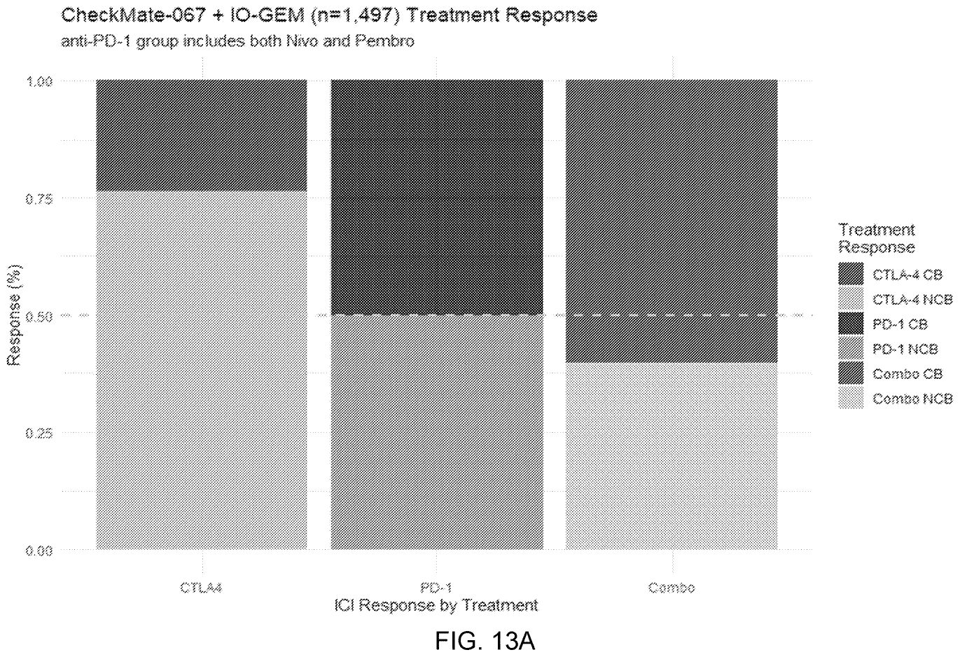

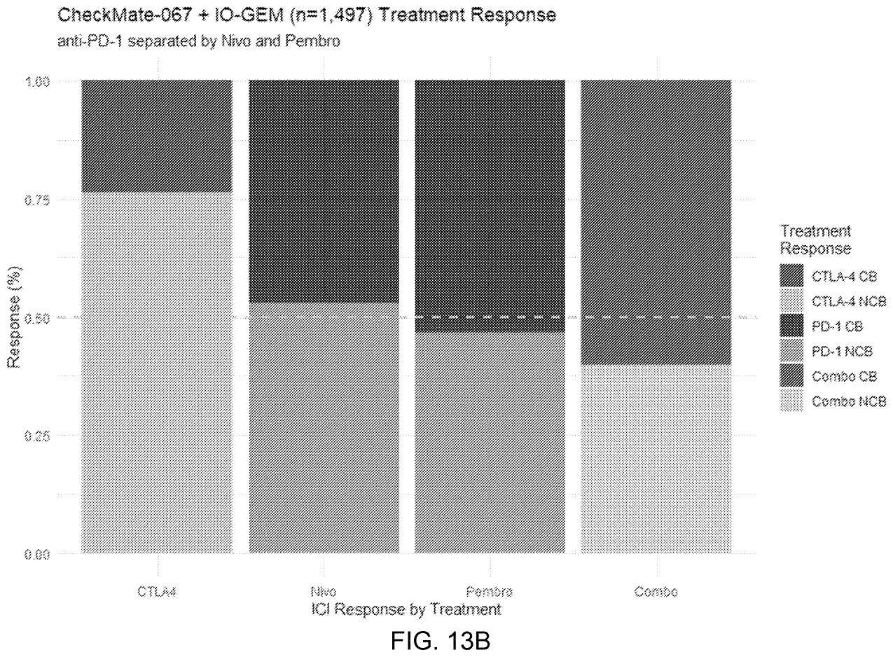

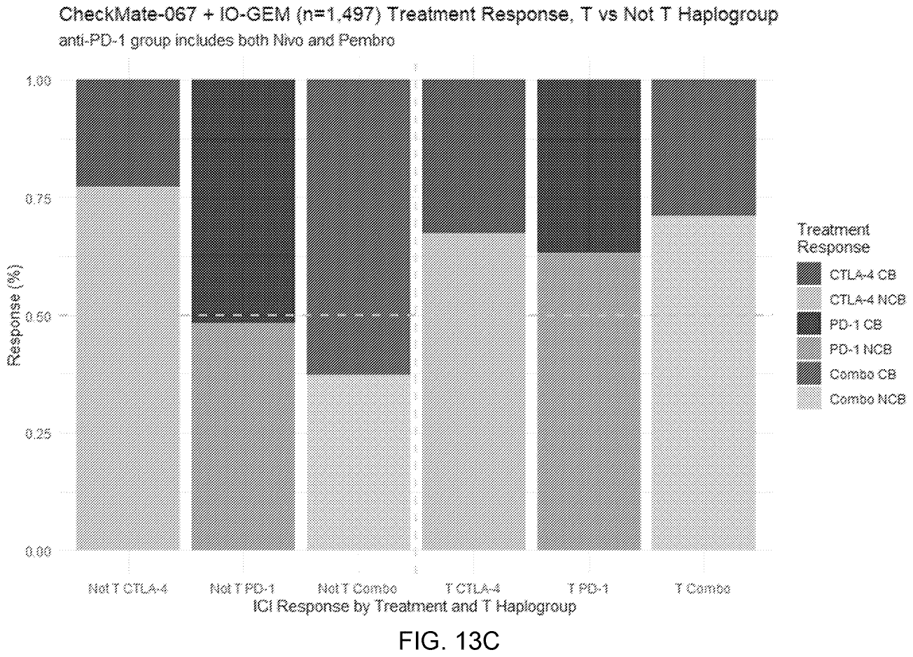

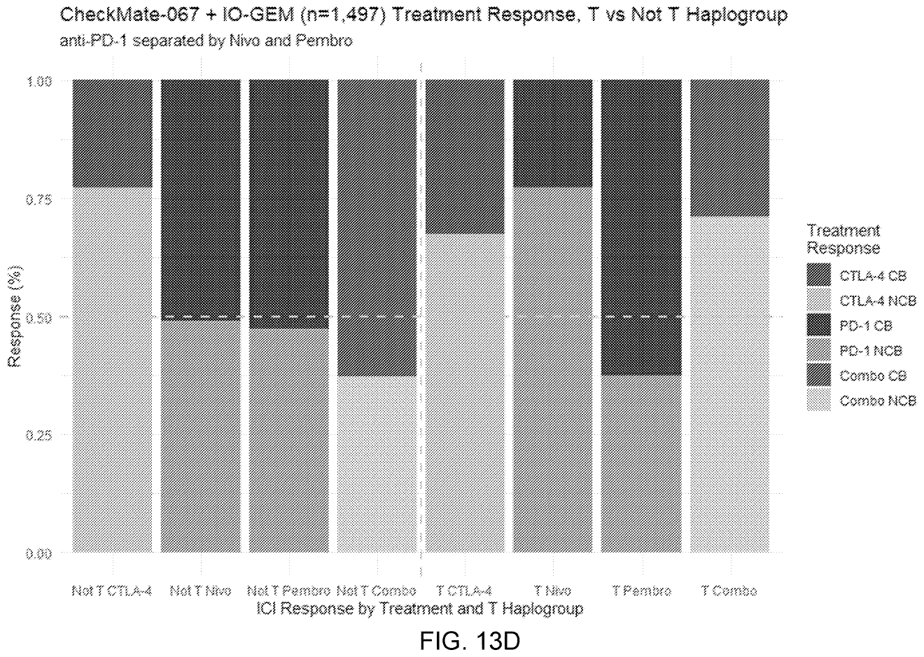

In the aggregate, assessing each checkpoint inhibition treatment as one group, clinical benefit (CB) for single-agent anti-CTLA-4 (IPI) was 24% CB, anti-PD-1 (inclusive of both NIVO and PEMBRO) was 50% CB, and combined anti-CTLA-4/anti-PD-1 (COMBO, consisting of IPI/NIVO only) was 60% CB (FIG. 13a, Table 9). These differences in treatment outcomes were statistically significant in both univariable and multivariable analyses (adjusted for age and sex; multivariable odds ratios (ORs) and p-values are reported). We observed significantly increased efficacy in anti-PD-1 compared to anti-CTLA-4 treatment (OR for NCB, IPI vs. anti-PD-1:3.27 (95% CI: 2.51-4.28), p-value <2.00E-16), as well as improved efficacy in COMBO compared to either anti-CTLA-4 (OR for NCB, IPI vs COMBO: 5.04 (95% CI: 3.77-6.79), p-value <2.00E-16) or anti-PD-1 alone (OR for NCB, anti-PD-1 vs COMBO: 1.54 (95% CI: 1.19-2.00), p-value=9.80E-04).

When assessing NIVO and PEMBRO anti-PD-1 treatments separately without stratifying by MT-HGs, the results are similar, with 47% CB in NIVO and 53% CB in PEMBRO (OR for NCB, NIVO vs PEMBRO: 1.22 (0.88-1.67), p-value=0.23) (FIG. 13b, Table 10). IPI outcomes were significantly worse compared to either NIVO (p-value=1.14E-11) or PEMBRO (p-value <2.00E-16), and COMBO outcomes were significantly better (COMBO vs NIVO p-value=5.65E-04, COMBO vs PEMBRO p-value=0.04).

When separating patients by MT-HG, we find that HG-T patients have significantly poorer outcomes when treated with anti-PD-1 (both NIVO and PEMBRO) compared to the other MT-HGs (p-value=0.02), with 37% CB for HG-T compared to 52% CB in other MT-HGs (FIG. 13c, Table 11). This is consistent with our previous analysis showing that HG-T had poor outcomes to NIVO-based therapies compared to other MT-HGs, with the worst outcome observed in the single-agent NIVO cohort (23% HG-T NIVO CB). However, when combining NIVO and PEMBRO into one anti-PD-1 treated cohort, HG-T patients have slightly better outcomes to anti-PD-1 monotherapy (37% CB) compared to IPI monotherapy (33% CB) or COMBO (29% CB). Although these distinctions are not statistically significant (p-value=0.67 and 0.58 comparing HG-T anti-PD-1 to IPI and COMBO, respectively), this is in contrast the described findings showing HG-T patients had the worst outcomes with NIVO monotherapy.

These underlying differences are explained when assessing the association of HG-T with NIVO and PEMBRO efficacy separately by each treatment. While the difference between NIVO and PEMBRO outcomes in the other MT-HGs is negligible (OR for NCB, NIVO vs PEMBRO in other MT-HGs: 1.01 (0.72-1.41), p-value-0.98), HG-T patients have significantly poorer outcomes when treated with NIVO compared to PEMBRO (OR for NCB, NIVO vs PEMBRO in HG-T: 5.43 (1.87-16.9), p-value=2.40E-03) (FIG. 13d, Table 12). While there is no statistical difference in HG-T outcomes between NIVO and IPI or COMBO (p-value=0.30 and 0.45, respectively), HG-T patients treated with PEMBRO respond significantly better compared to both IPI (OR for NCB, IPI vs PEMBRO in HG-T: 3.31 (1.20-9.67), p-value=0.02) and COMBO (OR for NCB, COMBO vs PEMBRO: 3.62 (1.19-11.8), p-value=0.03).

While in other MT-HGs the response to COMBO treatment is significantly better than either NIVO (p-value=3.54E-03) or PEMBRO (p-value=4.41E-03), not only is there no difference in outcome between other MT-HGs treated with NIVO or PEMBRO compared to HG-T treated with PEMBRO (p-value-0.36 and 0.37, respectively), but there is also no difference between outcomes for HG-T treated with PEMBRO compared to other MT-HGs treated with COMBO (OR for NCB, other MT-HG COMBO vs HG-T PEMBRO: 0.93 (0.40-2.28), p-value=0.87) (FIG. 13d, Table 12).

In order to develop a potential biological hypothesis underpinning these significant differences, the disclosure provides an analysis of the association between anti-PD-1 efficacy, MT-HGs, and tumor PD-L1 status, as both NIVO and PEMBRO disrupt the PD-1/PD-L1 axis. Although PD-L1 status is not routinely captured in SOC ICI treatment of MM patients, it was recorded for all patients on the CheckMate-067 trial. We therefore performed a sub-analysis, stratified by MT-HG status, for the association between anti-PD-1-based treatment efficacy and PD-L1 status for NIVO- and COMBO-treated patients on CheckMate-067. While the majority of PD-L1+ patients with other haplogroups tended to experience NIVO CB (60 of 99 (61%) PD-L1+ NIVO CB), ALL HG-T NIVO CB patients were PD-L1+ (6 of 6 (100%) PD-L1+ NIVO CB) (Table 13). For patients treated with COMBO, the association with PD-L1 positivity was not as strong for the other MT-HGs (50 of 109 (46%) PD-L1+ COMBO CB), but remained high for HG-T (2 of 3 (67%) PD-L1+ COMBO CB) (Table 13). Combining the NIVO and COMBO cohorts (Table 2-6b), we observed a statistically significant enrichment of PD-L1 positivity among HG-T CB patients (8 of 9 (89%) PD-L1+ CB) compared to CB patients with other MT-HGs (110 of 208 (53%) PD-L1+ CB) (chi-square p-value=0.03).

These findings, from nearly 1,500 patients from a clinical trial and large international SOC consortium, collectively provide the first evidence of differences in the efficacy of two different anti-PD-1 mAbs based on mitochondrial genetic makeup. Specifically, HG-T patients are significantly more likely to experience CB when treated with PEMBRO as compared to IPI, NIVO, or COMBO. This is in contrast to patients with other MT-HGs, who are most likely to experience CB from COMBO. In CheckMate-067, we also observed significant enrichment of PD-L1 positivity among HG-T patients who experienced CB from NIVO-based ICI. Whereas approximately half of CB patients with other MT-HGs were PD-L1+, we observed almost 90% PD-L1 positivity in HG-T CB patients. These results strongly indicate a need for a parallel assessment of MT-HG status and PD-L1 expression in determination of a more personalized selection of anti-PD-1 therapy option with the improved expected treatment outcome.

### Discussion of the Foregoing Examples

Without intending to be bound by any particular theory, it is considered that this disclosure provides for the first time, testing and validation of the association of inherited mitochondrial haplogroups (MT-HGs) with ICI efficacy in melanoma, providing a novel link between mitochondrial inheritance and ICI-mediated anti-tumor immune response.

Analyzing the mitochondrial genome of 1,254 ICI-treated metastatic melanoma (MM) patients, we found that ICI efficacy varies by MT-HGs. We discovered that mitochondrial haplogroup T (HG-T) is significantly associated with resistance to NIVO (pooled OR=3.53, p-value=2.22E-04), which we validated in both a clinical trial (CheckMate-067) as well as in an independent standard-of-care ICI cohort from an international consortium (IO-GEM). This is the first inherited genetic marker predicting patients before treatment who will not benefit from single-agent NIVO, a treatment option with otherwise high efficacy and low toxicity.⁶⁵ In alignment with these observations, NIVO-resistant HG-T patients show poor PFS as well as OS, when compared to other MT-HGs treated by single-agent NIVO.

This disclosure also shows that HG-T patients experience comparably poor outcomes with anti-PD-1/anti-CTLA-4 combination therapy (COMBO) (31% ORR, OR=3.73, p=4.43E-04), which is also a frontline ICI therapy in MM. With no statistical difference observed in anti-CTLA-4 efficacy between HG-T and other MT-HGs, these findings collectively indicate that HG-T is specifically associated with resistance to NIVO-based treatments (single-agent or in combination with anti-CTLA-4), and this also correlates with significantly poorer survival. The disclosure thus supports use of other ICI treatment combinations that HG-T patients may benefit from, particularly in light of recent development of new combination ICIs that include blockade of other immune checkpoints (e.g. LAG-3, TIGIT, TIM-3, etc.⁵⁵) or alternative immunotherapies such as recently approved adoptive cell therapies in melanoma.⁷²

This disclosure reveals a potential link between mitochondrial inheritance, host immune cell repertoire, and ICI efficacy. It has been proposed that PD-1 inhibition returns the cytotoxic capacities of certain phenotypes of exhausted CD8+ T cells, possibly improving anti-tumor immunity. In contrast, dysfunctional cells (terminally exhausted) can no longer be reinvigorated by PD-1 inhibition.^{58,59,62} Our scRNA-seq data show that the peripheral baseline distribution of these exhausted cell phenotypes varies by MT-HG and treatment efficacy in NIVO-treated patients. We have identified three distinct peripheral CD8⁺ effector T cell exhaustion phenotypes by NIVO response and MT-HG status. We observed that CB patients exhibit higher baseline levels of cell phenotypes capable of regaining cytotoxic activity by NIVO reinvigoration. In contrast, NCB patients of non-T MT-HGs show a lesser fraction of these cells, but more dysfunctional phenotypes, no longer able to be reinvigorated by NIVO. NCB patients with HG-T show significantly lower overall baseline levels of all differentiated CD8+ T-cell states, neither dysfunctional nor those that can be PD-1 reinvigorated. This NIVO-resistance-associated CD8+ T cell phenotype possibly points to an altered differentiation cascade, which, in HG-T patients, retains CD8+ T cells at earlier differentiation stages.

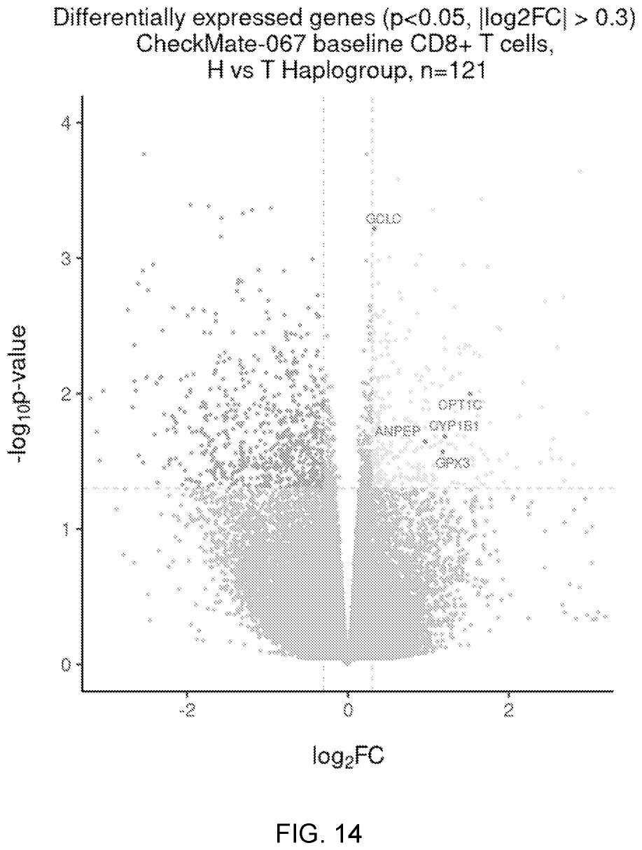

These data indicate that MT-HG status may be associated with the processes that drive CD8⁺ cells into effector states and this capacity affects response to ICI. While the biological mechanisms explaining these correlations are elusive, they are consistent with recent studies showing that T cell differentiation to effector states depends on differential tolerance to reactive oxygen species (ROS),²³ which varies by MT-HGs. HG-T can better tolerate ROS damage compared to other MT-HGs.¹⁸ Upon PD-1 blockade, ROS also stimulate the AMPK/mTOR pathway, leading to an increase in TBX21 production and a decrease in EOMES.²¹ Based on this evidence, in individuals with greater tolerance to ROS damage, such as those with HG-T, more ROS may be needed to drive the cells into the activated state. While not intending to be constrained by any particular interpretation, this may explain why we observed very few effector cells in the CD8⁺ repertoire of HG-T patients not responding to NIVO. The described results linking mitochondrial haplogroups and response to ICI support the involvement of mitochondria-associated ROS in T cell differentiation. The transcriptional profiles of CD8+ T cells from HG-T NIVO-resistant patients from CheckMate-067 are enriched by gene signatures in pathways related to ROS detoxification, including GCLC, CPTIC, ANPEP, CYP1B1, and GPX3 (FIG. 14). This finding provides support for the hypothesis that the baseline CD8+ T cell phenotype in NIVO-resistant HG-T patients may be associated with their ability to resist ROS damage.

We additionally found that patients with mitochondrial haplogroup T (HG-T) are significantly more likely to respond to pembrolizumab (PEMBRO) compared to nivolumab (NIVO), with over 5 times the odds of no clinical benefit (NCB) in HG-T patients treated with NIVO compared to PEMBRO (HG-T NCB OR for NIVO vs PEMBRO: 5.43 (95% CI: 1.87-16.9), p-value=2.40E-03), both ICIs targeting the PD-1 receptor. The significant difference in PEMBRO outcomes for HG-T was also present when assessing outcomes in the NIVO-based combination treatment, which again showed significantly poorer efficacy (HG-T NCB OR for COMBO vs PEMBRO: 3.62 (95%: 1.19-11.8), p-value-0.03), providing more evidence that the disclosure reveals a NIVO-based resistance marker. That other haplogroups follow the expected response distribution, with virtually identical outcomes when comparing NIVO and PEMBRO (Other MT-HG NCB OR for NIVO vs. PEMBRO: 1.01 (95% CI: 0.72-1.41), p-value=0.98), possibly explains why there was little difference observed between the two therapies in the aggregate ICI analyses.^{67,73} It is also possible, therefore, that differences in the underlying haplogroup distributions may account for some of the observed variation in outcomes when the two treatments are compared in parallel. Taken together, these data provide evidence that, for a subset of patients, these two drugs should be administered rationally. These findings also clearly point to possible differences in the mechanism of action for each of these mAbs when inhibiting the PD-1/PD-L1 axis. Although they target the same receptor, NIVO and PEMBRO are not structurally identical antibodies, and these structural variations may affect how they interact with the corresponding PD-L1 ligand.⁷⁴ The primary difference is in the variable region of the two antibodies, which generates the antibody's paratope, the region of the antibody that binds to the antigen epitope.⁶⁹ Specifically, the PEMRBO epitope region covers a much greater extent of the PD-L1 binding site of the PD-1 receptor compared to the NIVO epitope region.⁶⁹ Despite their other similarities, there is almost no overlap in the PD-1 molecule binding sites between NIVO and PEMBRO, including where they overlap with the PD-1/PD-L1 binding site.⁶⁹⁻⁷¹ Further, recent evidence shows that PD-1 is capable of dimerization in cis (on the same T cell surface), and this propensity for dimerization may be associated with decreased cytotoxic T cell function.⁷⁵

Given these structural differences between the two anti-PD1 mAbs in relation to the PD-L1 binding site, the present disclosure supports the interpretation that even HG-T patients with lower tumor PD-L1 expression may benefit from PEMBRO treatment, and it can be expected that this improvement to be even more pronounced for patients with high PD-L1 expression. This is supported by the present data, showing that PEMBRO provides superior clinical benefit in HG-T patients, who are otherwise largely resistant to NIVO. All patients with HG-T that responded to NIVO are PD-L1 positive, further supporting a model that PD-L1 positivity may be an important factor in determining anti-PD-1 clinical benefit for HG-T patients. Of nine total HG-T patients experiencing CB from NIVO-based treatments on CheckMate-067, eight were PD-L1 positive. The link between PD-L1 status and HG-T NIVO-resistance also remained when we assessed melanoma patients treated with adjuvant NIVO or COMBO therapy on the CheckMate-915 clinical trial (data not shown). When evaluating only the patients with disease recurrence (the clearest measure of treatment efficacy in the adjuvant setting wherein some patients are cured by surgery alone), 12 of 13 HG-T patients who recurred were PD-L1 negative, including all single-agent NIVO-treated HG-T patients.

This association between efficacy and PD-L1 status is consistent with our bulk and single-cell (sc) RNA-seq data that lead us to hypothesize that the overall lower proportion of differentiated effector T cells in NIVO-resistant HG-T patients may be related to their ability to resist reactive oxygen species (ROS). ROS have been shown to induce tumor PD-L1 expression⁷⁶⁻⁷⁸ while ROS scavenging tends to repress PD-L1.76 The one HG-T patient with scRNA-seq data who responded to NIVO showed a much more differentiated baseline immune portfolio and was PD-L1 positive, while five of the six that were NIVO-resistant were PD-L1 negative. It is therefore possible that, for HG-T patients, there is a synergistic relationship between their ability to resist ROS, the extent of differentiation in their immune repertoire, and their level of tumor PD-L1 positivity.

The present disclosure includes the large sample size (n=1,254 ICI-treated patients), with a discovery and validation dataset generated from well-controlled clinical trial samples (CheckMate-067) and a standard-of-care treatment cohort from a multinational study (IO-GEM), ensuring the rigor and reproducibility of the findings. Combining these cohorts generated the largest genomic analysis to date of ICI-treated MM patients, and the first to explore the role of inherited mitochondrial genetics in ICI efficacy. Further, unlike most previous studies that combine patients into one “ICI-treated” population, in the present disclosure, stratifying by treatment allowed analysis of outcomes separately by ICI therapy as well as by haplogroup. The addition of scRNA-seq data is an aspect of the disclosure, extending the observational findings to identify a relevant peripheral host cell phenotype connecting MT-HGs with a putative biological model of HG-T impacting ROS resistance and ICI efficacy.

As discussed above, it is believed this disclosure provides the first evidence that the MT-HG status is associated with ICI outcomes. The disclosure supports the use of MT-HG genetic assessment for use as a clinically viable, minimally invasive baseline biomarker that can identify patients who are less likely to respond to current NIVO-based treatment regimens. In an aspect, it is considered the disclosure reveals for the first time the connection between mitochondrial genetics and the peripheral blood immune cell repertoire in the context of ICI resistance.

The results demonstrate that the T haplogroup correlates with poor response to current NIVO-based therapy and indicate that other haplogroups also respond differently relative to the expected overall response. Given the present description of better response in T haplogroup patients receiving PEMBRO, the disclosure includes analysis of outcomes of T haplogroup patients treated with novel anti-PD-1-based treatments such as those combining NIVO with anti-LAG-3 therapy. As all currently FDA-approved predictive biomarkers (PD-L1⁷⁹ and MSI status,⁸⁰ TMB⁸¹) require tumor samples, mitochondrial haplogrouping represents an approach for a novel, conveniently obtainable baseline predictor of subsequent treatment response. Thus, the disclosure supports the use of mitochondrial haplogrouping of patients as a clinically viable, minimally invasive baseline biomarker that can identify patients who are less likely to respond to current NIVO-based treatment regimens and that these patients would respond better to PEMBRO treatment. Given the ease of germline genetic testing along with the clear evidence of this disclosure demonstrating heterogeneous responses to mainstream ICI therapies across haplogroups, the disclosure includes analyzing historic ICI combinations that have been discarded due to their worse/unaltered response rates to test if they are actually benefiting patients with specific haplogroups. Accordingly, these blood tests may maximize the utility of existing immunotherapies while improving response to treatment by creating new personalized treatment regimens.

This reference listing is not an indication that any reference is material to patentability.
1 Larkin, J. et al. Combined Nivolumab and Ipilimumab or Monotherapy in Untreated Melanoma. New Engl J Med 373, 23-34 (2015). https://doi.org: 10.1056/NEJMoa1504030
2 Hodi, F. S. et al. Nivolumab plus ipilimumab or nivolumab alone versus ipilimumab alone in 2 advanced melanoma (CheckMate 067): 4-year outcomes of a multicentre, randomised, phase 3 trial. The Lancet Oncology 19, 1480-1492 (2018). https://doi.org: 10.1016/S1470-2045 (18) 30700-9
3 Larkin, J. et al. Five-Year Survival with Combined Nivolumab and Ipilimumab in Advanced Melanoma. N Engl J Med 381, 1535-1546 (2019). https://doi.org: 10.1056/NEJMoa1910836
4 Albertini, M. R. The age of enlightenment in melanoma immunotherapy. Journal for ImmunoTherapy of Cancer 6, 80 (2018). https://doi.org: 10.1186/s40425-018-0397-8
5 Hofmann, L. et al. Cutaneous, gastrointestinal, hepatic, endocrine, and renal side-effects of anti-PD-1 therapy. Eur J Cancer 60, 190-209 (2016). https://doi.org: 10.1016/j.ejca.2016.02.025
6 Postow, M. A. et al. Nivolumab and ipilimumab versus ipilimumab in untreated melanoma. N Engl J Med 372, 2006-2017 (2015). https://doi.org: 10.1056/NEJMoa1414428
7 Yang, Y. et al. Comparative Efficacy and Safety of Nivolumab and Nivolumab Plus Ipilimumab in Advanced Cancer: A Systematic Review and Meta-Analysis. Frontiers in pharmacology 11, 40 (2020). https://doi.org: 10.3389/fphar.2020.00040
8 Johnson, D. B. et al. Targeted Next Generation Sequencing Identifies Markers of Response to PD-1 Blockade. Cancer Immunol Res 4, 959-967 (2016). https://doi.org: 10.1158/2326-6066.Cir-16-0143
9 Snyder, A. et al. Genetic basis for clinical response to CTLA-4 blockade in melanoma. N Engl J Med 371, 2189-2199 (2014). https://doi.org: 10.1056/NEJMoa1406498
10 Rizvi, N. A. et al. Cancer immunology. Mutational landscape determines sensitivity to PD-1 blockade in non-small cell lung cancer. Science 348, 124-128 (2015). https://doi.org: 10.1126/science.aaa1348
11 Tumeh, P. C. et al. PD-1 blockade induces responses by inhibiting adaptive immune resistance. Nature 515, 568-+ (2014). https://doi.org: 10.1038/nature 13954
12 Herbst, R. S. et al. Predictive correlates of response to the anti-PD-L1 antibody MPDL3280A in cancer patients. Nature 515, 563-567 (2014). https://doi.org: 10.1038/nature 14011
13 Spranger, S. et al. Up-Regulation of PD-L1, IDO, and T-regs in the Melanoma Tumor Microenvironment Is Driven by CD8 (+) T Cells. Sci Transl Med 5 (2013). https://doi.org: 10.1126/scitranslmed.3006504
14 Spain, L. et al. Late-stage metastatic melanoma emerges through a diversity of evolutionary pathways. Cancer discovery (2023). https://doi.org: 10.1158/2159-8290.CD-22-1427
15 Sena, L. A. et al. Mitochondria are required for antigen-specific T cell activation through reactive oxygen species signaling. Immunity 38, 225-236 (2013). https://doi.org: 10.1016/j.immuni.2012.10.020
16 van der Windt, G. J. et al. CD8 memory T cells have a bioenergetic advantage that underlies their rapid recall ability. Proceedings of the National Academy of Sciences of the United States of America 110, 14336-14341 (2013). https://doi.org: 10.1073/pnas. 1221740110
17 Patel, T. H. et al. European mtDNA Variants Are Associated With Differential Responses to Cisplatin, an Anticancer Drug: Implications for Drug Resistance and Side Effects. Front Oncol 9, 640 (2019). https://doi.org: 10.3389/fonc.2019.00640
18 Mueller, E. E. et al. Functional differences between mitochondrial haplogroup T and haplogroup H in HEK293 cybrid cells. PLOS One 7, e52367 (2012). https://doi.org: 10.1371/journal.pone.0052367
19 Panvini, A. R. et al. Differential mitochondrial and cellular responses between H vs. J mtDNA haplogroup-containing human RPE transmitochondrial cybrid cells. Exp Eye Res 219, 109013 (2022). https://doi.org: 10.1016/j.exer.2022.109013
20 Li, C. et al. A high OXPHOS CD8 T cell subset is predictive of immunotherapy resistance in melanoma patients. The Journal of experimental medicine 219 (2022). https://doi.org: 10.1084/jem.20202084
21 Chamoto, K. et al. Mitochondrial activation chemicals synergize with surface receptor PD-1 blockade for T cell-dependent antitumor activity. Proceedings of the National Academy of Sciences of the United States of America 114, E761-E770 (2017). https://doi.org: 10.1073/pnas. 1620433114
22 Kumar, A., Chamoto, K., Chowdhury, P. S. & Honjo, T. Tumors attenuating the mitochondrial activity in T cells escape from PD-1 blockade therapy. Elife 9 (2020). https://doi.org: 10.7554/eLife.52330
23 Chowdhury, P. S., Chamoto, K., Kumar, A. & Honjo, T. PPAR-Induced Fatty Acid Oxidation in T Cells Increases the Number of Tumor-Reactive CD8 (+) T Cells and Facilitates Anti-PD-1 Therapy. Cancer Immunol Res 6, 1375-1387 (2018). https://doi.org: 10.1158/2326-6066.CIR-18-0095
24 Eisenhauer, E. A. et al. New response evaluation criteria in solid tumours: revised RECIST guideline (version 1.1). Eur J Cancer 45, 228-247 (2009). https://doi.org: 10.1016/j.ejca.2008.10.026
25 Delgado, A. & Guddati, A. K. Clinical endpoints in oncology—a primer. American journal of cancer research 11, 1121-1131 (2021).
26 Liu, D. et al. Integrative molecular and clinical modeling of clinical outcomes to PD1 blockade in patients with metastatic melanoma. Nat Med 25, 1916-1927 (2019). https://doi.org: 10.1038/s41591-019-0654-5
27 Hugo, W. et al. Genomic and Transcriptomic Features of Response to Anti-PD-1 Therapy in Metastatic Melanoma. Cell 165, 35-44 (2016). https://doi.org: 10.1016/j.cell.2016.02.065
28 Chat, V. et al. A genome-wide association study of germline variation and melanoma 28 prognosis. Frontiers in Oncology 12 (2023). https://doi.org: 10.3389/fonc.2022.1050741
29 Wich, L. G. et al. Developing a multidisciplinary prospective melanoma biospecimen repository to advance translational research. Am J Transl Res 1, 35-43 (2009).
30 Vogelsang, M. et al. The Expression Quantitative Trait Loci in Immune Pathways and their Effect on Cutaneous Melanoma Prognosis. Clin Cancer Res 22, 3268-3280 (2016). https://doi.org: 10.1158/1078-0432.Ccr-15-2066
31 Auton, A. et al. A global reference for human genetic variation. Nature 526, 68-74 (2015). https://doi.org: 10.1038/nature15393
32 Van der Auwera, G. A. & O'Connor, B. D. Genomics in the Cloud: Using Docker, GATK, and WDL in Terra. Vol. 1 (O'Reilly Media, 2020).
33 Li, H. & Durbin, R. Fast and accurate long-read alignment with Burrows-Wheeler transform. Bioinformatics (Oxford, England) 26, 589-595 (2010). https://doi.org: 10.1093/bioinformatics/btp698
34 Tarasov, A., Vilella, A. J., Cuppen, E., Nijman, I. J. & Prins, P. Sambamba: fast processing of NGS alignment formats. Bioinformatics (Oxford, England) 31, 2032-2034 (2015). https://doi.org: 10.1093/bioinformatics/btv098% J Bioinformatics
35 Danecek, P. et al. Twelve years of SAMtools and BCFtools. GigaScience 10 (2021). https://doi.org: 10.1093/gigascience/giab008
36 Schönherr, S., Weissensteiner, H., Kronenberg, F. & Forer, L. Haplogrep 3-an interactive haplogroup classification and analysis platform. Nucleic Acids Research (2023). https://doi.org: 10.1093/nar/gkad284
37 Dür, A., Huber, N. & Parson, W. Fine-Tuning Phylogenetic Alignment and Haplogrouping of mtDNA Sequences. International journal of molecular sciences 22 (2021). https://doi.org: 10.3390/ijms22115747
38 Zheng, G. X. Y. et al. Massively parallel digital transcriptional profiling of single cells. Nature Communications 8, 14049 (2017). https://doi.org: 10.1038/ncomms14049
39 Butler, A., Hoffman, P., Smibert, P., Papalexi, E. & Satija, R. Integrating single-cell transcriptomic data across different conditions, technologies, and species. Nature biotechnology 36, 411-420 (2018). https://doi.org: 10.1038/nbt.4096
40 Satija, R., Farrell, J. A., Gennert, D., Schier, A. F. & Regev, A. Spatial reconstruction of single-cell gene expression data. Nature biotechnology 33, 495-502 (2015). https://doi.org: 10.1038/nbt.3192
41 Stuart, T. et al. Comprehensive Integration of Single-Cell Data. Cell 177, 1888-1902.e1821 (2019). https://doi.org: https://doi.org/10.1016/j.cell.2019.05.031
42 Hao, Y. et al. Integrated analysis of multimodal single-cell data. Cell 184, 3573-3587.e3529 (2021). https://doi.org: https://doi.org/10.1016/j.cell.2021.04.048
43 Chat, V. et al. Autoimmune genetic risk variants as germline biomarkers of response to melanoma immune-checkpoint inhibition. Cancer immunology, immunotherapy: CII 68, 897-905 (2019). https://doi.org: 10.1007/s00262-019-02318-8
44 Purcell, S. et al. PLINK: a tool set for whole-genome association and population-based linkage analyses. Am J Hum Genet 81, 559-575 (2007). https://doi.org: 10.1086/519795
45 Purcell, S. PLINK 1.9/2.0, <http://pngu.mgh.harvard.edu/purcell/plink/> (46
46 Chat, V., Ferguson, R., Morales, L. & Kirchhoff, T. Ultra Low-Coverage Whole-Genome Sequencing as an Alternative to Genotyping Arrays in Genome-Wide Association Studies. Frontiers in genetics 12, 790445 (2021). https://doi.org: 10.3389/fgene.2021.790445
47 Diroma, M. A. et al. Extraction and annotation of human mitochondrial genomes from 1000 Genomes Whole Exome Sequencing data. BMC Genomics 15, S2 (2014). https://doi.org: 10.1186/1471-2164-15-S3-S2
48 Griffin, H. R. et al. Accurate mitochondrial DNA sequencing using off-target reads provides a single test to identify pathogenic point mutations. Genetics in Medicine 16, 962-971 (2014). https://doi.org: 10.1038/gim.2014.66
49 Zhang, Y. et al. High copy number of mitochondrial DNA (mtDNA) predicts good prognosis in glioma patients. American journal of cancer research 5, 1207-1216 (2015).
50 Hodi, F. S. et al. TMB and Inflammatory Gene Expression Associated with Clinical Outcomes following Immunotherapy in Advanced Melanoma. Cancer Immunology Research 9, 1202-1213 (2021). https://doi.org: 10.1158/2326-6066.Cir-20-0983
51 Paradis, E. & Schliep, K. ape 5.0: an environment for modern phylogenetics and evolutionary analyses in R. Bioinformatics (Oxford, England) 35, 526-528 (2018). https://doi.org: 10.1093/bioinformatics/bty633% J Bioinformatics
52 gt: Easily Create Presentation-Ready Display Tables (2024).
53 A Package for Survival Analysis in R (2023).
54 Mallilankaraman, K. B. in Immunity and Inflammation in Health and Disease (eds Shampa Chatterjee, Wolfgang Jungraithmayr, & Debasis Bagchi) 57-64 (Academic Press, 2018).
55 Cai, L., Li, Y., Tan, J., Xu, L. & Li, Y. Targeting LAG-3, TIM-3, and TIGIT for cancer immunotherapy. Journal of Hematology & Oncology 16, 101 (2023). https://doi.org: 10.1186/s13045-023-01499-1
56 Nolz, J. C. Molecular mechanisms of CD8 (+) T cell trafficking and localization. Cell Mol Life Sci 72, 2461-2473 (2015). https://doi.org: 10.1007/s00018-015-1835-0
57 Giles, J. R., Globig, A. M., Kaech, S. M. & Wherry, E. J. CD8 (+) T cells in the cancer-immunity cycle. Immunity 56, 2231-2253 (2023). https://doi.org: 10.1016/j.immuni.2023.09.005
58 Wherry, E. J. T cell exhaustion. Nature Immunology 12, 492-499 (2011). https://doi.org: 10.1038/ni.2035
59 Berrien-Elliott, M. M. et al. Checkpoint blockade immunotherapy relies on T-bet but not Eomes to induce effector function in tumor-infiltrating CD8+ T cells. Cancer Immunol Res 3, 116-124 (2015). https://doi.org: 10.1158/2326-6066.Cir-14-0159
60 Schietinger, A. & Greenberg, P. D. Tolerance and exhaustion: defining mechanisms of T cell dysfunction. Trends in immunology 35, 51-60 (2014). https://doi.org: 10.1016/j.it.2013.10.001
61 Paley, M. A. et al. Progenitor and terminal subsets of CD8+ T cells cooperate to contain chronic viral infection. Science 338, 1220-1225 (2012). https://doi.org: 10.1126/science.1229620
62 Wherry, E. J. & Kurachi, M. Molecular and cellular insights into T cell exhaustion. Nature reviews. Immunology 15, 486-499 (2015). https://doi.org: 10.1038/nri3862
63 Blackburn, S. D., Shin, H., Freeman, G. J. & Wherry, E. J. Selective expansion of a subset of exhausted CD8 T cells by alphaPD-L1 blockade. Proceedings of the National Academy of Sciences of the United States of America 105, 15016-15021 (2008). https://doi.org: 10.1073/pnas.0801497105
64 Khan, O. et al. TOX transcriptionally and epigenetically programs CD8+ T cell exhaustion. Nature 571, 211-218 (2019). https://doi.org: 10.1038/s41586-019-1325-x
65 Buchbinder, E. I. & Desai, A. CTLA-4 and PD-1 Pathways: Similarities, Differences, and Implications of Their Inhibition. American Journal of Clinical Oncology 39, 98-106 (2016). https://doi.org: 10.1097/coc.0000000000000239
66 Moser, J. C. et al. Comparative-effectiveness of pembrolizumab vs. nivolumab for patients with metastatic melanoma. Acta Oncologica 59, 434-437 (2020). https://doi.org: 10.1080/0284186X.2020.1712473
67 Cybulska-Stopa, B. et al. Long-term clinical evidence of comparable efficacy and toxicity of nivolumab and pembrolizumab in advanced melanoma treatment. Melanoma Research 33, 208-217 (2023). https://doi.org: 10.1097/cmr.0000000000000885
68 Cui, P. et al. Comparative effectiveness of pembrolizumab vs. nivolumab in patients with recurrent or advanced NSCLC. Scientific Reports 10, 13160 (2020). https://doi.org: 10.1038/s41598-020-70207-7
69 Fessas, P., Lee, H., Ikemizu, S. & Janowitz, T. A molecular and preclinical comparison of the PD-1-targeted T-cell checkpoint inhibitors nivolumab and pembrolizumab. Seminars in Oncology 44, 136-140 (2017). https://doi.org: https://doi.org/10.1053/j.seminoncol.2017.06.002
70 Tan, S. et al. An unexpected N-terminal loop in PD-1 dominates binding by nivolumab. Nature Communications 8, 14369 (2017). https://doi.org: 10.1038/ncomms14369
71 Horita, S. et al. High-resolution crystal structure of the therapeutic antibody pembrolizumab bound to the human PD-1. Scientific Reports 6, 35297 (2016). https://doi.org: 10.1038/srep35297
72 Chesney, J. et al. Efficacy and safety of lifileucel, a one-time autologous tumor-infiltrating lymphocyte (TIL) cell therapy, in patients with advanced melanoma after progression on immune checkpoint inhibitors and targeted therapies: pooled analysis of consecutive cohorts of the C-144-01 study. J Immunother Cancer 10 (2022). https://doi.org: 10.1136/jitc-2022-005755
73 Moser, J. C. et al. Comparative-effectiveness of pembrolizumab vs. nivolumab for patients with metastatic melanoma. Acta Oncol 59, 434-437 (2020). https://doi.org: 10.1080/0284186x.2020.1712473
74 Mittal, L. et al. Interplay among Structural Stability, Plasticity, and Energetics Determined by Conformational Attuning of Flexible Loops in PD-1. Journal of Chemical Information and Modeling 61, 358-384 (2021). https://doi.org: 10.1021/acs.jcim.0c01080
75 Philips, E. A. et al. Transmembrane domain-driven PD-1 dimers mediate T cell inhibition. Science Immunology 9, eade6256 (2024). https://doi.org: doi: 10.1126/sciimmunol.ade6256
76 Bailly, C. Regulation of PD-L1 expression on cancer cells with ROS-modulating drugs. Life Sci 246, 117403 (2020). https://doi.org: 10.1016/j.lfs.2020.117403
77 Kou, Z. et al. Oxidative stress modulates expression of immune checkpoint genes via activation of AhR signaling. Toxicology and Applied Pharmacology 457, 116314 (2022). https://doi.org: https://doi.org/10.1016/j.taap.2022.116314
78 Kuo, C.-L. et al. Mitochondrial oxidative stress in the tumor microenvironment and cancer immunoescape: foe or friend? Journal of Biomedical Science 29, 74 (2022). https://doi.org: 10.1186/s12929-022-00859-2
79 Davis, A. A. & Patel, V. G. The role of PD-L1 expression as a predictive biomarker: an analysis of all US Food and Drug Administration (FDA) approvals of immune checkpoint inhibitors. J Immunother Cancer 7, 278 (2019). https://doi.org: 10.1186/s40425-019-0768-9
80 Boyiadzis, M. M. et al. Significance and implications of FDA approval of pembrolizumab for biomarker-defined disease. J Immunother Cancer 6, 35 (2018). https://doi.org: 10.1186/s40425-018-0342-x
81 Sha, D. et al. Tumor Mutational Burden as a Predictive Biomarker in Solid Tumors. Cancer discovery 10, 1808-1825 (2020). https://doi.org: 10.1158/2159-8290.Cd-20-0522

TABLE 1
Results of CD8+ T-cell differential expression analysis of:
A. Discovery Cohort of patients treated by single-agent Nivolumab ICI (n = 70) (Complete Response (CR),
n = 14; Partial Response (PR), n = 15; Stable Disease (SD), n-6 Progressive Disease (PD), n = 35).
B. Validation Cohort of patients treated by single-agent Nivolumab ICI
(n = 46) (CR, n = 7; PR, n = 12; SD, n = 3; PD, n = 24).
C. Meta-Analysis (Discovery and Validation Cohorts) of patients treated by single-agent
Nivolumab ICI (n = 116) (CR, n = 21; PR, n = 27; SD, n = 9; PD, n = 59).
The expression is derived from RNA-seq analysis of baseline (pre-treatment) peripheral
blood CD8+ T-cells. The table lists the top 10 genes that are most significantly
differentially expressed (p < 0.01, |log₂FC| > 1) in each response comparison.

| | | | | | | |
|---|---|---|---|---|---|---|
| Rank | Gene | log₂FoldChange | p-value | FDR adj. p | gene biotype | description |

A. NIVO RNA-seq Discovery Cohort, n = 70
CB (CR, n = 14; PR, n = 15) vs NCB (SD, n = 6; PD, n = 35)

| | | | | | | |
|---|---|---|---|---|---|---|
| 1 | HMAX | 4.124786034 | 4.37E−13 | 8.87E−09 | protein coding | HMAX |
| 2 | HLX | 2.247898968 | 8.60E−07 | 0.008730808 | protein coding | H2.0 like homeobox |
| 3 | FFAR2 | 3.006203835 | 1.88E−06 | 0.012717011 | protein coding | free fatty acid receptor 2 |
| 4 | LYZ | 1.702383589 | 4.54E−06 | 0.015365051 | protein coding | lysozyme |
| 5 | SERPINA1 | 1.467126048 | 4.20E−06 | 0.015365051 | protein coding | serpin family A member 1 |
| 6 | C5AR1 | 1.917016154 | 3.47E−06 | 0.015365051 | protein coding | complement C5a receptor 1 |
| 7 | HOXA7 | −2.034423079 | 6.09E−06 | 0.017651538 | protein coding | homeobox A7 |
| 8 | SLC11A1 | 1.598853412 | 2.82E−05 | 0.063883114 | protein coding | solute carrier family 11 member 1 |
| 9 | FCN1 | 1.729496242 | 3.64E−05 | 0.063883114 | protein coding | ficolin 1 |
| 10 | WTIP | −1.431375059 | 3.94E−05 | 0.063883114 | protein coding | WT1 interacting protein |

CB (CR, n = 14; PR, n = 15) vs NCB (PD, n = 35)

| | | | | | | |
|---|---|---|---|---|---|---|
| 1 | HMAX | 4.313132797 | 2.03E−13 | 4.17E−09 | protein coding | HMAX |
| 2 | LYZ | 1.778638509 | 1.44E−06 | 0.011205081 | protein coding | lysozyme |
| 3 | C5AR1 | 2.018301272 | 1.64E−06 | 0.011205081 | protein coding | complement C5a receptor 1 |
| 4 | SERPINA1 | 1.401995364 | 5.23E−06 | 0.026819112 | protein coding | serpin family A member 1 |
| 5 | HOXA7 | −2.058985011 | 1.22E−05 | 0.049895199 | protein coding | homeobox A7 |
| 6 | FCN1 | 1.811497847 | 2.03E−05 | 0.054323819 | protein coding | ficolin 1 |
| 7 | FFAR2 | 2.675487121 | 1.63E−05 | 0.054323819 | protein coding | free fatty acid receptor 2 |
| 8 | WTIP | −1.506273733 | 2.76E−05 | 0.054323819 | protein coding | WT1 interacting protein |
| 9 | CLCN5 | 1.527630214 | 2.91E−05 | 0.054323819 | protein coding | chloride voltage-gated channel 5 |
| 10 | SLC11A1 | 1.634678133 | 4.06E−05 | 0.067247951 | protein coding | solute carrier family 11 member 1 |

B. NIVO RNA-seq Validation, n = 46
CB (CR, n = 7; PR, n = 12) vs. NCB (SD, n = 3; PD, n = 24)

| | | | | | | |
|---|---|---|---|---|---|---|
| 1 | HMAX | 5.131617118 | 7.70E−11 | 1.63E−06 | protein coding | MT-RNR2 like 8 |
| 2 | WDFY4 | 3.255290925 | 2.74E−09 | 2.90E−05 | protein coding | WDFY family member 4 |
| 3 | GUCY1A1 | 2.604583926 | 7.97E−07 | 0.005618073 | protein coding | Guanylate cyclase soluble subunit |
| | | | | | | alpha-1 |
| 4 | CD79A | 1.653473616 | 1.65E−05 | 0.070383653 | protein coding | Cluster of differentiation CD79A |
| 5 | NIBAN3 | 2.33059601 | 1.66E−05 | 0.070383653 | protein coding | niban apoptosis regulator 3 |
| 6 | ADAM28 | 1.889793852 | 3.15E−05 | 0.072088372 | protein coding | ADAM metallopeptidase domain |
| | | | | | | 28 |
| 7 | COL19A1 | 2.464579374 | 3.31E−05 | 0.072088372 | protein coding | collagen type XIX alpha 1 chain |
| 8 | PTPRF | 3.355842884 | 2.75E−05 | 0.072088372 | protein coding | protein tyrosine phosphatase |
| | | | | | | receptor type F |
| 9 | TSPAN33 | 1.775605012 | 3.41E−05 | 0.072088372 | protein coding | tetraspanin 33 |
| 10 | PROS1 | 3.366300942 | 3.16E−05 | 0.072088372 | protein coding | protein S |

CB (CR, n = 7; PR, n = 12) vs. NCB (PD, n = 24)

| | | | | | | |
|---|---|---|---|---|---|---|
| 1 | WDFY4 | 2.560772356 | 3.14E−08 | 0.000656156 | protein coding | WDFY family member 4 |
| 2 | CD22 | 2.811976558 | 6.62E−07 | 0.004621567 | protein coding | CD22 molecule |
| 3 | HMAX | 4.12399036 | 5.59E−07 | 0.004621567 | protein coding | HMAX |
| 4 | GUCY1A1 | 2.623291772 | 1.48E−06 | 0.007735769 | protein coding | guanylate cyclase 1 soluble |
| | | | | | | subunit alpha 1 |
| 5 | PTPRF | 3.538340111 | 7.99E−06 | 0.033463773 | protein coding | protein tyrosine phosphatase |
| | | | | | | receptor type F |
| 6 | NIBAN3 | 2.403339229 | 1.12E−05 | 0.03918731 | protein coding | niban apoptosis regulator 3 |
| 7 | CD79A | 1.547500575 | 2.01E−05 | 0.060148593 | protein coding | Cluster of differentiation CD79A |
| 8 | COL19A1 | 2.497925557 | 4.78E−05 | 0.118368878 | protein coding | collagen type XIX alpha 1 chain |
| 9 | HOMER2 | 2.163677712 | 5.09E−05 | 0.118368878 | protein coding | homer scaffold protein 2 |
| 10 | MAP1B | 1.983631641 | 0.000123201 | 0.161161872 | protein coding | microtubule associated protein 1B |

C. NIVO RNA-seq Meta-Analysis, n = 116
CB (CR, n = 21; PR, n = 27) vs. NCB (SD, n = 9; PD, n = 59)

| | | | | | | |
|---|---|---|---|---|---|---|
| 1 | HMAX | 4.033424762 | 1.32E−17 | 2.79E−13 | protein coding | HMAX |
| 2 | TSPAN33 | 1.441624756 | 2.59E−08 | 0.000273373 | protein coding | tetraspanin 33 |
| 3 | LTF | 3.403239928 | 1.41E−06 | 0.008603064 | protein coding | lactotransferrin |
| 4 | G0S2 | 1.50300118 | 1.89E−06 | 0.008603064 | protein coding | G0/G1 switch 2 |
| 5 | RTN1 | 2.058299101 | 2.11E−06 | 0.008603064 | protein coding | reticulon 1 |
| 6 | SHROOM4 | 2.156006671 | 2.44E−06 | 0.008603064 | protein coding | shroom family member 4 |
| 7 | FCN1 | 1.569170022 | 3.56E−06 | 0.009534198 | protein coding | ficolin 1 |
| 8 | PADI2 | 2.195336963 | 4.51E−06 | 0.009534198 | protein coding | peptidyl arginine deiminase 2 |
| 9 | MPP1 | 1.025128729 | 4.29E−06 | 0.009534198 | protein coding | membrane palmitoylated protein 1 |
| 10 | PROS1 | 1.943637848 | 3.72E−06 | 0.009534198 | protein coding | Protein S |

CB (CR, n = 21; PR, n = 27) vs. NCB (PD, n = 59)

| | | | | | | |
|---|---|---|---|---|---|---|
| 1 | HMAX | 4.237953364 | 1.31E−18 | 3.03E−14 | protein coding | MT-RNR2 like 8 |
| 2 | SHROOM4 | 2.267730422 | 1.08E−06 | 0.012498032 | protein coding | shroom family member 4 |
| 3 | RTN1 | 2.106095618 | 1.77E−06 | 0.013711018 | protein coding | reticulon 1 |
| 4 | G0S2 | 1.486499348 | 2.70E−06 | 0.014977281 | protein coding | G0/G1 switch 2 |
| 5 | CLCN5 | 1.401975596 | 3.23E−06 | 0.014977281 | protein coding | chloride voltage-gated channel 5 |
| 6 | HOMER2 | 1.298055409 | 5.14E−06 | 0.019879051 | protein coding | homer scaffold protein 2 |
| 7 | TSPAN9 | 1.706161799 | 7.92E−06 | 0.022134117 | protein coding | tetraspanin 9 |
| 8 | TSPAN33 | 1.003964191 | 7.08E−06 | 0.022134117 | protein coding | tetraspanin 33 |
| 9 | EHD3 | 1.088502844 | 1.33E−05 | 0.030927829 | protein coding | EH domain containing 3 |
| 10 | PTGIR | 1.866182294 | 1.54E−05 | 0.032439464 | protein coding | prostaglandin I2 receptor |

CB (CR, n = 21) vs. NCB (PD, n = 59)

| | | | | | | |
|---|---|---|---|---|---|---|
| 1 | HMAX | 4.15500316 | 7.57E−10 | 1.75E−05 | protein coding | HMAX |
| 2 | CDC42BPA | 1.595030251 | 6.64E−06 | 0.076675514 | protein coding | CDC42 binding protein kinase |
| | | | | | | alpha |
| 3 | MEF2C | 1.292225447 | 6.14E−05 | 0.159891137 | protein coding | myocyte enhancer factor 2C |
| 4 | GRK3 | 1.044256851 | 2.38E−05 | 0.159891137 | protein coding | G protein-coupled receptor kinase |
| | | | | | | 3 |
| 5 | MYOM1 | 1.137032235 | 0.000124778 | 0.159891137 | protein coding | myomesin 1 |
| 6 | ARHGEF17 | 1.349159733 | 0.000140873 | 0.159891137 | protein coding | Rho guanine nucleotide exchange |
| | | | | | | factor 17 |
| 7 | FRK | 1.911875303 | 8.02E−05 | 0.159891137 | protein coding | fyn related Src family tyrosine |
| | | | | | | kinase |
| 8 | FFAR2 | 3.363590635 | 4.84E−05 | 0.159891137 | protein coding | free fatty acid receptor 2 |
| 9 | FCGR2A | 1.909027655 | 3.56E−05 | 0.159891137 | protein coding | Fc fragment of IgG receptor IIa |
| 10 | FARP1 | 1.750862825 | 9.97E−05 | 0.159891137 | protein coding | FERM, ARH/RhoGEF and |
| | | | | | | pleckstrin domain protein 1 |

TABLE 2
Results of CD8+ T-cell differential expression analysis of:
A. Discovery Cohort of patients treated by combination Ipilimumab/Nivolumab ICI (n = 69) (Complete Response
(CR), n = 16; Partial Response (PR), n = 25; Stable Disease (SD), n = 9 Progressive Disease (PD), n = 19).
B. Validation Cohort of patients treated by combination Ipilimumab/Nivolumab ICI
(n = 46) (CR, n = 10; PR, n = 17; SD, n = 6; PD, n = 13).
C. Meta-Analysis (Discovery and Validation Cohorts) of patients treated by combination
Ipilimumab/Nivolumab ICI (n = 115) (CR, n = 26; PR, n = 42; SD, n = 15; PD, n = 32).
The expression is derived from RNA-seq analysis of baseline (pre-treatment) peripheral
blood CD8+ T-cells. The table lists the top 10 genes that are most significantly
differentially expressed (p < 0.01, |log₂FC| > 1) in each response comparison.

| | | | | | | |
|---|---|---|---|---|---|---|
| Rank | Gene | log₂FoldChange | p-value | FDR adj. p | gene biotype | description |

A. IPI/NIVO RNA-seq Discovery Cohort, n = 69
CB (CR, n = 16; PR, n = 25) vs NCB (SD, n = 9; PD, n = 19)

| | | | | | | |
|---|---|---|---|---|---|---|
| 1 | HMAX | 4.966551831 | 9.16E−19 | 2.00E−14 | protein coding | HMAX |
| 2 | S100A8 | 2.456008677 | 1.07E−06 | 0.0092445 | protein coding | S100 calcium binding |
| | | | | | | protein A8 |
| 3 | S100A9 | 2.218862956 | 1.27E−06 | 0.0092445 | protein coding | S100 calcium binding |
| | | | | | | protein A9 |
| 4 | FCGR1A | 3.837487592 | 6.77E−06 | 0.0368785 | protein coding | Fc fragment of IgG, high |
| | | | | | | affinity Ia, receptor (CD64) |
| 5 | OPRM1 | 3.286355219 | 1.16E−05 | 0.0504726 | protein coding | opioid receptor mu 1 |
| 6 | TMPRSS4 | 3.939884333 | 2.77E−05 | 0.0670619 | protein coding | Transmembrane protease, |
| | | | | | | serine 4 |
| 7 | FHIT | 1.5199198 | 2.65E−05 | 0.0670619 | protein coding | fragile histidine triad |
| | | | | | | diadenosine triphosphatase |
| 8 | GRM4 | 2.661142111 | 4.40E−05 | 0.0799335 | protein coding | glutamate receptor, |
| | | | | | | metabotropic 4 |
| 9 | MT-CO2 | 1.091799821 | 4.40E−05 | 0.0799335 | protein coding | mitochondrially encoded |
| | | | | | | cytochrome c oxidase II |
| 10 | SLC1A2 | 2.183350547 | 7.34E−05 | 0.1142566 | protein coding | solute carrier family 1 |
| | | | | | | member 2 |

CB (CR, n = 16; PR, n = 25) vs NCB (PD, n = 19)

| | | | | | | |
|---|---|---|---|---|---|---|
| 1 | HMAX | 4.297722354 | 6.15E−14 | 9.32E−10 | protein coding | HMAX |
| 2 | S100A8 | 2.850908847 | 2.44E−08 | 0.0001234 | protein coding | S100 calcium binding |
| | | | | | | protein A8 |
| 3 | S100A9 | 2.590726446 | 2.15E−08 | 0.0001234 | protein coding | S100 calcium binding |
| | | | | | | protein A9 |
| 4 | KCNH1 | 5.916300996 | 1.36E−06 | 0.0041346 | protein coding | potassium voltage-gated |
| | | | | | | channel subfamily H |
| | | | | | | member 1 |
| 5 | FHIT | 1.843480915 | 2.31E−06 | 0.0048356 | protein coding | fragile histidine triad |
| | | | | | | diadenosine triphosphatase |
| 6 | PLBD1 | 1.913572208 | 2.80E−05 | 0.0256695 | protein coding | phospholipase B domain |
| | | | | | | containing 1 |
| 7 | C5AR1 | 1.931220639 | 2.88E−05 | 0.0256695 | protein coding | complement C5a receptor 1 |
| 8 | HK2 | 1.096253177 | 3.64E−05 | 0.0264168 | protein coding | hexokinase 2 |
| 9 | MNDA | 2.061980341 | 4.56E−05 | 0.0276352 | protein coding | myeloid cell nuclear |
| | | | | | | differentiation antigen |
| 10 | SLC11A1 | 1.73512988 | 4.81E−05 | 0.0280181 | protein coding | solute carrier family 11 |
| | | | | | | member 1 |

B. IPI/NIVO RNA-seq Validation Cohort, n = 46
CB (CR, n = 10; PR, n = 17) vs NCB (SD, n = 6; PD, n = 13)

| | | | | | | |
|---|---|---|---|---|---|---|
| 1 | HMAX | 3.693479185 | 6.45E−11 | 1.40E−06 | protein coding | HMAX |
| 2 | RASGRF2- | −3.704948821 | 1.34E−06 | 0.0145572 | lncRNA | RASGRF2 antisense RNA 1 |
| | AS1 | | | | | |
| 3 | HLA-DRB5 | 2.385559837 | 9.22E−06 | 0.0667842 | protein coding | major histocompatibility |
| | | | | | | complex, class II, DR beta 5 |
| 4 | FLRT2 | −4.370790275 | 2.37E−05 | 0.1070885 | protein coding | fibronectin leucine rich |
| | | | | | | transmembrane protein 2 |
| 5 | PAX6 | −3.605357357 | 7.77E−05 | 0.126297 | protein coding | Paired box protein Pax-6 |
| 6 | BCAT1 | 1.588074398 | 7.30E−05 | 0.126297 | protein coding | branched chain amino acid |
| | | | | | | transaminase 1 |
| 7 | EFNA3 | 1.873762135 | 5.46E−05 | 0.126297 | protein coding | ephrin A3 |
| 8 | SPTBN4 | −1.853123322 | 5.89E−05 | 0.126297 | protein coding | spectrin beta, non- |
| | | | | | | erythrocytic 4 |
| 9 | DENND5B | 2.626494392 | 3.77E−05 | 0.126297 | protein coding | DENN domain containing |
| | | | | | | 5B |
| 10 | GJC1 | −3.213283995 | 8.14E−05 | 0.126297 | protein coding | gap junction protein, gamma |
| | | | | | | 1 |

CB (CR, n = 10; PR, n = 17) vs NCB (PD, n = 13)

| | | | | | | |
|---|---|---|---|---|---|---|
| 1 | HMAX | 4.196858127 | 4.08E−12 | 8.13E−08 | protein coding | HMAX |
| 2 | TF | −3.808153615 | 2.52E−07 | 0.002507 | protein coding | transferrin |
| 3 | RP11- | −1.973951765 | 5.68E−06 | 0.0376955 | unprocessed | M-phase phosphoprotein 10 |
| | 632K20.2 | | | | pseudogene | (MPHOSPH10) pseudogene |
| 4 | BCAT1 | 1.890956587 | 1.97E−05 | 0.0580939 | protein coding | branched chain amino acid |
| | | | | | | transaminase 1 |
| 5 | SRGAP1 | −2.747506699 | 2.04E−05 | 0.0580939 | protein coding | SLIT-ROBO Rho GTPase |
| | | | | | | activating protein 1 |
| 6 | AC099661.1 | 4.585930226 | 1.51E−05 | 0.0580939 | lncRNA | novel transcript |
| 7 | AL096711.2 | −1.606257776 | 2.45E−05 | 0.0608837 | protein coding | novel protein |
| 8 | RAPGEF3 | −1.956527908 | 5.40E−05 | 0.0849395 | protein coding | Rap guanine nucleotide |
| | | | | | | exchange factor 3 |
| 9 | PLCB4 | −2.602793463 | 5.04E−05 | 0.0849395 | protein coding | 1-phosphatidylinositol 4,5- |
| | | | | | | bisphosphate |
| 10 | OTOF | −2.353715485 | 4.77E−05 | 0.0849395 | protein coding | phosphodiesterase beta-4 |
| | | | | | | Otoferlin |

C. IPI/NIVO RNA-seq Meta-Analysis, n = 115
CB (CR, n = 26; PR, n = 42) vs NCB (SD, n = 15; PD, n = 32)

| | | | | | | |
|---|---|---|---|---|---|---|
| 1 | HMAX | 4.953431843 | 1.36E−32 | 3.12E−28 | protein coding | HMAX |
| 2 | S100A9 | 2.574907212 | 8.26E−12 | 9.49E−08 | protein coding | S100 calcium binding |
| | | | | | | protein A9 |
| 3 | S100A8 | 1.977407428 | 3.38E−07 | 0.0025925 | protein coding | S100 calcium binding |
| | | | | | | protein A8 |
| 4 | VCAN | 1.728222061 | 3.12E−06 | 0.0175438 | protein coding | versican |
| 5 | FLRT2 | −3.20174567 | 3.82E−06 | 0.0175438 | protein coding | fibronectin leucine rich |
| | | | | | | transmembrane protein 2 |
| 6 | DNAH11 | −2.29648695 | 7.82E−06 | 0.0275913 | protein coding | dynein axonemal heavy |
| | | | | | | chain 11 |
| 7 | PADI2 | 2.142975507 | 1.06E−05 | 0.0275913 | protein coding | peptidyl arginine deiminase |
| | | | | | | 2 |
| 8 | BRDT | −1.857179141 | 9.44E−06 | 0.0275913 | protein coding | bromodomain, testis-specific |
| 9 | S100A12 | 2.291381363 | 1.32E−05 | 0.0275913 | protein coding | S100 calcium binding |
| | | | | | | protein A12 |
| 10 | MNDA | 1.565055378 | 1.24E−05 | 0.0275913 | protein coding | myeloid cell nuclear |
| | | | | | | differentiation antigen |

CB (CR, n = 26; PR, n = 42) vs NCB (PD, n = 32)

| | | | | | | |
|---|---|---|---|---|---|---|
| 1 | HMAX | 4.819378762 | 3.83E−29 | 6.50E−25 | protein coding | HMAX |
| 2 | S100A9 | 2.996357403 | 2.51E−14 | 2.13E−10 | protein coding | S100 calcium binding |
| | | | | | | protein A9 |
| 3 | FCN1 | 2.519162482 | 3.68E−12 | 2.08E−08 | protein coding | ficolin 1 [Source: HGNC |
| | | | | | | Symbol; Acc: HGNC: 3623] |
| 4 | S100A8 | 2.314132341 | 1.87E−08 | 7.93E−05 | protein coding | S100 calcium binding |
| | | | | | | protein A8 |
| 5 | VCAN | 2.144782057 | 6.58E−08 | 0.0002232 | protein coding | versican |
| 6 | SERPINA1 | 1.75020927 | 1.98E−07 | 0.0005605 | protein coding | serpin family A member 1 |
| 7 | NGFRAP1 | 1.165972407 | 2.58E−07 | 0.0006255 | protein coding | nerve growth factor receptor |
| | | | | | | (TNFRSF16) associated |
| | | | | | | protein 1 |
| 8 | LYZ | 1.773591843 | 4.96E−07 | 0.0007918 | protein coding | lysozyme |
| 9 | S100A12 | 2.838544579 | 5.14E−07 | 0.0007918 | protein coding | S100 calcium binding |
| | | | | | | protein A12 |
| 10 | C5AR1 | 1.740576074 | 4.94E−07 | 0.0007918 | protein coding | complement C5a receptor 1 |

CB (CR, n = 26) vs NCB (PD, n = 32)

| | | | | | | |
|---|---|---|---|---|---|---|
| 1 | HMAX | 4.904365273 | 6.90E−15 | 6.48E−11 | protein coding | HMAX |
| 2 | HK2 | 1.248898089 | 2.55E−06 | 0.0065649 | protein coding | hexokinase 2 |
| 3 | ADD2 | 1.200353304 | 8.27E−05 | 0.0707151 | protein coding | adducin 2 |
| 4 | ITGAX | −1.144619135 | 7.87E−05 | 0.0707151 | lncRNA | integrin subunit alpha X |
| 5 | FHIT | 1.693915777 | 0.000129 | 0.075879 | protein coding | fragile histidine triad |
| | | | | | | diadenosine triphosphatase |
| 6 | VCAN | 1.922832732 | 0.000259 | 0.0776774 | protein coding | versican |
| 7 | NGFRAP1 | 1.126397773 | 0.000227 | 0.0776774 | protein coding | nerve growth factor receptor |
| | | | | | | (TNFRSF16) associated |
| | | | | | | protein 1 |
| 8 | GPX1 | 1.08970375 | 0.000283 | 0.0776774 | lncRNA | glutathione peroxidase 1 |
| 9 | SERPINA1 | 1.47313709 | 0.000835 | 0.0969447 | protein coding | serpin family A member 1 |
| 10 | AC007278.1 | −1.154800346 | 0.001267 | 0.1042573 | lncRNA | novel transcript |

TABLE 3
Results of CD8+ T-cell differential chromatin accessibility analysis from:
A. Discovery Cohort of patients treated by single-agent Nivolumab ICI (n = 70) (Complete Response
(CR), n = 14; Partial Response (PR), n = 15; Stable Disease (SD), n = 6 Progressive Disease (PD), n = 35).
B. Validation Cohort of patients treated by single-agent Nivolumab
ICI (n = 46) (CR, n = 7; PR, n = 12) vs. NCB (SD, n = 3; PD, n = 24).
C. Meta-Analysis (Discovery and Validation Cohorts) of patients treated by single-agent
Nivolumab ICI (n = 116) (CR, n = 21; PR, n = 28) vs. NCB (SD, n = 11; PD, n = 60).
The chromatin accessibility is derived from ATAC-seq analysis of baseline (pre-treatment) peripheral blood CD8+
T-cells. The table lists the top 10 chromatin regions that are most significantly differentially accessible (p <
0.05) in each response comparison. All chromsomal positions are based on human genome build Human GRCh38 (hg38).

| | | | | | | | | | | |
|---|---|---|---|---|---|---|---|---|---|---|
| | | | | | | Mean | Mean | | | |
| | | | | | Mean | Accessibility | Accessibility | | | |
| Rank | Chromosome | Start | End | Width | accessibility | CB | NCB | Fold | p-value | FDR adj. p |

A. NIVO ATAC-seq Discovery Cohort, n = 70
CB (CR, n = 14; PR, n = 15) vs. NCB (SD, n = 6; PD, n = 35)

| | | | | | | | | | | |
|---|---|---|---|---|---|---|---|---|---|---|
| 1 | chrX | 126472363 | 126473271 | 909 | 5.58 | 3.91 | 6.15 | −2.24 | 1.18E−15 | 1.03E−10 |
| 2 | chr10 | 9996 | 10430 | 435 | 0.95 | −0.95 | 1.55 | −2.5 | 4.65E−12 | 2.03E−07 |
| 3 | chr10 | 10654837 | 10655483 | 647 | 0.83 | −0.81 | 1.39 | −2.2 | 1.14E−10 | 3.30E−06 |
| 4 | chr3 | 96617245 | 96618433 | 1189 | 6.66 | 7.39 | 5.75 | 1.64 | 1.29E−09 | 2.80E−05 |
| 5 | chr14 | 99419499 | 99420981 | 1483 | 6.8 | 6.08 | 7.15 | −1.07 | 7.17E−09 | 0.000125 |
| 6 | chr8 | 123864127 | 123864571 | 445 | 0.44 | −0.93 | 0.96 | −1.89 | 2.60E−08 | 0.000378 |
| 7 | chr5 | 134923786 | 134924739 | 954 | 7.61 | 6.28 | 8.13 | −1.85 | 5.72E−08 | 0.000712 |
| 8 | chr3 | 157879844 | 157880120 | 277 | 2.42 | 1.52 | 2.82 | −1.31 | 3.22E−07 | 0.0034 |
| 9 | chr18 | 80262779 | 80263269 | 491 | 1.52 | 0.3 | 2.01 | −1.72 | 3.84E−07 | 0.0034 |
| 10 | chr11 | 10508542 | 10509994 | 1453 | 6.27 | 5.28 | 6.71 | −1.43 | 4.18E−07 | 0.0034 |

CB (CR, n = 14; PR, n = 15) vs. NCB (PD, n = 35)

| | | | | | | | | | | |
|---|---|---|---|---|---|---|---|---|---|---|
| 1 | chr3 | 96617245 | 96618433 | 1189 | 6.81 | 7.63 | 5.39 | 2.23 | 1.27E−18 | 1.08E−13 |
| 2 | chrX | 126472363 | 126473270 | 908 | 5.9 | 4.15 | 6.56 | −2.42 | 5.68E−17 | 2.40E−12 |
| 3 | chr5 | 134923786 | 134924694 | 909 | 7.96 | 6.5 | 8.57 | −2.06 | 3.73E−09 | 0.000105 |
| 4 | chr11 | 10508542 | 10509993 | 1452 | 6.6 | 5.51 | 7.12 | −1.61 | 3.74E−08 | 0.00079 |
| 5 | chr10 | 9996 | 10430 | 435 | 0.59 | −0.72 | 1.17 | −1.89 | 6.46E−08 | 0.00109 |
| 6 | chr17 | 36213002 | 36213300 | 299 | 3.69 | 4.06 | 3.29 | 0.77 | 2.15E−07 | 0.00303 |
| 7 | chr10 | 10654837 | 10655480 | 644 | 0.56 | −0.57 | 1.09 | −1.66 | 4.30E−07 | 0.00519 |
| 8 | chr21 | 5355319 | 5364232 | 8914 | 3.73 | 3.03 | 4.14 | −1.11 | 6.71E−07 | 0.00708 |
| 9 | chr21 | 35457210 | 35457535 | 326 | 2.43 | 2.82 | 1.99 | 0.83 | 4.32E−06 | 0.0405 |
| 10 | chrX | 87546486 | 87547013 | 528 | 2.68 | 2.28 | 2.93 | −0.65 | 9.89E−06 | 0.0835 |

B. NIVO ATAC-seq Validation Cohort, n = 46
CB (CR, n = 7; PR, n = 12) vs. NCB (SD, n = 3; PD, n = 24)

| | | | | | | | | | | |
|---|---|---|---|---|---|---|---|---|---|---|
| 1 | chr | 630286 | 631368 | 1083 | 9.8 | 7.44 | 10.45 | −3.01 | 1.23E−11 | 1.48E−06 |
| 2 | chr11 | 10508201 | 10509996 | 1796 | 6.35 | 4.98 | 6.87 | −1.89 | 8.43E−10 | 5.07E−05 |
| 3 | chr21 | 44138161 | 44138672 | 512 | 1.03 | 1.81 | 0.03 | 1.78 | 8.34E−09 | 0.000334 |
| 4 | chr21 | 44142993 | 44144801 | 1809 | 2.48 | 3.34 | 1.26 | 2.07 | 6.93E−08 | 0.00208 |
| 5 | chr1 | 101370924 | 101371524 | 601 | 4.25 | 4.64 | 3.9 | 0.73 | 1.17E−07 | 0.00282 |
| 6 | chr2 | 225308519 | 225309297 | 779 | 4.3 | 4.58 | 4.06 | 0.52 | 1.87E−07 | 0.00327 |
| 7 | chr2 | 65027065 | 65027405 | 341 | 2.99 | 2.33 | 3.32 | −1.0 | 1.90E−07 | 0.00327 |
| 8 | chrY | 9506636 | 9506948 | 313 | 1.17 | 1.85 | 0.37 | 1.48 | 2.86E−07 | 0.0043 |
| 9 | chr3 | 3573220 | 3574148 | 929 | 4.8 | 5.1 | 4.55 | 0.56 | 4.36E−07 | 0.00583 |
| 10 | chr1 | 26136768 | 26138340 | 1573 | 3.64 | 3.18 | 3.9 | −0.72 | 9.72E−07 | 0.0117 |

CB (CR, n = 7; PR, n = 12) vs. NCB (PD, n = 24)

| | | | | | | | | | | |
|---|---|---|---|---|---|---|---|---|---|---|
| 1 | chrY | 9506636 | 9506948 | 313 | 1.15 | 1.87 | 0.08 | 1.8 | 3.00E−10 | 3.66E−05 |
| 2 | chr20 | 1588225 | 1589116 | 892 | 3.71 | 4.55 | 2.27 | 2.27 | 7.74E−08 | 0.00472 |
| 3 | chr20 | 19284131 | 19284746 | 616 | 4.3 | 4.59 | 4.01 | 0.59 | 1.76E−07 | 0.00716 |
| 4 | chr4 | 49297201 | 49299369 | 2169 | 0.98 | 1.52 | 0.32 | 1.21 | 3.71E−07 | 0.0113 |
| 5 | chr8 | 58309472 | 58310363 | 892 | 4.58 | 4.87 | 4.3 | 0.57 | 5.08E−07 | 0.0124 |
| 6 | chrX | 89202927 | 89207888 | 4962 | 4.02 | 3.62 | 4.27 | −0.65 | 7.40E−07 | 0.0126 |
| 7 | chr5 | 178585453 | 178585741 | 289 | 6.03 | 5.57 | 6.32 | −0.75 | 8.02E−07 | 0.0126 |
| 8 | chr20 | 22828543 | 22829220 | 678 | 4.48 | 4.73 | 4.24 | 0.5 | 8.29E−07 | 0.0126 |
| 9 | chr5 | 116452916 | 116453425 | 510 | 4.24 | 4.49 | 4.02 | 0.47 | 1.28E−06 | 0.0155 |
| 10 | chr5 | 8879519 | 8880669 | 1151 | 5.14 | 5.33 | 4.96 | 0.37 | 1.50E−06 | 0.0155 |
| | \\ | | | | | | | | | |
| 193 | chr11 | 10508198 | 10509996 | 1799 | 5.42 | 4.95 | 5.71 | −0.75 | 8.54E−05 | 0.0537 |

C. NIVO ATAC-seq Meta-Analysis Cohort, n = 116
CB (CR, n = 21; PR, n = 27) vs. NCB (SD, n = 9; PD, n = 59)

| | | | | | | | | | | |
|---|---|---|---|---|---|---|---|---|---|---|
| 1 | chr11 | 10507914 | 10509996 | 2083 | 6.09 | 5.05 | 6.54 | −1.48 | 6.75E−13 | 7.86E−08 |
| 2 | chr10 | 9996 | 10430 | 435 | 0.43 | −1 | 0.96 | −1.96 | 1.02E−12 | 7.86E−08 |
| 3 | chrY | 9506636 | 9506948 | 313 | 0.24 | 0.79 | −0.34 | 1.13 | 2.78E−10 | 1.43E−05 |
| 4 | chr10 | 10654837 | 10655483 | 647 | 0.44 | −0.65 | 0.91 | −1.56 | 1.87E−09 | 7.20E−05 |
| 5 | chr14 | 99419499 | 99420981 | 1483 | 6.38 | 5.82 | 6.67 | −0.85 | 4.21E−09 | 0.000126 |
| 6 | chr5 | 134923786 | 134924739 | 954 | 7.66 | 6.51 | 8.13 | −1.62 | 4.92E−09 | 0.000126 |
| 7 | chr20 | 27460597 | 27472688 | 12092 | 2.76 | 1.82 | 3.18 | −1.36 | 1.37E−08 | 0.000302 |
| 8 | chrX | 144668451 | 144669276 | 826 | 2.86 | 2.46 | 3.08 | −0.63 | 4.38E−08 | 0.000845 |
| 9 | chr17 | 36212925 | 36213413 | 489 | 3.72 | 4.08 | 3.39 | 0.69 | 1.44E−07 | 0.00246 |
| 10 | chrY | 11781410 | 11781758 | 349 | 0.88 | 1.36 | 0.41 | 0.95 | 2.01E−07 | 0.00311 |

CB (CR, n = 21; PR, n = 27) vs. NCB ( PD, n = 59)

| | | | | | | | | | | |
|---|---|---|---|---|---|---|---|---|---|---|
| 1 | chr11 | 10507916 | 10509996 | 2081 | 6.38 | 5.29 | 6.9 | −1.61 | 5.72E−14 | 8.43E−09 |
| 2 | chr5 | 134923786 | 134924694 | 909 | 7.98 | 6.74 | 8.53 | −1.79 | 3.69E−10 | 2.72E−05 |
| 3 | chr17 | 36212925 | 36213413 | 489 | 3.94 | 4.32 | 3.54 | 0.78 | 8.02E−09 | 0.000394 |
| 4 | chr20 | 27460597 | 27463030 | 2434 | 2.71 | 1.66 | 3.21 | −1.55 | 2.40E−08 | 0.000885 |
| 5 | chr7 | 63829616 | 63831474 | 1859 | 3.87 | 4.04 | 3.72 | 0.32 | 1.56E−07 | 0.00409 |
| 6 | chr16 | 73334011 | 73334565 | 555 | 4.05 | 4.24 | 3.87 | 0.37 | 1.90E−07 | 0.00409 |
| 7 | chr9 | 118230252 | 118230910 | 659 | 3.72 | 3.91 | 3.54 | 0.37 | 1.94E−07 | 0.00409 |
| 8 | chrY | 11781410 | 11781758 | 349 | 1.14 | 1.59 | 0.63 | 0.97 | 2.98E−07 | 0.00549 |
| 9 | chr13 | 80063194 | 80063651 | 458 | 3 | 3.21 | 2.8 | 0.41 | 6.52E−07 | 0.00962 |
| 10 | chr3 | 3683879 | 3685072 | 1194 | 4.88 | 5.03 | 4.75 | 0.28 | 6.52E−07 | 0.00962 |

CB (CR, n = 21) vs. NCB ( PD, n = 59)

| | | | | | | | | | | |
|---|---|---|---|---|---|---|---|---|---|---|
| 1 | chrX | 146619661 | 146620596 | 936 | 2.78 | 2.07 | 2.98 | −0.91 | 1.08E−07 | 0.00734 |
| 2 | chr8 | 58309472 | 58310634 | 1163 | 4.25 | 4.59 | 4.11 | 0.48 | 1.16E−07 | 0.00734 |
| 3 | chr7 | 75179900 | 75180804 | 905 | −0.21 | 0.51 | −0.6 | 1.11 | 2.17E−07 | 0.00915 |
| 4 | chr17 | 36212925 | 36213413 | 489 | 3.97 | 4.56 | 3.68 | 0.88 | 4.29E−07 | 0.0136 |
| 5 | chr8 | 58360770 | 58361608 | 839 | 4.2 | 4.6 | 4.02 | 0.57 | 9.10E−07 | 0.0209 |
| 6 | chr12 | 76985090 | 76985542 | 453 | 2.69 | 2.25 | 2.83 | −0.58 | 1.09E−06 | 0.0209 |
| 7 | chr15 | 22253285 | 22254838 | 1554 | 6.05 | 6.42 | 5.88 | 0.54 | 1.15E−06 | 0.0209 |
| 8 | chr5 | 107519735 | 107520744 | 1010 | 5.01 | 5.37 | 4.86 | 0.51 | 1.80E−06 | 0.0285 |
| 9 | chr15 | 20353157 | 20354538 | 1382 | 6.5 | 6.87 | 6.34 | 0.53 | 2.23E−06 | 0.0302 |
| 10 | chrX | 18404751 | 18405223 | 473 | 1.67 | 0.94 | 1.87 | −0.93 | 2.67E−06 | 0.0302 |
| | \\ | | | | | | | | | |
| 580 | chr11 | 10507916 | 10509996 | 2081 | 6.44 | 5.69 | 6.64 | −0.95 | 0.0011 | 0.239 |

TABLE 4
Results of CD8+ T-cell differential chromatin accessibility analysis from:
A. Discovery Cohort of patients treated by combination Ipilimumab/Nivolumab ICI (n = 62) (Complete
Response (CR), n = 13; Partial Response (PR), n = 25; Stable Disease (SD), n = 9; Progressive Disease (PD), n = 15).
B. Validation Cohort of patients treated by combination Ipilimumab/Nivolumab
ICI (n = 48) (CR, n = 10; PR, n = 17) vs. NCB (SD, n = 7; PD, n = 14).
C. Meta-Analysis (Discovery and Validation Cohorts) of patients treated by combination
Ipilimumab/Nivolumab ICI (n = 110) (CR, n = 23; PR, n = 42) vs. NCB (SD, n = 16; PD, n = 29).
The chromatin accessibility is derived from ATAC-seq analysis of baseline (pre-treatment) peripheral blood CD8+
T-cells. The table lists the top 20 chromatin regions that are most significantly differentially accessible (p <
0.05) in each response comparison. All chromsomal positions are based on human genome build Human GRCh38 (hg38).

| | | | | | | | | | | |
|---|---|---|---|---|---|---|---|---|---|---|
| | | | | | | Mean | Mean | | | |
| | | | | | Mean | Accessibility | Accessibility | | | |
| Rank | Chromosome | Start | End | Width | accessibility | CB | NCB | Fold | p-value | FDR adj. p |

A. IPI/NIVO ATAC-seq Discovery Cohort, n = 62
CB (CR, n = 13; PR, n = 25) vs. NCB (SD, n = 9; PD, n = 15)

| | | | | | | | | | | |
|---|---|---|---|---|---|---|---|---|---|---|
| 1 | chr2 | 46369712 | 46370382 | 671 | 4.69 | 4.97 | 4.07 | 0.91 | 6.81E−17 | 3.47E−12 |
| 2 | chr1 | 93758517 | 93759524 | 1008 | 5.21 | 5.47 | 4.67 | 0.79 | 2.28E−13 | 5.80E−09 |
| 3 | chr5 | 80650506 | 80652039 | 1534 | 6.71 | 5.48 | 7.64 | −2.16 | 2.36E−12 | 4.00E−08 |
| 4 | chrX | 126472163 | 126473271 | 1109 | 5.29 | 4.21 | 6.17 | −1.97 | 7.76E−12 | 9.87E−08 |
| 5 | chr1 | 6293961 | 6295281 | 1321 | 5.42 | 5.68 | 4.87 | 0.8 | 1.27E−11 | 1.30E−07 |
| 6 | chr14 | 51596167 | 51597163 | 997 | 4.61 | 4.85 | 4.13 | 0.72 | 3.84E−11 | 3.26E−07 |
| 7 | chr3 | 4748650 | 4749475 | 826 | 3.97 | 4.21 | 3.49 | 0.72 | 6.32E−11 | 3.97E−07 |
| 8 | chr2 | 28508255 | 28508974 | 720 | 5.12 | 5.38 | 4.57 | 0.8 | 6.32E−11 | 3.97E−07 |
| 9 | chr19 | 54905695 | 54907066 | 1372 | 4.93 | 5.15 | 4.49 | 0.65 | 7.02E−11 | 3.97E−07 |
| 10 | chr10 | 110459869 | 110461229 | 1361 | 5.3 | 5.5 | 4.91 | 0.59 | 3.67E−10 | 1.87E−06 |
| | \\ | | | | | | | | | |
| 20 | chr11 | 10508160 | 10509996 | 1837 | 5.65 | 4.91 | 6.35 | −1.45 | 8.34E−10 | 2.12E−06 |

CB (CR, n = 13; PR, n = 25) vs. NCB (PD, n = 15)

| | | | | | | | | | | |
|---|---|---|---|---|---|---|---|---|---|---|
| 1 | chr2 | 46369712 | 46370382 | 671 | 4.76 | 4.97 | 3.99 | 0.98 | 2.24E−13 | 1.19E−08 |
| 2 | chrX | 126472163 | 126473271 | 1109 | 5.26 | 4.21 | 6.47 | −2.27 | 4.43E−13 | 1.19E−08 |
| 3 | chr5 | 80650506 | 80652039 | 1534 | 6.66 | 5.48 | 7.93 | −2.45 | 1.43E−12 | 2.56E−08 |
| 4 | chr7 | 66124802 | 66126711 | 1910 | 5.49 | 5.75 | 4.52 | 1.23 | 1.03E−10 | 1.38E−06 |
| 5 | chr1 | 93758517 | 93759524 | 1008 | 5.27 | 5.47 | 4.63 | 0.83 | 4.48E−10 | 4.23E−06 |
| 6 | chr10 | 110459869 | 110461229 | 1361 | 5.34 | 5.5 | 4.8 | 0.7 | 4.73E−10 | 4.23E−06 |
| 7 | chr17 | 3693852 | 3694293 | 442 | 3.95 | 4.14 | 3.35 | 0.79 | 5.90E−10 | 4.53E−06 |
| 8 | chr1 | 6293961 | 6295281 | 1321 | 5.48 | 5.68 | 4.8 | 0.88 | 9.16E−10 | 6.14E−06 |
| 9 | chr7 | 139886098 | 139886705 | 608 | 3.93 | 4.11 | 3.32 | 0.79 | 2.26E−09 | 1.35E−05 |
| 10 | chr3 | 4748650 | 4749475 | 826 | 4.03 | 4.21 | 3.43 | 0.79 | 3.36E−09 | 0.000018 |
| \\ | | | | | | | | | | |
| 29 | chr11 | 10508160 | 10509996 | 1837 | 5.49 | 4.91 | 6.38 | −1.47 | 3.59E−08 | 6.57E−05 |

B. COMBO ATAC-seq Validation Cohort, n = 48
CB (CR, n = 10; PR, n = 17) vs. NCB (SD, n = 7; PD, n = 14)

| | | | | | | | | | | |
|---|---|---|---|---|---|---|---|---|---|---|
| 1 | chr1 | 234314884 | 234315819 | 936 | 4.74 | 5.15 | 3.95 | 1.2 | 5.75E−07 | 2.40E−02 |
| 2 | chrX | 126472360 | 126473271 | 912 | 6.16 | 4.96 | 6.95 | −1.99 | 6.30E−07 | 2.40E−02 |
| 3 | chr5 | 80650505 | 80651993 | 1489 | 7.17 | 6.11 | 7.91 | −1.81 | 2.95E−06 | 7.49E−02 |
| 4 | chr13 | 18995117 | 18995749 | 633 | 3.89 | 3.64 | 4.16 | −0.51 | 8.62E−06 | 1.45E−01 |
| 5 | chr3 | 14216276 | 14216968 | 693 | 4.13 | 4.44 | 3.6 | 0.84 | 9.50E−06 | 1.45E−01 |
| 6 | chr6 | 87915022 | 87915455 | 434 | 3.36 | 2.95 | 3.75 | −0.8 | 1.58E−05 | 1.75E−01 |
| 7 | chr8 | 42926351 | 42926943 | 593 | 3.91 | 3.66 | 4.18 | −0.52 | 1.61E−05 | 1.75E−01 |
| 8 | chr7 | 44051097 | 44052080 | 984 | 3.73 | 3.52 | 3.96 | −0.44 | 1.96E−05 | 1.82E−01 |
| 9 | chr18 | 26195406 | 26196192 | 787 | 2.35 | 2.61 | 1.92 | 0.69 | 2.15E−05 | 1.82E−01 |
| 10 | chr5 | 257282 | 262414 | 5133 | 2.84 | 2.56 | 3.13 | −0.57 | 2.66E−05 | 2.03E−01 |
| \\ | | | | | | | | | | |
| 439 | chr11 | 10508199 | 10509996 | 1798 | 7.17 | 6.58 | 7.68 | −1.09 | 6.90E−03 | 9.04E−01 |

CB (CR, n = 10; PR, n = 17) vs. NCB (PD, n = 14)

| | | | | | | | | | | |
|---|---|---|---|---|---|---|---|---|---|---|
| 1 | chr1 | 234314884 | 234315819 | 936 | 5.12 | 5.47 | 4.04 | 1.43 | 9.37E−08 | 4.68E−03 |
| 2 | KI270733.1 | 121318 | 125255 | 3938 | 6.08 | 6.39 | 5.19 | 1.2 | 1.84E−07 | 4.68E−03 |
| 3 | chrX | 126472361 | 126473270 | 910 | 6.53 | 5.28 | 7.61 | −2.33 | 2.03E−07 | 4.68E−03 |
| 4 | chr17 | 22524064 | 22524530 | 467 | 9.75 | 10.23 | 7.71 | 2.53 | 1.58E−06 | 2.73E−02 |
| 5 | chr5 | 80650506 | 80651991 | 1486 | 7.48 | 6.43 | 8.49 | −2.06 | 2.86E−06 | 3.26E−02 |
| 6 | chr2 | 62443332 | 62444092 | 761 | 3.84 | 4.02 | 3.43 | 0.59 | 3.20E−06 | 3.26E−02 |
| 7 | chr17 | 23693638 | 23699882 | 6245 | 1.07 | 1.51 | −0.59 | 2.1 | 3.30E−06 | 3.26E−02 |
| 8 | chr18 | 19470330 | 19481576 | 11247 | 2.3 | 1.88 | 2.87 | −0.99 | 1.53E−05 | 1.33E−01 |
| 9 | chr3 | 14216276 | 14216968 | 693 | 4.51 | 4.76 | 3.83 | 0.93 | 1.98E−05 | 1.51E−01 |
| 10 | chr7 | 44051097 | 44052080 | 984 | 4.03 | 3.84 | 4.34 | −0.49 | 2.18E−05 | 1.51E−01 |
| \\ | | | | | | | | | | |
| 104 | chr11 | 10508199 | 10509993 | 1795 | 7.62 | 6.9 | 8.44 | −1.53 | 9.90E−04 | 6.46E−01 |

C. COMBO ATAC-seq Meta-Analysis Cohort, n = 110
CB (CR, n = 23; PR, n = 42) vs. NCB (SD, n = 16; PD, n = 29)

| | | | | | | | | | | |
|---|---|---|---|---|---|---|---|---|---|---|
| 1 | chrX | 126472163 | 1.26E+08 | 1109 | 5.66 | 4.51 | 6.5 | −1.99 | 4.09E−17 | 3.03E−12 |
| 2 | chr5 | 80650505 | 80652039 | 1535 | 6.85 | 5.69 | 7.69 | −2.0 | 1.33E−16 | 4.90E−12 |
| 3 | chr2 | 46369671 | 46370382 | 712 | 4.68 | 4.87 | 4.36 | 0.5 | 1.83E−08 | 4.51E−04 |
| 4 | chr11 | 10508160 | 10509996 | 1837 | 6.39 | 5.73 | 7.01 | −1.28 | 6.32E−08 | 1.17E−03 |
| 5 | chr22 | 21689511 | 21690533 | 1023 | 4.17 | 4.35 | 3.85 | 0.51 | 1.71E−07 | 2.54E−03 |
| 6 | chr5 | 1151973 | 1152966 | 994 | 4.54 | 4.66 | 4.33 | 0.33 | 6.48E−07 | 7.99E−03 |
| 7 | chr12 | 101743121 | 1.02E+08 | 1620 | 5.34 | 5.48 | 5.12 | 0.36 | 1.11E−06 | 1.11E−02 |
| 8 | chr20 | 20181196 | 20181916 | 721 | 4.04 | 4.2 | 3.77 | 0.44 | 1.20E−06 | 1.11E−02 |
| 9 | chr18 | 58887276 | 58887912 | 637 | 3.62 | 3.73 | 3.43 | 0.3 | 1.56E−06 | 1.28E−02 |
| 10 | chr4 | 139528523 | 1.40E+08 | 655 | 4.03 | 4.19 | 3.76 | 0.43 | 2.16E−06 | 1.49E−02 |

CB (CR, n = 23; PR, n = 42) vs. NCB ( PD, n = 29)

| | | | | | | | | | | |
|---|---|---|---|---|---|---|---|---|---|---|
| 1 | chrX | 126472163 | 1.26E+08 | 1109 | 5.67 | 4.51 | 6.83 | −2.32 | 1.22E−18 | 8.69E−14 |
| 2 | chr5 | 80650506 | 80652039 | 1534 | 6.82 | 5.69 | 7.96 | −2.27 | 1.08E−16 | 3.83E−12 |
| 3 | chr11 | 10508160 | 10509996 | 1837 | 6.44 | 5.73 | 7.34 | −1.61 | 4.96E−09 | 1.17E−04 |
| 4 | chr2 | 46369671 | 46370382 | 712 | 4.71 | 4.87 | 4.28 | 0.59 | 7.21E−09 | 1.28E−04 |
| 5 | chr10 | 394203 | 395234 | 1032 | 3.99 | 4.12 | 3.66 | 0.46 | 2.40E−08 | 3.21E−04 |
| 6 | chr6 | 144197814 | 1.44E+08 | 1405 | 4.54 | 4.68 | 4.17 | 0.51 | 2.71E−08 | 3.21E−04 |
| 7 | KI270733.1 | 121311 | 125255 | 3945 | 5.68 | 5.9 | 5.01 | 0.89 | 4.97E−08 | 4.42E−04 |
| 8 | chr6 | 167121825 | 1.67E+08 | 1178 | 4.51 | 4.62 | 4.23 | 0.4 | 4.97E−08 | 4.42E−04 |
| 9 | chr5 | 1151973 | 1152966 | 994 | 4.55 | 4.66 | 4.27 | 0.4 | 7.02E−08 | 5.54E−04 |
| 10 | chr22 | 39205110 | 39206487 | 1378 | 4.77 | 4.94 | 4.32 | 0.62 | 1.62E−07 | 1.15E−03 |

CB (CR, n = 23) vs. NCB ( PD, n = 29)

| | | | | | | | | | | |
|---|---|---|---|---|---|---|---|---|---|---|
| 1 | chr5 | 80650506 | 80652039 | 1534 | 7.81 | 6.32 | 8.41 | −2.09 | 1.54E−07 | 5.45E−03 |
| 2 | chr22 | 45341482 | 45341844 | 363 | 3.98 | 3.29 | 4.36 | −1.07 | 1.88E−07 | 5.45E−03 |
| 3 | chr3 | 58355586 | 58356374 | 789 | 3.86 | 4.21 | 3.52 | 0.69 | 2.98E−07 | 5.45E−03 |
| 4 | chr11 | 102474165 | 1.02E+08 | 1013 | 3.95 | 4.31 | 3.57 | 0.74 | 3.28E−07 | 5.45E−03 |
| 5 | chr6 | 130766562 | 1.31E+08 | 728 | 3.73 | 4.01 | 3.46 | 0.54 | 7.30E−07 | 9.69E−03 |
| 6 | chr5 | 175507299 | 1.76E+08 | 666 | 4.37 | 3.94 | 4.64 | −0.7 | 9.66E−07 | 1.07E−02 |
| 7 | chrX | 126472163 | 1.26E+08 | 1109 | 6.72 | 5.4 | 7.28 | −1.88 | 1.31E−06 | 1.24E−02 |
| 8 | chr5 | 95773291 | 95773694 | 404 | 3.57 | 3.91 | 3.21 | 0.7 | 2.07E−06 | 1.35E−02 |
| 9 | chr10 | 5291433 | 5294429 | 2997 | 6.85 | 6.54 | 7.06 | −0.52 | 2.14E−06 | 1.35E−02 |
| 10 | chr | 63892419 | 63893606 | 1188 | 5.32 | 5.57 | 5.09 | 0.49 | 2.45E−06 | 1.35E−02 |
| | \\ | | | | | | | | | |
| 20 | chr11 | 10508160 | 10509996 | 1837 | 7.26 | 6.09 | 7.79 | −1.7 | 4.19E−06 | 1.35E−02 |

TABLE 5
Demographic overview for patients in the final analytic cohort

| | | CheckMate-067 Clinical Trial | | | |
|---|---|---|---|---|---|
| | | NIVO | NIVO | | COMBO |
| | Total | Discovery | Validation 1 | IPI Discovery | Discovery |
| | (N = 1,254) | (N = 115) | (N = 108) | (N = 98) | (N = 205) |
| Sex | | | | | |
| Male | 797 (63.6%) | 65 (56.5%) | 66 (61.1%) | 65 (66.3%) | 137 (66.8%) |
| Female | 457 (36.4%) | 50 (43.5%) | 42 (38.9%) | 33 (33.7%) | 68 (33.2%) |
| Age at treatment | | | | | |
| start^{b} (years) | | | | | |
| Mean (SD) | 60.1 (14.0) | 57.9 (14.7) | 58.6 (13.6) | 61.8 (14.9) | 59.2 (14.1) |
| Median [Min, Max] | 61.0 [19.0, 90.6] | 59.0 [25.0, 88.0] | 60.0 [28.0, 89.0] | 63.0 [21.0, 89.0] | 62.0 [23.0, 87.0] |
| Haplogroup | | | | | |
| H | 580 (46.3%) | 52 (45.2%) | 50 (46.3%) | 49 (50.0%) | 98 (47.8%) |
| U | 160 (12.8%) | 17 (14.8%) | 14 (13.0%) | 16 (16.3%) | 24 (11.7%) |
| T | 125 (10.0%) | 13 (11.3%) | 16 (14.8%) | 7 (7.1%) | 18 (8.8%) |
| J | 127 (10.1%) | 9 (7.8%) | 11 (10.2%) | 11 (11.2%) | 21 (10.2%) |
| K | 133 (10.6%) | 7 (6.1%) | 12 (11.1%) | 10 (10.2%) | 12 (5.9%) |
| Other^{c} | 129 (10.3%) | 17 (14.8%) | 5 (4.6%) | 5 (5.1%) | 32 (15.6%) |
| Treatment Response | | | | | |
| Clinical Benefit | | | | | |
| CR | 168 (13.4%) | 14 (12.2%) | 21 (19.4%) | 5 (5.1%) | 36 (17.6%) |
| PR | 270 (21.5%) | 29 (25.2%) | 30 (27.8%) | 11 (11.2%) | 59 (28.8%) |
| dSD | 89 (7.1%) | 5 (4.3%) | 6 (5.6%) | 12 (12.2%) | 17 (8.3%) |
| No Clinical Benefit | | | | | |
| pSD | 117 (9.3%) | 9 (7.8%) | 6 (5.6%) | 22 (22.4%) | 30 (14.6%) |
| PoD | 610 (48.6%) | 58 (50.4%) | 45 (41.7%) | 48 (49.0%) | 63 (30.7%) |

IO-GEM Standard-of-Care Cohort

| NIVO Validation 2 | NIVO 1st-line only | IPI Validation | COMBO Validation |
|---|---|---|---|
| (N = 174) | (N = 153)^{a} | (N = 379) | (N = 196) |
| 117 (67.2%) | 103 (67.3%) | 240 (63.3%) | 121 (61.7%) |
| 57 (32.8%) | 50 (32.7%) | 139 (36.7%) | 75 (38.3%) |
| 62.2 (12.0) | 62.6 (12.1) | 60.6 (14.9) | 59.7(12.3) |
| 61.0 [20.0, 90.6] | 61.0 [20.0, 90.6] | 61.0 [19.9, 90.5] | 61.0 [19.0, 83.7] |
| 77 (44.3%) | 68 (44.4%) | 180 (47.5%) | 83 (42.3%) |
| 22 (12.6%) | 19 (12.4%) | 45 (11.9%) | 25 (12.8%) |
| 20 (11.5%) | 19 (12.4%) | 39 (10.3%) | 13 (6.6%) |
| 16 (9.2%) | 13 (8.5%) | 43 (11.3%) | 19 (9.7%) |
| 25 (14.4%) | 23 (15.0%) | 40 (10.6%) | 29 (14.8%) |
| 14 (8.0%) | 11 (7.2%) | 32 (8.4%) | 27 (13.8%) |
| 19 (10.9%) | 18 (11.8%) | 26 (6.9%) | 48 (24.5%) |
| 47 (27.0%) | 40 (26.1%) | 35 (9.2%) | 66 (33.7%) |
| 16 (9.2%) | 10 (6.5%) | 24 (6.3%) | 15 (7.7%) |
| 12 (6.9%) | 12 (7.8%) | 25 (6.6%) | 13 (6.6%) |
| 80 (46.0%) | 73 (47.7%) | 269 (71.0%) | 54 (27.6%) |

^{a}Unique patients (does not include n = 21 2nd-line patients treated with 1st-line IPI (n = 14) or COMBO (n = 7))
^{b}Value for participants missing age (n = 99) imputed using cohort median (61 years)
^{c}Other European haplogroups (I, X, W, R, V)

TABLE 6
Frequency of European Haplogroups in each analytic cohort compared
to the population frequency in the 1000 Genomes Project
Reference Population (1000 Genomes Project)

| Haplogroup | 1k Genomes (Reference) | % of Population |
|---|---|---|
| H | 201 | 49.8% |
| U | 79 | 19.6% |
| T | 36 | 8.9% |
| J | 33 | 8.2% |
| K | 24 | 5.9% |
| Other | 31 | 7.7% |
| Total | 404 | |

Total meta-analysis (BMS-067 and IO-GEM)

| Haplogroup | Overall Meta | % | p-value |
|---|---|---|---|
| H | 589 | 46.2% | 0.23 |
| U | 163 | 12.8% | 0.001 |
| T | 126 | 9.9% | 0.63 |
| J | 130 | 10.2% | 0.27 |
| K | 135 | 10.6% | 0.01 |
| Other | 132 | 10.4% | 0.14 |
| Total | 1275 | | |

p-value for two-sided t-test for difference in proportions

| | BMS-067 | | | BMS-067 | | | BMS-067 | | |
|---|---|---|---|---|---|---|---|---|---|
| Haplogroup | Nivo | % | p-value | Ipi | % | p-value | Combo | % | p-value |
| H | 102 | 45.7% | 0.38 | 49 | 50.0% | 0.99 | 98 | 47.8% | 0.71 |
| U | 31 | 13.9% | 0.09 | 16 | 16.3% | 0.56 | 24 | 11.7% | 0.02 |
| T | 29 | 13.0% | 0.14 | 7 | 7.1% | 0.72 | 18 | 8.8% | 0.99 |
| J | 20 | 9.0% | 0.85 | 11 | 11.2% | 0.45 | 21 | 10.2% | 0.48 |
| K | 19 | 8.5% | 0.29 | 10 | 10.2% | 0.20 | 12 | 5.9% | 0.99 |
| Other | 22 | 9.9% | 0.43 | 5 | 5.1% | 0.50 | 32 | 15.6% | 0.004 |
| Total | 223 | | | 98 | | | 205 | | |

IO-GEM (Validation)

| | IO-GEM | | | IO-GEM | | | IO-Gem | | |
|---|---|---|---|---|---|---|---|---|---|
| Haplogroup | Nivo | % | p-value | Ipi | % | p-value | Combo | % | p-value |
| H | 77 | 44.3% | 0.26 | 180 | 47.5% | 0.58 | 83 | 42.3% | 0.11 |
| U | 22 | 12.6% | 0.06 | 45 | 11.9% | 0.004 | 25 | 12.8% | 0.05 |
| T | 20 | 11.5% | 0.42 | 39 | 10.3% | 0.59 | 13 | 6.6% | 0.43 |
| J | 16 | 9.2% | 0.81 | 43 | 11.3% | 0.17 | 19 | 9.7% | 0.64 |
| K | 25 | 14.4% | 0.002 | 40 | 10.6% | 0.03 | 29 | 14.8% | 6.00E−04 |
| Other | 14 | 8.0% | 0.99 | 32 | 8.4% | 0.79 | 27 | 13.8% | 0.03 |
| Total | 174 | | | 379 | | | 196 | | |

Meta-analysis (BMS-067 and IO-GEM)

| | Nivo | | | Ipi | | | Combo | | |
|---|---|---|---|---|---|---|---|---|---|
| Haplogroup | Meta | % | p-value | Meta | % | p-value | Meta | % | p-value |
| H | 179 | 45.1% | 0.21 | 229 | 48.0% | 0.65 | 181 | 45.1% | 0.21 |
| U | 53 | 13.4% | 0.02 | 61 | 12.8% | 0.008 | 49 | 12.2% | 0.006 |
| T | 49 | 12.3% | 0.14 | 46 | 9.6% | 0.80 | 31 | 7.7% | 0.63 |
| J | 36 | 9.1% | 0.74 | 54 | 11.3% | 0.15 | 40 | 10.0% | 0.44 |
| K | 44 | 11.1% | 0.01 | 50 | 10.5% | 0.02 | 41 | 10.2% | 4.00E−02 |
| Other | 36 | 9.1% | 0.56 | 37 | 7.8% | 0.99 | 59 | 14.7% | 0.002 |
| Total | 397 | | | 477 | | | 401 | | |

TABLE 7
Patient demographics of the analytic cohort comparing NIVO and PEMBRO

| | | Anti-CTLA-4 | Anti-PD-1 | | Anti-CTLA-4/Anti-PD-1 |
|---|---|---|---|---|---|
| | Total | Ipilimumab | Nivolumab | Pembrolizumab | Ipilimumab/Nivolumab |
| | (N = 1,497) | (N = 477) | (N = 323) | (N = 296) | (N = 401) |
| Sex | | | | | |
| Male | 951 (63.5%) | 305 (63.9%) | 195 (60.4%) | 193 (65.2%) | 258 (64.3%) |
| Female | 546 (36.5%) | 172 (36.1%) | 128 (39.6%) | 103 (34.8%) | 143 (35.7%) |
| Age at treatment start^{a} (years) | | | | | |
| Mean (SD) | 60.9 (13.8) | 60.8 (14.9) | 60.0 (13.9) | 63.7 (12.5) | 59.6 (13.2) |
| Median | 62 | 61.7 | 62 | 62 | 62 |
| [Min, Max] | [19.0, 95.4] | [19.9, 90.5] | [20.0, 90.6] | [27.0, 95.4] | [19.0, 87.0] |
| European Haplogroup | | | | | |
| H | 697 (46.6%) | 229 (48.0%) | 145 (44.9%) | 142 (48.0%) | 181 (45.1%) |
| U | 192 (12.8%) | 61 (12.8%) | 44 (13.6%) | 38 (12.8%) | 49 (12.2%) |
| T | 145 (9.7%) | 46 (9.6%) | 44 (13.6%) | 24 (8.1%) | 31 (7.7%) |
| J | 159 (10.6%) | 54 (11.3%) | 29 (9.0%) | 36 (12.2%) | 40 (10.0%) |
| K | 149 (10.0%) | 50 (10.5%) | 32 (9.9%) | 26 (8.8%) | 41 (10.2%) |
| Other^{b} | 155 (10.4%) | 37 (7.8%) | 29 (9.0%) | 30 (10.1%) | 59 (14.7%) |
| Treatment Response | | | | | |
| Clinical Benefit | | | | | |
| CR | 221 (14.8%) | 31 (6.5%) | 52 (16.1%) | 54 (18.2%) | 84 (20.9%) |
| PR | 331 (22.1%) | 46 (9.6%) | 84 (26.0%) | 76 (25.7%) | 125 (31.2%) |
| dSD | 112 (7.5%) | 36 (7.5%) | 16 (5.0%) | 28 (9.5%) | 32 (8.0%) |
| No Clinical Benefit | | | | | |
| pSD | 117 (7.8%) | 47 (9.9%) | 21 (6.5%) | 6 (2.0%) | 43 (10.7%) |
| PD | 716 (47.8%) | 317 (66.5%) | 150 (46.4%) | 132 (44.6%) | 117 (29.2%) |

^{a}Value for participants missing age (n = 194) imputed using cohort median
^{b}Includes haplogroups I, R, V, W, and X

TABLE 8
Frequency of European Haplogroups in each analytic cohort
compared to the population frequency in the 1000 Genomes Project,
p-value for two-sided t-test for difference in proportions
Reference Population (1000 Genomes Project)

| | 1k Genomes | % of |
|---|---|---|
| Haplogroup | (Reference) | Population |
| H | 201 | 49.8% |
| U | 79 | 19.6% |
| T | 36 | 8.9% |
| J | 33 | 8.2% |
| K | 24 | 5.9% |
| Other | 31 | 7.7% |
| Total | 404 | |

Total analytic population (CheckMate-067 and IO-GEM)

| Haplogroup | Overall Meta | % | p-value |
|---|---|---|---|
| H | 697 | 46.6% | 0.25 |
| U | 192 | 12.8% | 0.0006 |
| T | 145 | 9.7% | 0.64 |
| J | 159 | 10.6% | 0.15 |
| K | 149 | 10.0% | 0.01 |
| Other | 155 | 10.4% | 0.11 |
| Total | 1497 | | |

CheckMate-067 and IO-GEM by ICI

| Haplogroup | IPI | % | p-value |
|---|---|---|---|
| H | 229 | 48.0% | 0.6 |
| U | 61 | 12.8% | 0.006 |
| T | 46 | 9.6% | 0.71 |
| J | 54 | 11.3% | 0.12 |
| K | 50 | 10.5% | 0.02 |
| Other | 37 | 7.8% | 0.96 |
| Total | 477 | | |
| Haplogroup | NIVO | % | p-value |
| H | 145 | 44.9% | 0.19 |
| U | 44 | 13.6% | 0.03 |
| T | 44 | 13.6% | 0.04 |
| J | 29 | 9.0% | 0.7 |
| K | 32 | 9.9% | 0.05 |
| Other | 29 | 9.0% | 0.05 |
| Total | 323 | | |
| Haplogroup | PEMBRO | % | p-value |
| H | 142 | 48.0% | 0.44 |
| U | 38 | 12.8% | 0.07 |
| T | 24 | 8.1% | 0.99 |
| J | 36 | 12.2% | 0.03 |
| K | 26 | 8.8% | 0.07 |
| Other | 30 | 10.1% | 0.12 |
| Total | 296 | | |
| Haplogroup | COMBO | % | p-value |
| H | 181 | 45.1% | 0.19 |
| U | 49 | 12.2% | 0.004 |
| T | 31 | 7.7% | 0.54 |
| J | 40 | 10.0% | 0.37 |
| K | 41 | 10.2% | 0.03 |
| Other | 59 | 14.7% | 0.002 |
| Total | 401 | | |

TABLE 9
Logistic regression results for treatment outcome (clinical benefit vs no
clinical benefit) for anti-CTLA-4 (IPI), anti-PD-1 (NIVO/PEMBRO),
and combined anti-CTLA-4/anti-PD-1 (COMBO) immune checkpoint
inhibitors (ICI) in the CheckMate-067 and IO-GEM cohorts (n = 1,497)

| Treatment Comparison | OR | 95% CI | p-value |
|---|---|---|---|
| Univariable | | | |
| IPI vs anti-PD-1^{a} | 3.23 | (2.49-4.22) | <2.00E−16 |
| IPI vs COMBO | 4.85 | (3.64-6.51) | <2.00E−16 |
| Anti-PD-1^{a} vs COMBO | 1.50 | (1.16-1.94) | 1.75E−03 |
| Multivariable^{b} | | | |
| IPI vs anti-PD-1^{a} | 3.27 | (2.51-4.28) | <2.00E−16 |
| IPI vs COMBO | 5.04 | (3.77-6.79) | <2.00E−16 |
| Anti-PD-1^{a} vs COMBO | 1.54 | (1.19-2.00) | 9.84E−04 |

^{a}Anti-PD-1 group includes both NIVO and PEMBRO-treated patients.
^{b}Multivariable regression model adjusted for age and sex.

TABLE 10
Logistic regression results for treatment outcome (clinical benefit vs
no clinical benefit) for anti-CTLA-4 (IPI), anti-PD-1 (NIVO and
PEMBRO separately), and combined anti-CTLA-4/anti-PD-1
(COMBO) immune checkpoint inhibitors (ICI) in the CheckMate-
067 and IO-GEM cohorts (n = 1,497)

| | OR | 95% CI | p-value |
|---|---|---|---|
| Treatment Comparison | | | |
| NIVO | | | |
| Univariable | | | |
| IPI vs Nivo | 2.86 | (2.12-3.89) | 1.14E−11 |
| Pembro vs Nivo | 0.78 | (0.57-1.06) | 0.12 |
| COMBO vs Nivo | 0.59 | (0.44-0.79) | 4.82E−04 |
| Multivariable^{a} | | | |
| IPI vs Nivo | 2.98 | (2.19-4.05) | 3.17E−12 |
| Pembro vs Nivo | 0.82 | (0.60-1.13) | 0.23 |
| COMBO vs Nivo | 0.59 | (0.44-0.80) | 5.65E−04 |
| Treatment Comparison | | | |
| PEMBRO | | | |
| Univariable | | | |
| IPI vs Pembro | 3.69 | (2.71-5.04) | <2.00E−16 |
| Nivo vs Pembro | 1.29 | (0.94-1.77) | 0.12 |
| COMBO vs Pembro | 0.76 | (0.56-1.03) | 0.08 |
| Multivariable^{a} | | | |
| IPI vs Pembro | 3.62 | (2.65-4.96) | 8.47E−16 |
| Nivo vs Pembro | 1.22 | (0.88-1.67) | 0.23 |
| COMBO vs Pembro | 0.72 | (0.53-0.98) | 0.04 |

^{a}Multivariable regression model adjusted for age and sex.

TABLE 11
Logistic regression results, stratified by mitochondrial haplogroup,
for treatment outcome (clinical benefit vs no clinical benefit) for
anti-CTLA-4 (IPI), anti-PD-1 (NIVO/PEMBRO), and combined
anti-CTLA-4/anti-PD-1 (COMBO) immune checkpoint inhibitors
(ICI) in the CheckMate-067 and IO-GEM cohorts (n = 1,497)

| | OR | 95% CI | p-value |
|---|---|---|---|
| Treatment Comparison | | | |
| Other MT-HGs | | | |
| Univariable | | | |
| IPI vs anti-PD-1^{a} | 3.64 | (2.76-4.83) | 1.71E−19 |
| IPI vs COMBO | 5.71 | (4.21-7.80) | 1.68E−28 |
| Anti-PD-1^{a} vs COMBO | 1.57 | (1.20-2.06) | 1.03E−03 |
| Multivariable^{b} | | | |
| IPI vs anti-PD-1^{a} | 3.69 | (2.79-4.91) | 1.37E−19 |
| IPI vs COMBO | 5.91 | (4.34-8.10) | 5.56E−29 |
| Anti-PD-1^{a} vs COMBO | 1.60 | (1.22-2.10) | 6.81E−04 |
| Treatment Comparison | | | |
| HG-T | | | |
| Univariable | | | |
| IPI vs anti-PD-1^{a} | 1.20 | (0.55-2.68) | 0.65 |
| IPI vs COMBO | 0.85 | (0.31-2.26) | 0.74 |
| Anti-PD-1^{a} vs COMBO | 0.70 | (0.27-1.73) | 0.45 |
| Multivariable^{b} | | | |
| IPI vs anti-PD-1^{a} | 1.19 | (0.54-2.66) | 0.67 |
| IPI vs COMBO | 0.92 | (0.33-2.47) | 0.86 |
| Anti-PD-1^{a} vs COMBO | 0.77 | (0.30-1.91) | 0.58 |

^{a}Anti-PD-1 group includes both NIVO and PEMBRO-treated patients.
^{b}Multivariable regression model adjusted for age and sex.

TABLE 12
Logistic regression results, stratified by haplogroup, for treatment
outcome (clinical benefit vs no clinical benefit) for anti-CTLA-4
(IPI), anti-PD-1 (NIVO and PEMBRO separately), and combined
anti-CTLA-4/anti-PD-1 (COMBO) immune checkpoint inhibitors
(ICI) in the CheckMate-067 and IO-GEM cohorts (n = 1,497)
2-5a. Other MT-HGs

| | OR | 95% CI | p-value |
|---|---|---|---|
| Treatment Comparison | | | |
| Other MT-HGs, NIVO | | | |
| Univariable | | | |
| IPI vs Nivo | 3.52 | (2.55-4.89) | 3.31E−14 |
| Pembro vs Nivo | 0.94 | (0.67-1.31) | 0.69 |
| COMBO vs Nivo | 0.62 | (0.45-0.84) | 2.65E−03 |
| Multivariable^{a} | | | |
| IPI vs Nivo | 3.68 | (2.66-5.13) | 7.86E−15 |
| Pembro vs Nivo | 0.99 | (0.71-1.40) | 0.98 |
| COMBO vs Nivo | 0.62 | (0.45-0.86) | 3.54E−03 |
| Treatment Comparison | | | |
| Other MT-HGs, PEMBRO | | | |
| Univariable | | | |
| IPI vs Pembro | 3.77 | (2.72-5.24) | 2.15E−15 |
| Nivo vs Pembro | 1.07 | (0.77-1.49) | 0.69 |
| COMBO vs Pembro | 0.66 | (0.48-0.91) | 0.01 |
| Multivariable^{a} | | | |
| IPI vs Pembro | 3.70 | (2.67-5.16) | 8.03E−15 |
| Nivo vs Pembro | 1.01 | (0.72-1.41) | 0.98 |
| COMBO vs Pembro | 0.63 | (0.45-0.86) | 4.41E−03 |

2-5b. HG-T

| | OR | 95% CI | p-value |
|---|---|---|---|
| Treatment Comparison | | | |
| HG-T, NIVO | | | |
| Univariable | | | |
| IPI vs Nivo | 0.61 | (0.23-1.54) | 0.30 |
| Pembro vs Nivo | 0.18 | (0.06-0.51) | 1.75E−03 |
| COMBO vs Nivo | 0.72 | (0.25-2.08) | 0.54 |
| Multivariable^{a} | | | |
| IPI vs Nivo | 0.61 | (0.23-1.55) | 0.30 |
| Pembro vs Nivo | 0.18 | (0.06-0.53) | 2.40E−03 |
| COMBO vs Nivo | 0.67 | (0.23-1.94) | 0.45 |
| Treatment Comparison | | | |
| HG-T, PEMBRO | | | |
| Univariable | | | |
| IPI vs Pembro | 3.44 | (1.25-9.99) | 0.02 |
| Nivo vs Pembro | 5.67 | (1.97-17.5) | 1.75E−03 |
| COMBO vs Pembro | 4.07 | (1.35-13.2) | 0.02 |
| Multivariable^{a} | | | |
| IPI vs Pembro | 3.31 | (1.20-9.67) | 0.02 |
| Nivo vs Pembro | 5.43 | (1.87-16.9) | 2.40E−03 |
| COMBO vs Pembro | 3.62 | (1.19-11.8) | 0.03 |

2-5c. Other MT-HGs vs HG-T NIVO or PEMBRO

| | OR | 95% CI | p-value |
|---|---|---|---|
| Treatment Comparison | | | |
| Other MT-HGs vs HG-T NIVO | | | |
| Univariable | | | |
| Other IPI vs HG-T Nivo | 1.00 | (0.45-2.03) | 0.99 |
| Other Nivo vs HG-T Nivo | 0.28 | (0.13-0.58) | 8.93E−04 |
| Other Pembro vs HG-T Nivo | 0.27 | (0.12-0.54) | 4.75E−04 |
| Other COMBO vs HG-T Nivo | 0.17 | (0.08-0.35) | 3.43E−06 |
| Multivariable^{a} | | | |
| Other IPI vs HG-T Nivo | 1.01 | (0.46-2.06) | 0.97 |
| Other Nivo vs HG-T Nivo | 0.28 | (0.12-0.56) | 7.15E−04 |
| Other Pembro vs HG-T Nivo | 0.27 | (0.12-0.56) | 6.87E−04 |
| Other COMBO vs HG-T Nivo | 0.17 | (0.08-0.35) | 3.03E−06 |
| Treatment Comparison | | | |
| Other MT-HGs vs HG-T | | | |
| PEMBRO | | | |
| Univariable | | | |
| Other IPI vs HG-T Pembro | 5.67 | (2.44-13.9) | 7.25E−05 |
| Other Nivo vs HG-T Pembro | 1.61 | (0.69-3.94) | 0.28 |
| Other Pembro vs HG-T Pembro | 1.50 | (0.65-3.69) | 0.35 |
| Other COMBO vs HG-T Pembro | 0.99 | (0.43-2.42) | 0.98 |
| Multivariableª | | | |
| Other IPI vs HG-T Pembro | 5.50 | (2.36-13.5) | 1.06E−04 |
| Other Nivo vs HG-T Pembro | 1.49 | (0.64-3.69) | 0.36 |
| Other Pembro vs HG-T Pembro | 1.49 | (0.64-3.67) | 0.37 |
| Other COMBO vs HG-T Pembro | 0.93 | (0.40-2.28) | 0.87 |

^{a}Multivariable regression model adjusted for age and sex.

TABLE 13
Clinical benefit for NIVO-based ICI treatment (NIVO or
COMBO) for patients on CheckMate-067,
stratified by MT-HG status (HG-T or other MT-HGs)

| | HG-T | | Other MT-HGs | |
|---|---|---|---|---|
| | CB | NCB | CB | NCB |
| 13a. Single-agent NIVO cohort | | | | |
| CheckMate-067 NIVO | | | | |
| PD-L1⁺ | 6 | 8 | 60 | 27 |
| PD-L1⁻ | 0 | 15 | 39 | 68 |
| 13b. Combined IPI/NIVO (COMBO) cohort | | | | |
| CheckMate-067 COMBO | | | | |
| PD-L1⁺ | 2 | 9 | 50 | 29 |
| PD-L1⁻ | 1 | 6 | 59 | 49 |
| 13c. NIVO and COMBO cohorts | | | | |
| CheckMate-067 NIVO and COMBO | | | | |
| PD-L1⁺ | 8 | 17 | 110 | 56 |
| PD-L1⁻ | 1 | 21 | 98 | 117 |

In the tables that follow, the Haplogroup Markers indicate the position in the mitochondrial genome and the corresponding base pair for top-level mitochondrial haplogroup definitions (those denoted by a single capital letter, e.g., U). These positions are based on mtDNA Tree build 17 (18 Feb. 2016 phylotree.org/tree/index.htm from which the entire description of haplogroups is incorporated herein by reference as it exists as of the effective filing date of this application). Unless in parentheses, each marker is present in at least 80% of the listed haplogroup. Parentheses indicate markers present in at least 50% but less than 80% of top level haplogroups. Lowercase nucleotide letters (a, c, t, g) indicate transversions (e.g., 186a). Lowercase “d” indicates a deletion following the (range of) position number(s) (e.g., 522d or 8281-8289d). Insertions are indicated by a dot after the mitochondrial reference sequence and the inserted base(s) (e.g., 2156.1A). Back mutations (markers that are reversions to the ancestral base) are indicated with an exclamation mark (!;!! for double back mutation) (e.g., A15301G!). Markers with the same value as the rCRS mitochondrial reference genome are indicated with an equals sign (e.g., 150C=). These markers are used to define the top level haplogroup, but will not be called as variants/SNPs by bioinformatics tools or genetic test results as they are identical to the reference sequence. The lack of any of the following high frequency variants, common to most haplogroup linages, may also be informative in determining haplogroup identity: 73G, 263G, 750G, 1438G, 2706G, 4769G, 7028T, 8860G, 11719A, 14766T, 15326G, & 16519C.

Haplogroup Markers Tables

| | Top Level | Ancestral Marker Motif | | Other Selected |
|---|---|---|---|---|
| Lineage | Haplogroup | (the “RSRS50”) | Haplogroup Markers | Markers |
| L | L0 | 247A, 750G, 769A, 825A, 1018A, 2758A, | 1048T, 3516a, 5442C, | (263A=) |
| | | 2885C, 3594T, 4104G, 4312T, 4769G, 7028T, | 6185C, 9042T, 9347G, | |
| | | 7146G, 7256T, 7521A, 8468T, 8655T, 8701G, | 9755A, 10589A, 12007A, | |
| | | 8860G, 9540C, 10398G, 10664T, 10688A, | 12720G | |
| | | 10810C, 10873C, 10915C, 11719A, 11914A, | | |
| | | 12705T, 13105G, 13276G, 13506T, 13650T, | | |
| | | 14766T, 15326G, 16187T, 16189C, 16223T, | | |
| | | 16230G, 16311C | | |
| | | (73G, 146C, 152C, 195C, 1438G, 2706G, | (522d, 4586C, 9818T, | |
| | | 16129A, 16519C) | 16172C) | |
| L | L1 | 73G, 152C, 247A, 263G, 750G, 769A, 825A, | 182T, 3666A, 7055G, | |
| | | 1018A, 2706G, 2758A, 2885C, 3594T, 4104G, | 7389C, 13789C, 14178C, | |
| | | 4769G, 7028T, 7146G, 7256T, 7521A, 8468T, | 14560A | |
| | | 8655T, 8701G, 8860G, 9540C, 10398G, | | |
| | | 10688A, 10810C, 10873C, 11719A, 12705T, | | |
| | | 13105G, 13506T, 13650T, 14766T, 15326G, | | |
| | | 16187T, 16189C, 16223T, 16278T, 16311C, | | |
| | | 16519C | | |
| | | (195C, 1438G, 16129A) | (151T, 186a, 189c, 316A, | |
| | | | 522d, 2395d, 5951G, | |
| | | | 6071C, 8027A, 9072G, | |
| | | | 10586A, 12810G, 13485G, | |
| | | | 14000A, 14911T, 16294T, | |
| | | | 16360T) | |
| L | L2 | 73G, 146C, 152C, 195C, 263G, 750G, 769A, | 2416C, 8206A, 9221G, | 150C=, 16311T= |
| | | 1018A, 1438G, 2706G, 3594T, 4104G, 4769G, | 10115C, 11944C, 13590A, | |
| | | 7028T, 7256T, 7521A, 8701G, 8860G, 9540C, | 15301A, 16390A | |
| | | 10398G, 10873C, 11719A, 12705T, 13650T, | | |
| | | 14766T, 15326G, 16223T, 16278T | | |
| | | (11914A) | (2789T, 7175C, 7274T, | |
| | | | 7771G, 12693G, 13803G, | |
| | | | 14566G, 15784C, 16294T, | |
| | | | 16309G) | |
| L | L3 | 73G, 263G, 750G, 1438G, 2706G, 4769G, | 15301A | 3594C=, 769G=, |
| | | 7028T, 8701G, 8860G, 9540C, 10398G, | | 1018G=, 16311T= |
| | | 10873C, 11719A, 12705T, 14766T, 15326G, | | |
| | | 16223T | | |
| | | (16519C) | (150T) | 3594C=, L4b2: |
| L | L4 | 73G, 263G, 750G, 769A, 1018A, 1438G, | 3918A, 15301A, 16362C | 146C |
| | | 2706G, 4769G, 7028T, 8701G, 8860G, 9540C, | | |
| | | 10398G, 10873C, 11719A, 12705T, 14766T, | | |
| | | 15326G, 16223T, 16311C | | |
| | | (146C, 16519C) | (244G, 315CC, 1413C, | |
| | | | 8104C, 9855G, 12609C, | |
| | | | 13470G, 16293T, 16355T, | |
| | | | 16399G) | |
| L | L5 | 73G, 247A, 263G, 750G, 769A, 825A, 1018A, | 182T, 3423C, 5147A, | Length variants in |
| | | 1438G, 2706G, 3594T, 4104G, 4769G, 7028T, | 7972G, 12432T, 12950G, | the 455-459 |
| | | 7256T, 7521A, 8655T, 8701G, 8860G, 9540C, | 14581C, 16148T, 16166G | region [459CCsor |
| | | 10398G, 10688A, 10810C, 10873C, 11719A, | | 455TTC or |
| | | 12705T, 13506T, 13650T, 14766T, 15326G, | | 455TTTC]. |
| | | 16129A, 16223T, 16278T, 16311C | | |
| | | (152C, 195C, 13105G, 16189C) | (522d, 709A, 851G, | |
| | | | 1822C, 5111T, 5656G, | |
| | | | 6182A, 6297C, 7424G, | |
| | | | 8155A, 8188G, 8582T, | |
| | | | 9305A, 9329A, 11025C, | |
| | | | 11881T, 12236A, 13722G, | |
| | | | 14212C, 14239T, 14905A, | |
| | | | 14971C, 15217A, 15884A, | |
| | | | 16355T, 16362C) | |
| L | L6 | 73G, 146C, 152C, 263G, 750G, 769A, 1018A, | 182T, 185C, 709A, 770T, | |
| | | 1438G, 2706G, 3594T, 4769G, 7028T, 7256T, | 961C, 1461G, 4964T, | |
| | | 8701G, 8860G, 9540C, 10398G, 10873C, | 5267C, 6002G, 6284G, | |
| | | 11719A, 12705T, 13650T, 14766T, 15326G, | 9332T, 10978G, 11116C, | |
| | | 16223T, 16278T, 16311C, 16519C | 11743T, 12771A, 13710G, | |
| | | | 14791T, 14959G, 15244G, | |
| | | | 15289C, 15301A, 15499T, | |
| | | | 16048A, 16224C | |
| | | | (265C, 309CCT, 310C) | |
| L3 \| M \| CZ | C | 73G, 263G, 750G, 1438G, 2706G, 4769G, | 249d, 489C, 3552A, | |
| | | 7028T, 8701G, 8860G, 9540C, 10398G, | 4715G, 7196a, 8584A, | |
| | | 10873C, 11719A, 11914A, 12705T, 14766T, | 9545G, 10400T, 13263G, | |
| | | 15326G, 16223T | 14318C, 14783C, 15043A, | |
| | | | 15301A, 15487T, 16298C, | |
| | | (16519C) | 16327T | |
| L3 \| M | D | 73G, 263G, 750G, 1438G, 2706G, 4769G, | 489C, 3010A, 4883T, | |
| | | 7028T, 8701G, 8860G, 9540C, 10398G, | 5178a, 8414T, 10400T, | |
| | | 10873C, 11719A, 12705T, 14766T, 15326G, | 14668T, 14783C, 15043A, | |
| | | 16223T | 15301A, 16362C | |
| L3 \| M | E | 73G, 263G, 750G, 1438G, 2706G, 4769G, | 489C, 3027C, 3705A, | |
| | | 7028T, 8701G, 8860G, 9540C, 10398G, | 4248C, 4491A, 7598A, | |
| | | 10873C, 11719A, 12705T, 15326G, 16223T, | 10400T, 10834T, 13254C, | |
| | | 16519C | 13626T, 14577C, 14783C, | |
| | | | 15043A, 15301A, 16362C, | |
| | | | 16390A | |
| | | | (6620C, 16291T) | |
| L3 \| M | G | 73G, 263G, 750G, 1438G, 2706G, 4769G, | 489C, 709A, 4833G, | |
| | | 7028T, 8701G, 8860G, 9540C, 10398G, | 5108C, 10400T, 14569A, | |
| | | 10873C, 11719A, 12705T, 14766T, 15326G, | 14783C, 15043A, 15301A, | |
| | | 16223T | 16362C | |
| | | | (5601T, 13563G) | |
| L3 | M | 73G, 263G, 750G, 1438G, 2706G, 4769G, | 489C, 10400T, 14783C, | |
| | | 7028T, 8701G, 8860G, 9540C, 10398G, | 15043A, 15301A | |
| | | 10873C, 11719A, 12705T, 14766T, 15326G, | | |
| | | 16223T | | |
| | | (16519C) | | |
| L3 \| M | Q | 73G, 263G, 750G, 1438G, 2706G, 4769G, | 89C, 489C, 4117C, 5460A, | |
| | | 7028T, 8701G, 8860G, 9540C, 10398G, | 5843G, 8790A, 8964T, | |
| | | 10873C, 11719A, 12705T, 14766T, 15326G, | 10400T, 12940A, 13500C, | |
| | | 16129A, 16311C | 14025C, 14783C, 15043A, | |
| | | | 15301A, 16144C, 16148T, | |
| | | | 16241G, 16265c | |
| | | (146C, 16223T) | (92A, 16343G) | |
| L3 \| M \| CZ | Z | 73G, 152C, 263G, 750G, 1438G, 2706G, | 249d, 489C, 4715G, | |
| | | 4769G, 7028T, 8701G, 8860G, 9540C, | 6752G, 7196a, 8584A, | |
| | | 10398G, 10873C, 11719A, 12705T, 14766T, | 9090C, 10400T, 14783C, | |
| | | 16223T | 15043A, 15301A, 15487T, | |
| | | | 15784C, 16185T, 16260T, | |
| | | | 16298C | |
| | | (15326G) | (310C) | |
| L3 \| N | A | 73G, 263G, 750G, 1438G, 2706G, 4769G, | 235G, 522d, 663G, 1736G, | |
| | | 7028T, 8860G, 11719A, 12705T, 14766T, | 4248C, 4824G, 8794T, | |
| | | 15326G, 16223T | 16290T, 16319A, 16362C | |
| | | (146C) | (153G, 310C, 8027A, | |
| | | | 12007A, 16111T) | |
| L3 \| N \| R | B | 73G, 263G, 750G, 1438G, 2706G, 4769G, | 16183c, 16217C | Branches B4 and |
| | | 7028T, 8860G, 11719A, 14766T, 15326G, | | B9: |
| | | 16189C, 16519C | | 10398A=, s8271 |
| | | | | 9bp deletions |
| | | | | [8271_8279del or |
| | | | | 8281_8289del]; |
| | | | | Branch B4: |
| | | | | 514_515delCA |
| | | (146C) | | Branch B5: 146T=, |
| | | | | 16217T= |
| L3 \| N \| R | F | 73G, 263G, 750G, 1438G, 2706G, 4769G, | 3970T, 6392C, 10310A, | |
| | | 7028T, 8860G, 11719A, 14766T, 15326G | 13928C, 16304C | |
| | | (16129A, 16519C) | (249d, 522d, 6962A, | |
| | | | 9053A, 10609C, 12406A, | |
| | | | 12882T, 13759A) | |
| L3 \| N \| R \| HV | H | 263G, 750G, 1438G, 4769G, 8860G, 15326G | | 2706A=, 7028C= |
| | | (16519C) | | |
| L3 \| N \| R | HV | 263G, 750G, 1438G, 2706G, 4769G, 7028T, | (310C) | 14766C= |
| | | 8860G, 15326G | | |
| L3 \| N | I | 73G, 263G, 750G, 1438G, 2706G, 4769G, | 199C, 204C, 250C, 1719A, | |
| | | 7028T, 8860G, 10398G, 11719A, 12705T, | 4529T, 8251A, 10034C, | |
| | | 14766T, 15326G, 16129A, 16223T, 16519C | 10238C, 12501A, 13780G, | |
| | | | 15043A, 15924G, 16391A | |
| L3 \| N \| R \| JT | J | 73G, 263G, 750G, 1438G, 2706G, 4769G, | 295T, 489C, 4216C, | |
| | | 7028T, 8860G, 10398G, 11719A, 14766T, | 11251G, 12612G, 13708A, | |
| | | 15326G | 15452a, 16069T, 16126C | |
| | | | (462T, 3010A, 14798C) | |
| L3 \| N \| R \| U | K | 73G, 263G, 750G, 1438G, 2706G, 4769G, | 1189C, 1811G, 3480G, | |
| | | 7028T, 8860G, 10398G, 11719A, 14766T, | 9055A, 9698C, 10550G, | |
| | | 15326G, 16311C, 16519C | 11299C, 11467G, 12308G, | |
| | | | 12372A, 14167T, 14798C, | |
| | | | 16224C | |
| | | | (315CC, 497T) | |
| L3 | N | 73G, 263G, 750G, 1438G, 2706G, 4769G, | | 8701A=, 9540T=, |
| | | 7028T, 8860G, 11719A, 12705T, 14766T, | | 10398A=, |
| | | 15326G, 16223T | | 10400C=, |
| | | | | 10873T=, |
| | | (16519C) | | 15301G= |
| L3 \| N | O | 73G, 263G, 750G, 1438G, 2706G, 4769G, | 6755A, 9140T, 16213A | |
| | | 7028T, 8860G, 11719A, 12705T, 14766T, | | |
| | | 15326G, 16223T | | |
| | | (152C, 16189C, 16519C) | (794C, 5563A, 16183c) | |
| L3 \| N \| R | P | 73G, 263G, 750G, 1438G, 2706G, 4769G, | 15607G | |
| | | 7028T, 8860G, 11719A, 14766T, 15326G | | |
| L3 \| N | R | 263G, 750G, 1438G, 2706G, 4769G, 7028T, | | 12705C=, |
| | | 8860G, 14766T, 15326G | | 16223C= |
| | | (73G, 11719A, 16519C) | | |
| L3 \| N | S | 73G, 263G, 750G, 1438G, 2706G, 4769G, | 8404C | |
| | | 7028T, 8860G, 11719A, 12705T, 14766T, | | |
| | | 15326G | | |
| | | (152C, 16223T) | | |
| L3 \| N \| R \| JT | T | 73G, 263G, 750G, 1438G, 2706G, 4769G, | 709A, 1888A, 4216C, | 10398A= |
| | | 7028T, 8860G, 11719A, 14766T, 15326G, | 4917G, 8697A, 10463C, | |
| | | 16519C | 11251G, 13368A, 14905A, | |
| | | | 15452a, 15607G, 15928A, | |
| | | | 16126C, 16294T | |
| | | | (11812G, 14233G) | |
| L3 \| N \| R | U | 73G, 263G, 750G, 1438G, 2706G, 4769G, | 11467G, 12308G, 12372A | |
| | | 7028T, 8860G, 11719A, 14766T, 15326G | | |
| L3 \| N \| R \| HV | V | 263G, 750G, 1438G, 2706G, 4769G, 7028T, | 72C, 4580A, 15904T, | |
| | | 8860G, 15326G | 16298C | |
| | | | (310C) | |
| L3 \| N | W | 73G, 195C, 263G, 750G, 1438G, 2706G, | 189G, 204C, 207A, 709A, | |
| | | 4769G, 7028T, 8860G, 11719A, 12705T, | 1243C, 3505G, 5046A, | |
| | | 14766T, 15326G, 16223T, 16519C | 5460A, 8251A, 8994A, | |
| | | | 11674T, 11947G, 12414C, | |
| | | | 15884C, 16292T | |
| | | | (194T) | |
| L3 \| N | X | 73G, 195C, 263G, 750G, 1438G, 2706G, | 153G, 1719A, 6221C, | |
| | | 4769G, 7028T, 8860G, 11719A, 12705T, | 6371T, 13966G, 14470C | |
| | | 14766T, 15326G, 16189C, 16223T, 16278T, | | |
| | | 16519C | | |
| | | | (225A) | |
| L3 \| N | Y | 73G, 263G, 750G, 1438G, 2706G, 4769G, | 5417A, 8392A, 14178C, | 16223C= |
| | | 7028T, 8860G, 10398G, 11719A, 12705T, | 14693G, 16126C, 16231C | |
| | | 14766T, 15326G | | |
| | | (146C, 16519C) | (310C, 3834A, 7933G, | |
| | | | 16266T) | |

Unique SNPs associated with sub-clades of top-level haplogroups J, T, U, or K (e.g., T2). These SNPs may be present in multiple sub-clades.

| | | | | | | | | | | | |
|---|---|---|---|---|---|---|---|---|---|---|---|
| 9966A | 7258C | 4025T | 16243C | 14325C | 11227T | 11497T | 14034C | 16129c | 3348G | 5567C | 8104C |
| 9932A | 7245G | 4013T | 16242T | 14323A | 11204C | 11539T | 14037G | 16134T | 334C | 5583T | 8119C |
| 9899C | 7211A | 4011T | 16242a | 14296G | 11177T | 11549T | 14053G | 16144C | 337G | 5592G | 8137T |
| 9872G | 7184G | 3992T | 16241G | 14281T | 11176A | 11566G | 14063C | 16154C | 3388a | 55a | 8164T |
| 9861C | 7150C | 3915A | 16240c | 14274G | 11087C | 11582G | 14070G | 16166d | 3397G | 560T | 8167C |
| 9843G | 709A | 3882A | 16239T | 14233G | 11084G | 11590G | 14094C | 16167T | 340T | 5686t | 8188G |
| 9836C | 7076G | 3867T | 16235G | 14182C | 11062T | 11620G | 14110C | 16168T | 3434G | 5746A | 8251A |
| 97A | 7022C | 3866C | 16231C | 14180C | 11016A | 11653G | 14139G | 16169a | 3480G | 5752d | 8260C |
| 9755A | 7001G | 385G | 16224C | 14133G | 11002G | 11674T | 14142g | 16173T | 3498T | 575T | 8276.1C |
| 9725C | 6998T | 384G | 16222T | 14118G | 10C | 11725G | 14167T | 16174T | 3504C | 5788C | 8277C |
| 9667G | 6962t | 3847C | 16218T | 1406C | 10966C | 11732C | 14178C | 16176T | 3507a | 5790a | 827G |
| 9635c | 6915A | 3834A | 16214T | 14061T | 10942G | 11734G | 14179A! | 16179T | 3507T | 5821A | 8282T |
| 9632G | 6911C | 3826C | 16213A | 14040A | 10822T | 11788T | 14179G | 16181G | 3513T | 5894c | 8296G |
| 9617G | 6893T | 3820T | 16195C | 14029G | 10750G | 11840T | 14198A | 16184T | 3535a | 5899.1C | 8308G |
| 9612A | 6887T | 3721G | 16193T | 14016A | 10685A | 11869a | 14212C | 16189a | 3546a | 5913A | 8395T |
| 9548A | 6830a | 3705A | 16192T | 13980A | 10601a | 11878C | 14215C | 16189C | 3552C | 5918C | 8407a |
| 9545G | 6716G | 3531A | 16189C! | 13973t | 10598G | 1187C | 14245T | 16189T!! | 3564G | 5984G | 8410T |
| 9494G | 6671C | 3456C | 16188T | 13966G | 10559G | 11881T | 14258A | 16192C! | 3591A | 5999C | 8413G |
| 9438A | 6635C | 3447G | 16187T! | 13965C | 10499G | 11893G | 14259A | 16206c | 3654T | 6023A | 8428a |
| 9422c | 6620C | 3398C | 16186T | 13956G | 10497T | 1189C | 14278C | 16207G | 3666A | 6041T | 8433C |
| 9324G | 6554T | 3394C | 16172C | 13953C | 10496G | 11923G | 14305A | 16209C | 3672G | 6045T | 8440G |
| 930A | 6524C | 3350C | 16169T | 13934T | 10490C | 11929C | 14308C | 16214a | 3688c | 6047A! | 8473C |
| 9300A | 6491T | 3338C | 16163G | 13933G | 10463C | 11935C | 14319C | 16219A! | 368G | 6047G | 8490C |
| 9254G | 6489a | 3308g | 16153A | 13928c | 10454C | 11938T | 14384A | 16219G | 3696T | 6053T | 8496C |
| 9234G | 6464a | 3290C | 16148T | 13917G | 10446G | 1193C | 14388G | 16223T! | 3702G | 6060G | 8522T |
| 9233C | 6445T | 321C | 16147T | 13899C | 10410C | 11941G | 14420T | 16230G! | 3714G | 6071C | 8531G |
| 9224C | 6425C | 3213G | 16146G | 13879C | 10410a | 11947G | 1442A | 16234T | 3720G | 6146G | 8557c |
| 9219g | 6392C | 3203G | 16145A | 13834G | 10398G! | 11965T | 14518G | 16245T | 3738T | 6164T | 8567C |
| 91T | 6383A | 319C | 16140C | 13830C | 10398A!! | 11971T | 14530C | 16246t | 373G | 6182A | 8574T |
| 9192A | 634C | 310.1T | 16136C | 13813A | 10389C | 11984C | 14533T | 16259T | 3741T | 6221C | 8598C |
| 9181G | 6345C | 3010A | 16129A! | 13791T | 10376G | 12017G | 14562T | 16270C! | 3750T | 6257A | 8610C |
| 9180G | 6293C | 2969G | 16126T! | 13785T | 10345C | 12019T | 14574T | 16278C!! | 3768G | 6260G! | 8642G |
| 9145A | 6261A | 295T | 16126C | 13768C | 10334T | 12022T | 14577C | 16293G | 3777C | 6284G | 8676T |
| 9139A | 6260A | 2850C | 16114T | 13759A | 10321C | 12094T | 14577g | 16295T | 3816G | 629C | 8684T |
| 9120G | 6249A | 279C | 16104T | 13753C | 10289G | 12097T | 14605t | 16311C | 3849A | 6341T | 8701G! |
| 9117C | 6216C | 271T | 16092C | 13743C | 10274C | 12103a | 14620T | 16311T!! | 3861G | 6359G | 8702T |
| 9111C | 6152C | 2707c | 16086C | 13722G | 10237C | 12106T | 14684T | 16318t | 394T | 6367C | 8705C |
| 9103C | 6045a | 2706A | 16069T | 13708A | 10227C | 1211A | 146T!! | 16320T | 3969T | 6386T | 8706G |
| 9090C | 6026A | 266C | 16066G | 13692T | 10192T | 12134C | 1472A | 16327T | 4021G | 63C | 8766T |
| 9083C | 59C | 247A! | 16063C | 13681G | 10172A | 12135a | 14767C | 16336A | 4053G | 6407C | 8778T |
| 9055A | 5964C | 242T | 16051G | 13590A | 10166C | 12136C | 14831A | 16342C | 4059T | 6413C | 8790A |
| 9052G | 593C | 2404C | 159C | 13581C | 10143A | 12161C | 14866T | 16343G | 4062C | 6452T | 8812G |
| 9041G | 5915T | 2387C | 15965G | 13557G | 10101C | 12188C | 14869c | 16352C | 408a | 6455T | 8818T |
| 9016G | 5836G | 2380T | 15947G | 13506T! | 10084C | 12246T | 14883T | 16353T | 4093G | 6461G | 8842c |
| 8994A | 5817T | 235G | 15941C | 13500C | 1007A | 12297C | 14893G | 16354T | 4113A | 64T | 8856c |
| 8974T | 5811G | 2308G | 15936t | 13470G | 1005C | 12308A! | 14921A | 16356C | 4129G | 6518T | 8870C |
| 8958T | 57C | 228A | 15935G | 13434G | 10029G | 12331G | 14926G | 16356T! | 4132A | 6527G | 8901G |
| 8940T | 5774C | 222T | 15928A | 13419G | 10031C | 12338C | 14927G | 16362T! | 4137T | 6528T | 8933T |
| 8934T | 5773A | 215G | 15924G | 13398G | 10127G | 12346T | 14935C | 16398A | 4158G | 6546T | 8938G |
| 8923G | 5747G | 2158C | 15916C | 13392C | 10142T | 12372A | 14956C | 16399G | 4172a | 6584T | 8950A |
| 8868C | 573.XC | 214G | 15913T | 13368A | 10154G | 12376g | 14971C | 16400T | 4188G | 6599G | 8952C |
| 8865A | 5656G | 2141C | 15894A | 13308G | 10202T | 12491T | 15031T | 16428A | 4209C | 6629G | 896G |
| 8864C | 5633T | 2140A | 15884A | 13281C | 10203A | 12501A | 15049T | 16463G | 4216C | 663G | 8982G |
| 8854A | 5585A | 207A | 15853T | 13260C | 10253C | 12530G | 15061G | 16465T | 4224T | 6665T | 9006G |
| 8853G | 5582G | 204C | 15836G | 13188T | 10262G | 12535T | 15077A | 16497G | 4295G | 6674C | 9031T |
| 8847T | 5580C | 203c | 15812A | 13145A | 10283G | 12582G | 15097C | 16524G | 431a | 6680C | 9070g |
| 8843C | 5558G | 200G | 15787C | 13135A | 10301G | 12603T | 15106A | 16525G | 4336C | 6719C | 9093G |
| 8839A | 5527G | 199C | 15784C | 13132T | 10326a | 12609C | 15115C | 1692t | 437T | 6836T | 9094T |
| 8838A | 5501G | 198T | 15758G | 13032G | 10336C | 12615G | 15172A | 1694C | 4502C | 6845T | 9100G |
| 8775T | 55.1T | 1977C | 15725T | 13020C | 10364A | 12616C | 15191C | 16t | 455.1T | 6881G | 9101C |
| 8734a | 5498G | 195C! | 15679G | 13015C | 10397G | 12618A | 1520C | 1700C | 4553C | 6890G | 9128C |
| 8730G | 5493C | 194T | 15672C | 12957C | 103A | 12630A | 15217A | 1719A | 455d | 6899A | 9148C |
| 8727T | 5483C | 189G | 15670C | 12891T | 1040C | 12654G | 15218G | 1721T | 4561C | 6905G | 9196A |
| 8715C | 5471A | 188G | 15662G | 12879C | 10478T | 12678C | 15244G | 1809C | 4562G | 6920a | 9266A |
| 8711G | 5463T | 1888A | 15617A | 12771A | 1047G | 12681C | 15289C | 1811A! | 4592C | 6929G | 9287A |
| 8697A | 5460A | 1871G | 15607G | 12753G | 10506G | 12696C | 15301A!! | 1819C | 4616T | 6938T | 9293T |
| 8659G | 5442C | 185A | 15607A! | 12741T | 10544T | 12720G | 15314A | 1822C | 4640a | 6956C | 9299G |
| 8633G | 5426C | 1850C | 15601C | 12672G | 10550G | 12727C | 15325G | 1834C | 4640T | 6975C | 9335a |
| 8596G | 5414G | 1836G | 15596A | 12634G | 10589A | 12738g | 15326A | 183G | 4646C | 7028C | 9338G |
| 8572A | 5378G | 1829G | 15530C | 12633a | 10619T | 12768G | 15327T | 1842G | 4654T | 7031T | 9365T |
| 8558T | 5322c | 1811G | 15514C | 12612G | 10654T | 12793C | 15355A | 1926t | 4655A | 7055G | 9371T |
| 8557A | 5297T | 1766C | 15511C | 12570G | 10676T | 12795A | 15373G | 195T!! | 466C | 7082T | 9377G |
| 8530G | 5277C | 1733Ta | 15509G | 12561A | 10692T | 12842C | 15380G | 1978G | 4674G | 7085C | 9389G |
| 8504C | 5276G | 1709A | 15499T | 12557T | 10697T | 12843C | 15381T | 2083C | 4703C | 7109T | 93G |
| 8460G | 5262A | 16527T | 15490T | 12477C | 10698T | 12870T | 15383C | 217C | 471C | 7118G | 9428C |
| 8455T | 5237A | 16526A | 15466A | 12453C | 10700G | 12937G | 15431A | 2217T | 4732G | 7151T | 942G |
| 8419C | 5231A | 16438A | 15454C | 12441C | 10709c | 12950c | 15497A | 2218T | 4736C | 7202G | 9448G |
| 8412C | 5201C | 16390A | 15453C | 12408C | 10733T | 12954C | 15519C | 2225a | 4745t | 7226A | 9469T |
| 8404C | 5198G | 16370A | 15452a | 12406A | 10754G | 13002a | 15533G | 2246G | 4748T | 723c | 9477A |
| 8386T | 5187T | 16368C | 15445C | 12397G | 10768G | 13017G | 15544a | 2294G | 4775G | 723G | 9480C |
| 8290A | 5162C | 16366T | 15442G | 12396C | 10784G | 1303A | 15544T | 234G | 4796T | 7253G | 94A |
| 8281-8289d | 5147A | 16362C | 153G | 12379T | 10819G | 13104G | 15553A | 2352C | 4811G | 7265G | 9571T |
| 8271G | 513A | 16357C | 15367T | 12373G | 10858C | 13105G! | 15613G | 2405.1C | 4820A | 7277G | 9575A |
| 8270T | 512G | 16355T | 1530G | 12363T | 10861C | 131110 | 15625a | 2483C | 4843T | 7278C | 960.1C |
| 8269A | 5108C | 16335G | 152T!! | 12358G | 10876G | 13117G | 15626T | 249d | 4856C | 7340A | 960.XC |
| 8256C | 507C | 16325C | 152C!!! | 12311C | 10885C | 13158G | 15631G | 249G | 4924c | 7382A | 960d |
| 8230T | 5054A | 16324C | 152C! | 12308G | 108G | 13161C | 15632T | 2526T | 4936T | 7385G | 9614G |
| 8222C | 5024T | 16319A | 15262C | 12223G | 10907C | 13194A | 15634G | 252C | 4965G | 7388G | 961C |
| 8155A | 499A | 16311C! | 15257A | 12172G | 10927C | 13269G | 15650A | 262T | 4970G | 7403G | 9647C |
| 8152A | 4991A | 16309G | 15213C | 12171G | 10972G | 13285G | 15652G | 263A | 497T | 742C | 965.XC |
| 8149G | 4959A | 16304T! | 151T | 12127A | 10978G | 13293T | 15661T | 2672G | 498d | 745G | 9656C |
| 8078A | 4931T | 16304C | 15199T | 12123T | 11009C | 13326C | 15693C | 2707G | 5081C | 7471T | 9664G |
| 8041G | 4924A | 16301T | 15172t | 12083g | 11013T | 13351T | 15721C | 270G | 508G | 750A | 9670G |
| 8020A | 4917G | 16300G | 15148A | 12028C | 11017C | 13359A | 15734A | 2755G | 5093C | 7559G | 9682C |
| 7984A | 4917A! | 16298C | 15119A | 12020T | 11025C | 13395G | 15777A | 2757G | 5120G | 7569G | 9698C |
| 7963G | 489C | 16297C | 15113G | 12007A | 11038G | 13401C | 15781T | 2766a | 512c | 7581C | 9716C |
| 789C | 4853A | 16296T | 15110A | 11944C | 11050C | 13404C | 15789T | 2772T | 5130C | 7624C | 9738A |
| 7897A | 482C | 16294T | 150T | 11914A! | 11071T | 13413G | 15790T | 2792G | 5153G | 7642A | 9767T |
| 7891T | 4829G | 16294C! | 15090C | 11857T | 11107T | 1341T | 15791G | 280g | 5165T | 7657C | 9770C |
| 7873T | 4823C | 16292T | 15067C | 11827C | 11116C | 13420T | 15799G | 282C | 516T | 7700T | 9777A |
| 7870C | 4808T | 16291T | 15043A | 11818G | 11143T | 13422G | 15803A | 2831A | 5177A | 7705C | 9779T |
| 7853A | 4802C | 16290T | 1503A | 11812G | 11151T | 13431T | 15850g | 2836T | 517t | 7729G | 9800C |
| 7789A | 4790G | 16288C | 15028a | 11812A! | 11167G | 13440T | 15862C | 285T | 5186t | 7735G | 9801A |
| 7783C | 4772C | 16287T | 15014C | 11800G | 11172G | 13528G | 15880G | 291.1A | 5240G | 7768G | 980C |
| 7747T | 4739T | 16286T | 14950T | 11797G | 11191T | 13565T | 15883A | 295a | 5250C | 7775A | 9852G |
| 7711C | 469T | 16284G | 14905A | 11778A | 11197T | 13569C | 15891T | 296T | 5261A | 7792T | 9852t |
| 769A! | 4696C | 16278T! | 14861A | 11647T | 11233C | 13602C | 15892C | 3027C | 5263T | 7805A | 988A |
| 7679C | 4695C | 16274A | 14839G | 11623T | 11268T | 13617C | 15900C | 309d | 5264T | 7843G | 9891C |
| 7521A! | 4688C | 16271C | 14836G | 11533T | 11272G | 13630G | 15903G | 3105G | 5265G | 7859A | 9896G |
| 751t | 462T | 16270T | 14798C | 11518A | 11296T | 13635C | 15905C | 310C | 5298G | 7894G | 9903C |
| 7501C | 458T | 16269c | 14793G | 11503T | 11299C | 13637G | 15907G | 3116T | 5319G | 7912A | 9938C |
| 7476T | 456T | 16266T | 14769G | 114T | 11326T | 13656C | 15927A | 3158.1T | 5319t | 7918T | 9948A |
| 73A | 4491A | 16265G | 146C! | 11467G | 11329G | 13674C | 15930A | 3170a | 533G | 7927g | 9962A |
| 7391C | 4464A | 16265c | 14687G | 11440A | 11332T | 13710G | 15944d | 3192T | 5360T | 7930G | 9977C |
| 7364G | 4454C | 16263C | 14605G | 11416T | 11339C | 13734C | 15946T | 3197C | 5390G | 793T | 9989C |
| 7337A | 430C | 16261T | 14569A | 11386C | 11348T | 13740C | 15954c | 3198G | 5437T | 794a | |
| 7310C | 4232C | 16261a | 14544A | 11383C | 11350G | 13789C | 16048A | 3204T | 5452T | 7960c | |
| 7302C | 4225G | 16260T | 14500G | 11377A | 11353C | 13802T | 16067T | 3212T | 5465C | 7978T | |
| 7299G | 41T | 16257T | 14470C | 11287C | 11374G | 13827G | 16074G | 3229.1A | 5486T | 8005C | |
| 7295G | 41C! | 16257d | 14455T | 11266T | 11465C | 13928A | 16079T | 324T | 5495C | 8023C | |
| 7283C | 4155T | 16256T | 143A | 11252G | 11470G | 13967T | 16093C | 325T | 5508C | 8027A | |
| 7269A | 4107T | 16249C | 1438A | 11251G | 11485C | 14002G | 16111T | 3316A | 5554a | 8065A | |
| 7268C | 4084A | 16248T | 14364A | 11242g | 11491G | 14003T | 16114a | 3337A | 5557C | 8098G | |

1. A method comprising determining whether or not an individual is likely to respond to treatment with one or more immune checkpoint inhibitors (ICI's) based on a determination of a mitochondrial haplogroup for the individual, and wherein the haplogroup of the individual is determined prior to receiving immune checkpoint inhibition.

2. The method of claim 1, wherein the haplogroup that is determined is a T haplogroup, a K haplogroup, a U haplogroup, or a J haplogroup.

3. The method of claim 2, wherein the wherein the haplogroup that is determined is the T haplogroup or the K haplogroup or the U haplogroup, and wherein the T haplogroup or the K haplogroup or the U haplogroup indicates the individual is likely to be resistant to a particular anti-PD-1 ICI antibody as a monotherapy.

4. The method of claim 3, further comprising administering to the individual an ICI that is not the particular ICI anti-PD-1 antibody as a monotherapy.

5. The method of claim 2, wherein the haplogroup is the J haplogroup, and wherein the J haplogroup indicates the individual will likely be sensitive to an anti-PD1 ICI.

6. The method of claim 5, further comprising administering the anti-PD-1 ICI to the individual.

7. The method of claim 2, wherein the wherein the haplogroup that is determined is the T haplogroup or the K haplogroup or the U haplogroup, and wherein the T haplogroup or the K haplogroup or the U haplogroup indicates the individual is likely to be sensitive to an anti-PD-1 ICI antibody that is not nivolumab, and wherein optionally the anti-PD-1 antibody that is not the nivolumab is used as a monotherapy.

8. The method of claim 7, further comprising administering the anti-PD-1 ICI that is not the nivolumab to the individual.

9. The method of claim 1, wherein determining the haplogroup comprises determining one or more of the markers as defined by the haplogroup tables herein.

10. The method of claim 1, comprising quantitative next-generation sequencing analysis of RNA expressed from a locus that includes MTRNR2L8, or ATAC-seq chromatin accessibility of the locus that includes the MTRNR2L8, to thereby indicate the individual has the T haplogroup.

11. The method of claim 10, further comprising administering to the individual an anti-PD-1 ICI that the individual is likely to be sensitive to based on a determined haplogroup, and wherein optionally the anti-PD-1 ICI that the individual is likely to be sensitive is not nivolumab used as a monotherapy.
